(19) United States
(12) Patent Application Publication
(10) Pub. No.: US 2025/0144213 A1
(43) Pub. Date: May 8, 2025

(54) CIRCULAR RNA AND USE THEREOF

(71) Applicant: UTC Therapeutics (Shanghai) Co., Ltd., Shanghai (CN)

(72) Inventors: Yangbing Zhao, Shanghai (CN); Gengzhen Zhu, Shanghai (CN); Changshun Wu, Shanghai (CN)

(73) Assignee: UTC Therapeutics (Shanghai) Co., Ltd., Shanghai (CN)

(21) Appl. No.: 18/730,234

(22) PCT Filed: Jan. 19, 2023
(86) PCT No.: PCT/CN2023/073206

§ 371 Date: Jul. 18, 2024

(30) Foreign Application Priority Data
Jan. 19, 2022 (WO) PCT/CN2022/072823

(51) Int. Cl. A61K 40/41; A61K 40/11; A61K 40/31; C07K 14/705; C07K 14/725; C07K 16/28; C12N 15/85

(52) U.S. Cl. CPC A61K 40/41; A61K 40/11; A61K 40/31; C07K 14/7051; C07K 14/7055; C07K 16/2878; C12N 15/85; C07K 2317/24; C07K 2317/622; C07K 2319/03; C12N 2800/107; C12N 2830/50; C12N 2840/203

(57) ABSTRACT

Provided relates to a circular RNA comprising an internal ribosome entry site (IRES) element, a protein coding sequence and a poly A. Provided also relates to the precursor RNA, the vector and the method for producing the circular, and the use of the circular RNA.

### CROSS REFERENCE TO RELATED APPLICATIONS

This is the U.S. National Stage of International Application No. PCT/CN2023/073206, filed Jan. 19, 2023, which was published in English under PCT Article 21(2), and claims priority to PCT patent application PCT/CN2022/072823, filed on Jan. 19, 2022 and entitled “Circular RNA and Use Thereof.” Both of the aforementioned PCT applications are incorporated herein by reference in their entireties.

### INCORPORATION OF SEQUENCE LISTING

The Sequence Listing is submitted as an XML file named “Sequence Listing_112298-01.xml,” created on Jul. 18, 2024, (˜1,500,924 bytes), which is incorporated herein by reference.

### FIELD

The present invention relates to the field of molecular biology and biological engineering technology, in particular, to a circular RNA.

### BACKGROUND

Messenger RNA (mRNA) has broad potential for application in biological systems. However, one fundamental limitation to its use is its relatively short half-life in biological systems. Recently, circular RNA (circRNA) is found to be superior in the duration of protein expression than conventional linear mRNA. CircRNAs lack the free ends necessary for exonuclease-mediated degradation, rendering them resistant to several mechanisms of RNA turnover and granting them extended lifespans as compared to their linear mRNA counterparts. Recently, the permuted group 1 catalytic intron-based system has been used to circularize a wide range of RNA sequences in vitro, with circularization efficiencies reported to reach nearly 100%.

### SUMMARY

One aspect of the present invention provides a circular RNA comprising, in the following order, an internal ribosome entry site (IRES) element, a protein coding sequence and a poly A.

In some embodiments, the protein is for therapeutic use.

In some embodiments, the protein is an antigen, an antibody, a chimeric antigen receptor (CAR) or a T cell receptor (TCR); preferably, the antibody is a scFv.

In some embodiments, the binding domain of the CAR is an anti-mesothelin scFv.

In some embodiments, the protein comprises an antibody or a CAR comprising the antibody as a binding domain, wherein the antibody specifically binds to mesothelin, CD123, BCMA, HER2, IL13Ra2, B7H3 or CD40, such as those antibodies and CARs provided and described in the present invention.

In some embodiments, the protein comprises a LACOSTIM as described in the present invention, or comprises a first protein and a second protein, wherein the first protein comprises an antibody, a chimeric antigen receptor (CAR) or a T cell receptor (TCR) as described in the present invention and the second protein comprises a LACOSTIM as described in the present invention.

In some embodiments, the LACOSTIM comprises a first domain that activates an antigen-presenting cell (APC) and a second domain that activates an immune effector cell, wherein (i) the first domain comprises (a) a ligand that binds to an activation receptor of the APC, or a receptor-binding fragment thereof, or (b) an antibody that binds to an activation receptor of the APC, or an antigen-binding fragment thereof; and (ii) the second domain comprises (a) a co-stimulatory receptor of the immune effector cell, or a functional fragment thereof, (b) a co-stimulatory ligand of the immune effector cell, or a receptor-binding fragment thereof, or (c) an antibody that binds to a co-stimulatory receptor of the immune effector cell, or an antigen-binding fragment thereof.

In some embodiments, the first domain is linked to the N-terminus or C-terminus of the second domain. In some embodiments, the first domain and the second domain are linked via a linker.

In some embodiments, the polyA is at least 45 nucleotides in length.

In some embodiments, the polyA is at least 70 nucleotides in length.

Another aspect of the present invention provides a precursor RNA for producing any one of above circular RNA, any one of above precursor RNA comprising a circularizing element, an internal ribosome entry site (IRES) element, a protein coding sequence and a poly A.

In some embodiments, the circularizing element comprises a first intron sequence on the 5′ of the internal ribosome entry site (IRES) element and a second intron sequence on the 3′ of the poly A.

In some embodiments, the first intron sequence and the second intron sequence are derived from Group I or Group II intron self-splicing sequences.

In some embodiments, the first intron element comprises a 3′ Group I intron fragment containing a 3′ splice site dinucleotide, and the second intron element comprises a 5′ Group I intron fragment containing a 5′ splice site dinucleotide.

In some embodiments, the precursor RNA further comprises a 5′ spacer sequence between the first intron element and the internal ribosome entry site (IRES) element, and a 3′ spacer sequence between the polyA and the second intron element.

In some embodiments, the precursor RNA further comprises a 5′ homology arm external to the first intron element and a 3′ homology arm external to the second intron element.

Another aspect of the present invention provides a vector for producing any one of above precursor RNA, wherein the vector comprises a DNA template for the precursor RNA.

In some embodiments, the vector further comprises an RNA polymerase promoter.

Another aspect of the present invention provides a method of producing a circular RNA, the method comprising circularizing any one of above precursor RNA to produce the circular RNA.

In some embodiments, the method comprises transcribing a vector comprising DNA template for any one of above precursor RNA to obtain the precursor RNA before the circularization.

In some embodiments, the transcription step is performed in a cell or in a cell-free system.

In some embodiments, the method further comprises purifying the circular RNA.

In some embodiments, the circular RNA is purified through oligo dT-based capturing.

Another aspect of the present invention provides a cell or a cell population comprising any one of above circular RNA.

In some embodiments, the cell or a cell population comprising a first circular RNA and a second circular RNA, wherein the protein coding sequence of the first circular RNA encodes an antibody, a chimeric antigen receptor (CAR) or a T cell receptor (TCR) as described in the present invention and the protein coding sequence of the second circular RNA encodes a LACOSTIM as described in the present invention. Another aspect of the present invention provides a method for expressing a protein in a cell, the method comprising introducing any one of above circular RNA, any one of above precursor RNA or any one of above vector into a host cell and expressing the protein encoded by the protein coding sequence in the circular RNA.

Another aspect of the present invention provides a method of producing a protein, the method comprising:
(a) translating any one of above circular RNA to produce the protein encoded by the protein coding sequence of the circular RNA; and
(b) purifying the protein.

In some embodiments, the translation step is performed in a cell or in a cell-free system.

In some embodiments, the step (a) comprises introducing any one of above circular RNA, any one of above precursor RNA or any one of above vector into a host cell and translating the circular RNA in the host cell to produce the protein.

Another aspect of the present invention provides a pharmaceutically composition comprising:
(a) any one of above circular RNA, any one of above precursor RNA, any one of above vector, or any one of above cell or cell population; and
(b) a pharmaceutically acceptable carrier.

Another aspect of the present invention provides a composition comprising:
(a) any one of above circular RNA, any one of above precursor RNA, any one of above vector; and
(b) a delivery carrier.

Another aspect of the present invention provides a method of purifying any one of above circular RNA, the method comprising purifying the circular RNA through oligo dT-based capturing.

Another aspect of the present invention provides a method of treating a disease in a subject in need thereof, comprising administering to the subject a therapeutically effective amount of any one of above circular RNA, any one of above precursor RNA, any one of above vector or any one of above cell or cell population.

In some embodiments, the disease is a tumor, a cancer, a virus infection or an autoimmune disease.

In some embodiments, the cancer expresses mesothelin, CD123, BCMA, HER2, IL13Ra2 or B7H3.

In some embodiments, the cancer is a solid tumor or a hematological cancer.

In some embodiments, the cancer is acute myeloid leukemia (AML), B-acute lymphoid leukemia (B-ALL), T-acute lymphoid leukemia (T-ALL), B cell precursor acute lymphoblastic leukemia (BCP-ALL) or blastic plasmacytoid dendritic cell neoplasm (BPDCN), non-Hodgkin's lymphoma, chronic lymphocytic leukemia, acute lymphocytic leukemia, human B-cell precursor leukemia, multiple myeloma or malignant lymphoma.

In some embodiments, the cancer is mesothelioma, pancreatic cancer, ovarian cancer, lung cancer, breast cancer, stomach cancer, cervical cancer, uroepithelial cancer, esophageal cancer, bladder cancer, colorectal cancer, endometrial cancer, kidney cancer, head and neck cancer, sarcoma, glioblastoma, prostate cancer, thyroid cancer or glioma.

Another aspect of the present invention provides use of any one of above circular RNA, any one of above precursor RNA, any one of above vector or any one of above cell or cell population in preparation of a medicament for treating a disease in a subject in need thereof.

In some embodiments, the disease is a tumor, a cancer, a virus infection or an autoimmune disease.

In some embodiments, the cancer expresses mesothelin, CD123, BCMA, HER2, IL13Ra2 or B7H3.

In some embodiments, the cancer is a solid tumor or a hematological cancer.

In some embodiments, the cancer is acute myeloid leukemia (AML), B-acute lymphoid leukemia (B-ALL), T-acute lymphoid leukemia (T-ALL), B cell precursor acute lymphoblastic leukemia (BCP-ALL) or blastic plasmacytoid dendritic cell neoplasm (BPDCN), non-Hodgkin's lymphoma, chronic lymphocytic leukemia, acute lymphocytic leukemia, human B-cell precursor leukemia, multiple myeloma or malignant lymphoma.

In some embodiments, the cancer is mesothelioma, pancreatic cancer, ovarian cancer, lung cancer, breast cancer, stomach cancer, cervical cancer, uroepithelial cancer, esophageal cancer, bladder cancer, colorectal cancer, endometrial cancer, kidney cancer, head and neck cancer, sarcoma, glioblastoma, prostate cancer, thyroid cancer or glioma.

In the present invention, we introduce poly A sequences into the circRNA molecule. The circRNA with poly A sequence can be effectively and feasibly purified by oligo dT resin with high purity. The circRNA with poly A sequence is also found to be superior in improving protein expression level and biological function, compared with circRNA counterpart without poly A sequence.

### BRIEF DESCRIPTION OF THE DRAWINGS

FIG. 1 shows schematic representation of DNA template for M12.BBZ linear mRNA (derived from IVT of pDA-M12.BBZ CAR plasmid), M12.BBZ circRNA (derived from IVT of pCA-M12.BBZ CAR plasmid), M12.BBZ-45A circRNA (derived from IVT of pCA45-M12.BBZ CAR plasmid) and M12.BBZ-70A circRNA (derived from IVT of pCA70-M12.BBZ CAR plasmid).

FIG. 2 is process graph showing the purification process of M12.BBZ-45A circRNA by CIMmultus Oligo dT column (s3 represents the flowthrough sample, s4 represents the column wash sample, s5 represents the circRNA elution sample).

FIG. 3 is agarose gel picture showing the bands of different sample collected during the purification process of M12.BBZ-45A circRNA by CIMmultus Oligo dT column; wherein s3 represents the flowthrough sample, s4 represents the column wash sample, s5 represents the M12.BBZ-45A circRNA elution sample.

FIG. 4 is process graph showing the purification process of M12.BBZ-70A circRNA by CIMmultus Oligo dT column (s3 represents the flowthrough sample, s4 represents the circRNA elution sample).

FIG. 5 is agarose gel picture showing the bands of different sample collected during the purification process of M12.BBZ-70A circRNA by CIMmultus Oligo dT column, s3 represents the flowthrough sample, s4 represents the M12.BBZ-70A circRNA elution sample.

FIG. 6 is statistical analysis of the final yield of circRNA purified by oligo dT column.

FIG. 7 shows 24 hours after T cell electroporation, FACS staining and Mean Fluorescence Index (MFI) analysis of Mesothelin-Fc recombinant protein of T cells electroporated with M12.BBZ CAR linear mRNA, M12.BBZ CAR circRNA, M12.BBZ CAR circRNA with 70A. These results show that introducing the poly A sequence into the circRNA can improve the protein expression level of M12.BBZ.

FIG. 8 shows 48 hours after T cell electroporation, FACS staining and Mean Fluorescence Index (MFI) analysis of Mesothelin-Fc recombinant protein of T cells electroporated with M12.BBZ CAR linear mRNA, M12.BBZ CAR circRNA, M12.BBZ CAR circRNA with 70A. These results show that introducing the poly A sequence into the circRNA can improve the protein expression level of M12.BBZ.

FIG. 9 shows the killing curves of CAR-T cells that electroporated with M12.BBZ CAR linear mRNA, M12.BBZ CAR circRNA, M12.BBZ CAR circRNA with 70A to A549 cells electroporated with 5 ug MSLN mRNA at E/T ratio=3:1. These results show that the killing effect of M12.BBZ circRNA with 70A is better than the M12.BBZ circRNA without poly A.

FIG. 10 shows schematic representation of pDA anti-meso CAR-M12 plasmid (i.e., pDA-M12.BBZ CAR plasmid).

FIG. 11 shows schematic representation of pCA-M12.BBZ CAR plasmid.

FIG. 12 shows schematic representation of pCA45-M12.BBZ CAR plasmid.

FIG. 13 shows schematic representation of pCA70-M12.BBZ CAR plasmid.

FIGS. 14A-14B shows the killing curves of CAR-T cells that electroporated with BCMA or FMC63.BBZ CAR linear mRNA, circRNA, circRNA with 70A.

FIGS. 15A-15C shows the killing curves of CAR-T cells that electroporated with M12, BCMA or FMC63.BBZ CAR linear mRNA, circRNA, circRNA with 70A, with or without LACOSTIM expressed.

FIGS. 16A-16C provides the reads of two 96-well plates of anti-human mesothelin (or BCMA and CD123)-Fc monoclonal phage ELISA.

FIGS. 17A-17B provides flow cytometry data of anti-BCMA CAR-T cells stained with CD19-Fc (FIG. 17A) or BCMA-Fc (FIG. 17B).

FIG. 18 provides the exemplary schematic representation of pDA-CAR vector used for CAR mRNA generation.

FIG. 19 provides FACS staining results showing the binding of anti-mesothelin scFv that expressed in CART cells to mesothelin-Fc protein.

FIG. 20A provides FACS staining of the A549 cells that were electroporated with different amount of mesothelin mRNA by anti-mesothelin antibody.

FIG. 20B provides flow cytometry of the A549 cells that were electroporated with different amount of mesothelin mRNA by anti-mesothelin antibody.

FIG. 21 provides the killing curves of different mRNA-based mesothelin CART cells against A549-GFP tumor cells at E/T ratio=10:1.

FIG. 22 provides the killing curves of different mRNA-based mesothelin CART cells against A549-GFP tumor cells that were electroporated with 10 μg mesothelin mRNA at E/T ratio=10:1.

FIG. 23A provides the killing curves of different mRNA-based mesothelin CART cells against A549-GFP tumor cells that were electroporated with 2 μg mesothelin mRNA at E/T ratio=10:1.

FIG. 23B provides the 2^{nd} screening killing curves of different mRNA-based mesothelin CART cells against A549-GFP tumor cells that were electroporated with 0 μg, 2 μg or 10 μg mesothelin mRNA at E/T ratio=10:1 or 3:1.

FIG. 23C provides the killing curves of different mRNA-based mesothelin CART cells against A549-GFP tumor cells that were electroporated with 0 μg, 2 μg or 10 μg mesothelin mRNA at E/T ratio=10:1.

FIG. 24 provides FACS staining of OVCAR3, H226, ASPC1, A549 and HCC70 with isotype control and anti-mesothelin mAb.

FIG. 25A provides the frequencies of CAR+ cells of the T cells transduced with the designated BCMA CARs.

FIG. 25B provides the Medium Fluorescence Intensity (“MFI”) of CAR expression in T cells transduced with the designated BCMA CARs.

FIG. 26 provides the frequencies of CAR+CD8 cells of the T cells transduced with the designated BCMA CARs.

FIG. 27 provides the phenotype of designated CART cells characterized by CCR7 expression and CD45RO expression.

FIG. 28 provides FACS staining results showing the binding of anti-CD123 scFv that expressed in CART cells to CD123-Fc protein.

FIG. 29 provides FACS staining of the A549 cells that were electroporated with different amount of CD123 mRNA with isotype and anti-CD123 antibodies.

FIG. 30 provides the killing curves of different mRNA-based CD123 CART cells against A549-GFP tumor cells at E/T ratio=10:1.

FIG. 31 provides the killing curves of different mRNA-based anti-CD123 CART cells against A549-GFP tumor cells at E/T ratio=3:1.

FIG. 32 provides the killing curves of different mRNA-based anti-CD123 CART cells against A549-GFP tumor cells that were electroporated with 10 μg CD123 mRNA at E/T ratio=10:1.

FIG. 33 provides the killing curves of different mRNA-based anti-CD123 CART cells against A549-GFP tumor cells that were electroporated with 10 μg CD123 mRNA at E/T ratio=3:1.

FIGS. 34A-34C provide diagram of lentiviral vectors constructed (A) and the CAR expression of T cells transduced with the vectors (B). Cytokines production of T cells co-cultured with MSLN CAR and a LACOSTIM (C). Lentiviral vector transduced T cells as indicated were co-cultured with PC3, PC3 transferred with 0.5 ug MSLN RNA (PC3+Meso 0.5 ug) or PC3 transferred with 10 ug MSLN (PC3+Meso 10 ug) (Upper panel), or MSLN positive tumor OVCAR3, H226, or MSLN negative tumor A549 (Lower panel) for 24 h. Supernatant was harvested for cytokine IFNg or IL-2 detection by ELISA.

FIG. 35 provides CD107a staining of different mRNA-based anti-mesothelin CAR-T cells, including mock T cells (NO EP), T cells with LACO (A40C28), anti-mesothelin M12+/−A40C28 CAR-T cells and M32+/−A40C28 CAR-T cells, in the coculture and killing assay with OVCAR3, H226, ASPC1, A549 and HCC70 tumor cell lines.

FIG. 36 provides the killing curves of different mRNA-based anti-mesothelin CAR-T cells, including mock T cells (NO EP), T cells with A40C28, anti-mesothelin M12+/−A40C28 CAR-T cells and M32+/−A40C28 CAR-T cells, to A549-GFP tumor cells that were electroporated with 0, 0.5 μg or 10 μg mesothelin mRNA at E/T ratio=3:1.

FIG. 37 provides the killing curves of lentivirus-based anti-mesothelin CAR-T cells, including mock T cells (UTD) and anti-mesothelin M12+/−A40C28 CAR-T cells, to H226, OVCAR3 and MOLM14 cells that were electroporated with 0 or 10 μg mesothelin mRNA at E/T ratio=2:1.

FIG. 38 provides the killing curves of different mRNA-based anti-mesothelin CAR-T cells, including mock T cells (NO EP), anti-mesothelin M12+/−1412-4D11 CAR-T cells and M32+/−1412-4D11 CAR-T cells, to A549-GFP tumor cells that were electroporated with 0 or 2 μg mesothelin mRNA at E/T ratio=10:1.

FIG. 39 provides the lytic activity of lentiviral transduced T cells as indicated against MLSN negative tumor PC3, MOLM14.

FIG. 40 provides FACS staining of OVCAR3, H226, ASPC1, A549, HCC70, 786-0 and Jeko1 cells with isotype control, anti-mesothelin mAb and anti-CD40 mAb.

FIG. 41 provides CD107a staining of different mRNA-based anti-mesothelin CAR-T cells, including mock T cells (NO EP), T cells with A40C28 alone, T cells with 1412-4D11 alone, anti-mesothelin M12+/−A40C28 or 1412-4D11 CAR-T cells and M32+/−A40C28 or 1412-4D11 CAR-T cells, in the coculture and killing assay with OVCAR3, H226, ASPC1, A549, HCC70, 786-0 and Jeko1 tumor cell lines.

FIG. 42 provides the killing curves of different mRNA-based anti-mesothelin CAR-T cells, including mock T cells (NO EP), anti-mesothelin M12+/−A40C28 or 1412-4D11 CAR-T cells and M32+/−A40C28 or 1412-4D11 CAR-T cells, to OVCAR3-GFP, H226-GFP and ASPC1 tumor cells at E/T ratio=3:1.

FIG. 43 provides ELISA results of CART killing assay measuring the IFN-γ and IL2 release. Different mRNA-based anti-mesothelin CAR-T cells, including mock T cells (NO EP), anti-mesothelin M12+/−A40C28 or 1412-4D11 CAR-T cells and M32+/−A40C28 or 1412-4D11 CAR-T cells, were cocultured with OVCAR3-GFP, H226-GFP and ASPC1 tumor cells at E/T ratio=1:1.

FIGS. 44A-44B provide the MSLN and CD40 expression of different tumor cell lines (A). CD137 expression of lentiviral transduced T cells as indicated stimulated with a panel of tumor cell line expressing MLSN at different levels (B).

FIGS. 45A-45D provide the anti-tumor efficacy of A40C28-M12 LVV CART in H226 xenograft NOG tumor mode. The timeline of CART treatment in H226 xenograft NSG tumor model (A). UTD, M12, CAR or LACOSTIM expression of lentiviral transduced T cells by measuring the level of surface CAR (stained with MSLN-Fc) and LACOSTIM (stained with CD40-Fc) expression using flow cytometry analysis (B). NSG mice were implanted with 5E6 of H226-CBG cells and 11 days later treated with 1E6 or 5E6 of CAR positive T cells (M12, A40C28-M12) or non-transduced T cells (UTD) or T cell transduced only A40C28 (A40C28) as controls. Tumor size (C) and Bioluminescence (BLI) (D) were measured at multiple time points post-infusion as indicated.

FIGS. 46A-46C provide the TIL analysis in A40C28-M12 treated H226-xenograft model. TILs were separated by enzymatic digestion with 1 mg/ml Collagenase and 30 U/ml DNase I in RPIM 1640 at 37° C. with rotation for 1.5 hours (A). Schematic representation of multiplexed immunohistochemical (mIHC) (B). Distribution of CD4+ T cell, CD8+ T cell, Granzyme B+ T cell and MSLN+ target cells in tumor tissue were detected by multiplexed immunohistochemical (mIHC) (C).

FIGS. 47A-47B provide the expression of BCMA in tumor lines. FIG. 47A provides the FACS results. FIG. 47B provides the relative expression levels as compared to A549 cells.

FIGS. 48A-48B provide ELISA results showing the production of INF-γ and IL-2 by designated CART cells FIG. 48A shows INF-γ production. FIG. 48B shows IL-2 production.

FIGS. 49A-49D provide results of the tumor killing assay showing the cytolytic activities of designated CART cells against Jeko-1 cells at different E (T cells):T (tumor cells) ratio. FIG. 49A: E:T=0.1:1; FIG. 49B: E:T=0.5:1; FIG. 49C: E:T=2:1; FIG. 49D: E:T=2:1 (enlarged view).

FIGS. 50A-50E provide results of the tumor killing assay showing the cytolytic activities of designated CART cells against RPMI-8226 cells. FIG. 50A: E:T=0.1:1; FIG. 50B: E:T=0.5:1;

FIG. 50C: E:T=0.5:1 (enlarged view); FIG. 50D: E:T=2:1; FIG. 50D: E:T=2:1 (enlarged view).

FIGS. 51A-51B provide the FACS results showing the expression levels of CAR and LACO in the T cells (FIG. 51A) and the MFI of CAR (FIG. 51B).

FIGS. 52A-52B provide results showing the numbers (FIG. 52A) and the sizes of designated CART cells (FIG. 52B) during culture.

FIGS. 53A-53B provide ELISA results showing cytokine production by T cells after coculture with a panel of tumor cells. FIG. 53A shows IL-2 production. FIG. 53B shows INF-γ production.

FIGS. 54A-54C provides results of in vivo animal experiment comparing the killing effects of BCMA31, LACO-BCMA31, BCMA31-LACO, and B38M CART cells against Jeko-1 tumor cells. FIG. 54A shows the bioluminescence imaging of Jeko-1 tumors. FIG. 54B shows the average Radiance of bioluminescence. FIG. 54C shows the survival of the mice.

FIG. 55 provides FACS results showing the expression levels of CAR and LACO in the T cells after mRNA electroporation.

FIG. 56 provides the expression of CD107a in designated CART cells after coculture with different tumor cells.

FIGS. 57A-57D provide results of Incucyte Live-Cell Analysis of the cytotoxic T cell activities of designated CART cells against different tumor cells. FIG. 57A: Nalm6 cells;

FIG. 57B: Jeko-1 cells; FIG. 57C: RPMI-8226 cells; FIG. 57D: Raji cells.

FIG. 58 shows FACS staining of A549, SK-OV3, Jeko-1, Molm-14, SupT-1, 293T, Nalm-6 and PC-3 cells with PE-isotype control and PE-anti-CD123 mAb.

FIG. 59 shows CD107a staining of anti-CD123-C5, anti-CD123-C7, anti-CD123-C11 CART cells in the coculture and killing assay with different tumor cell lines.

FIG. 60 provides the killing curves of different mRNA-based anti-CD123 CART cells with or without LACO (A40C.CD28) against MOLM-14, NALM6 or JEKO-1 tumor cells at E/T ratio=10:1

FIG. 61 provides the killing curves of different mRNA-based anti-CD123 CART cells with or without LACO (A40C.CD28) against A549 tumor cells that were electroporated with 10 μg, 0.1 μg or 0 CD123 mRNA at E/T ratio=30:1.

FIG. 62 provides ELISA results showing the IFN-gamma secretion of the T cells electroporated with different CD123 CAR with or without LACO.

FIG. 63 provides results of five representative 96-well plate of anti-human CD40-Fc monoclonal phage ELISA. Colony 18 #, 37 #, 38 #, 45 #, 47 # and 52 # produced the scFv(s) designated as 40-18, 40-37, 40-38, 40-45, 40-47, and 40-52, which were selected for further studies.

FIG. 64 provides FACS staining results showing the binding of the anti-CD40 scFv(s) expressed in CAR-T cells to CD40-Fc protein.

FIG. 65 provides the killing curves of different mRNA-based CD40 scFv+anti-Her2 CART cells against A549-GFP tumor cells at different E/T ratio.

FIGS. 66A-66C provide CD107a staining of CART cells in the coculture and killing assay with A549 cells (FIG. 66A), PC-3 (FIG. 66B) and SK-OV3 (FIG. 66C).

### DETAILED DESCRIPTION

It should be understood that this invention is not limited to particular embodiments described herein. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are to disclose and describe the methods and/or materials in connection with which the publications are cited.

Where a range of values with one or two limits is provided, it is understood that a smaller range between any stated intervening value in that stated range and either limit of that stated range is encompassed within the invention. Where the stated range includes one or two limits, ranges excluding either or both of the limits are also included in the invention.

### Terminology

It must be noted that as used herein and in the appended claims, the singular forms “a,” “an,” and “the” include plural referents unless the context clearly dictates otherwise.

It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Unless otherwise stated, the term “comprise”, “include”, “contain” and variations of these terms, such as comprising, comprises and comprised, are not intended to exclude further members, components, integers or steps. These terms also encompass the meaning of “consist of” or “consisting of”. The term “consist of” or “consisting of” is a particular embodiment of the term “comprise”, wherein any other non-stated member, component, integer or step is excluded.

The term “about” refers to a range equal to the particular value plus or minus ten percent (+/−10%).

The term “and/or” refers to any one, several or all of the elements connected by the term.

The terms “circRNA”, “circular RNA” or “cRNA”, as used herein, refers to a RNA molecule that forms a circular structure through covalent bonds.

The term “internal ribosome entry site (IRES)”, as used herein refers to an RNA sequence capable of initiating translation of a polypeptide in the absence of a typical RNA cap structure.

The term “vector”, as used herein, refers to a piece of DNA, that is synthesized (e.g., using PCR), or that is taken from a virus, plasmid, or cell of a higher organism into which a foreign DNA fragment can be or has been inserted for cloning and/or expression purposes. A vector can be used for inducing a nucleic acid into a cell. A vector can be stably maintained in a cell or an organism. A vector may comprise, for example, an origin of replication, a selectable marker or reporter gene, such as antibiotic resistance or fluorescent protein gene, and/or a multiple cloning site (MCS). The term “vector” includes linear vector or a circular vector, such as linear DNA fragments (e.g., PCR products, linearized plasmid fragments), plasmid vectors, viral vectors, cosmids, bacterial artificial chromosomes (BACs), yeast artificial chromosomes (YACs), and the like.

The term “element”, as used herein, refers to a separate or distinct part of something, for example, a nucleic acid sequence with a separate function within a longer nucleic acid sequence.

The term “operably linked”, as used herein, refers to the association of nucleic acid sequences on a single nucleic acid fragment so that the function of one is affected by the other.

For example, a promoter is operably linked with a coding sequence when it is capable of affecting the expression of that coding sequence (i.e., that the coding sequence is under the transcriptional control of the promoter). In the present invention, the term “operably linked” means that the elements of a vector are positioned such that they can be transcribed to form a precursor RNA, the elements of a precursor RNA are positioned such that they can then be circularized into a circular RNA and/or the elements of a circular RNA are positioned such that they can be translated to produce a protein.

The term “adjacent” and its grammatical equivalents as used herein refers to right next to the object of reference. For example, the term “adjacent” in the context of a nucleotide sequence can mean without any nucleotides in between, i.e., the absence of intervening sequences between two nucleotide sequences.

The term “sequence identity”, as used herein, refers to the percentage of identical nucleotide or amino acid residues at corresponding positions in two or more sequences when the sequences are aligned to maximize sequence matching, i.e., taking into account gaps and insertions. The alignment of the sequences and the calculation of percentage of the sequence identity can be carried out with suitable computer programs known in the art. Such programs include, but are not limited to, BLAST, ALIGN, ClustalW, EMBOSS Needle, etc. An example of a local alignment program is BLAST (Basic Local Alignment Search Tool), which is available from the webpage of National Center for Biotechnology Information which can currently be found at http://www.ncbi.nlm.nih.gov// and which was firstly described in Altschul et al. (1990) J. Mol. Biol. 215; 403-410. Examples of a global alignment program (which optimizes the alignment over the full-length of the sequences) are EMBOSS Needle and EMBOSS Stretcher programs based on the Needleman-Wunsch algorithm (Needleman, Saul B.; and Wunsch, Christian D. (1970), “A general method applicable to the search for similarities in the amino acid sequence of two proteins”, Journal of Molecular Biology 48 (3): 443-53), which are both available at http://www.ebi.ac.uk/Tools/psa/.

The term “antibody”, as used herein, refers to an immunoglobulin, antigen-binding fragment, or derivative thereof, that specifically binds to and recognizes an antigen, an antigenic fragment thereof, or a dimer or multimer of the antigen. The term “antibody” is used herein in the broadest sense and encompasses various antibody structures, including but not limited to monoclonal antibodies, polyclonal antibodies, multispecific antibodies (e.g., bispecific antibodies), and antibody fragments, so long as they exhibit the desired antigen-binding activity. Non-limiting examples of antibodies include, for example, intact immunoglobulins and variants and fragments thereof that retain binding affinity for the antigen. Examples of antibody fragments include but are not limited to Fv, Fab, Fab′, Fab′-SH, F(ab′)₂; diabodies; linear antibodies; single-chain antibody molecules (e.g., scFv); single domain antibody (VHH); and multispecific antibodies formed from antibody fragments. Antibody fragments include antigen binding fragments either produced by the modification of whole antibodies or those synthesized de novo using recombinant DNA methodologies (see, e.g., Kontermann and Dubel (Ed), Antibody Engineering, Vols. 1-2, 2^{nd} Ed., Springer Press, 2010).

A classical full-length antibody molecule is an immunoglobulin molecule (e.g., IgG) or its multimers (e.g. IgA or IgM) composed of four polypeptide chains. The four polypeptide chains include two identical heavy chains (H) and two identical light chains (L), which are linker by a disulfide bond to form a tetramer. Each heavy chain consists of a heavy chain variable region (“HCVR” or “VH”) and a heavy chain constant region (CH, including the structural domains CH1, CH2 and CH3). Each light chain consists of a light chain variable region (“LCVR” or “VL”) and a light chain constant region (CL). The heavy chain and the constant region of the light chain (CH and CL) are not directly involved in antibody-antigen binding, but exhibit a variety of effector functions, such as mediating antibody binding to tissues or factors of the host, including various cells of the immune system (e.g., effector cells) and the first component of the classical complement system (C1q). The variable regions of the heavy and light chains (VH and VL) form the antigen-binding site. VH and VL each have highly variable regions known as complementarity determining regions (CDR) with a high degree of variability in amino acid composition and sequence arrangement, which are critical sites for antibody-antigen binding, interspersed with more conserved sequences known as framework regions (FR). Each VH and VL consists of three CDRs and four FRs arranged in the following order from the amino terminus to the hydroxyl terminus: FR1, CDR1, FR2, CDR2, FR3, CDR3, FR4. In this paper, the three heavy chain complementary determining regions may also be called HCDR1, HCDR2 and HCDR3, respectively. The four heavy chain framework regions are called HFR1, HFR2, HFR3 and HFR4, respectively; the three light chain complementary decision regions may also be called LCDR1, LCDR2 and LCDR3, respectively, and the four light chain framework regions are called LFR1, LFR2, LFR3, and LFR4, respectively. The variable regions of the heavy and light chains (VH and VL) form the antigen binding site, respectively.

As used herein, the term “complementary determining region” or “CDR” refers to the amino acid residues responsible for antigen binding in the variable region of the antibody. The precise boundaries of the CDR can be defined according to various numbering systems known in the art, for example, according to the Kabat numbering system (Kabat et al., Sequences of Proteins of Immunological Interest, 5th Ed. Public Health Service, National Institutes of Health, Bethesda, Md., 1991), the Chothia numbering system (Chothia & Lesk (1987) J. Mol. Biol. 196:901-917; Chothia et al: 878-883) or as defined in the IMGT numbering system (Lefranc et al., Dev. Comparat. Immunol. 27:55-77, 2003). For a given antibody, a person skilled in the art will readily identify the CDR as defined according to the respective numbering system, and the correspondence between the different numbering systems is well known to a person skilled in the art (see, for example, Lefranc et al., Dev. Comparat. Immunol. 27:55-77, 2003). The antibodies of the present invention may utilize any of these numbering systems to define the CDR, although it is preferred that the Kabat numbering system be used to define the CDR.

The term “chimeric antigen receptor” or “CAR” refers to a fusion protein comprising an extracellular domain capable of binding to an antigen (i.e., binding domain), a transmembrane domain and an intracellular domain comprising one or more intracellular signaling domains derived from signal transducing proteins. These intracellular signaling domains are typically different from the polypeptide from which the extracellular domain is derived. The extracellular domain can be any proteinaceous molecule or part thereof that can specifically bind to a predetermined antigen. In some embodiments, the extracellular domain comprises an antibody or antigen binding fragment thereof. In some embodiments, the intracellular signaling domain can be any oligopeptide or polypeptide domain known to function to transmit a signal causing activation or inhibition of a biological process in a cell, for example, activation of an immune cell such as a T cell or a NK cell. Intracellular signaling domains typically include immunoreceptor tyrosine activation motifs (ITAM), such as signaling domains derived from CD3ζ molecules, responsible for activating immune effector cells and producing killing effects. Alternatively, chimeric antigen receptors may also include a signaling peptide at the amino terminus responsible for intracellular localization of the nascent protein, as well as a hinge region between the extracellular domain and the transmembrane domain. The intracellular signaling domain may also include a co-stimulatory structural domain derived from, for example, 4-1BB or CD28 molecules.

The term “purify”, “purifying” or “purification”, as used herein, generally refers to isolation of the substance of interest (for example, a compound, a polynucleotide, a protein or a polypeptide) such that the substance constitutes the main component of the purified product, such as 70% or more, 80% or more, 90% or more, 91% or more, 92% or more, 93% or more, 94% or more, 95% or more, 96% or more, 97% or more, 98% or more, 99% or more or 100% of the purified product.

The term “transcribe”, “transcribing” or “transcription”, as used herein, means the formation or synthesis of an RNA molecule by an RNA polymerase using a DNA molecule as a template. The RNA polymerase that can be used in the present invention includes, but is not limited to, T7-type RNA polymerase.

The term “translate”, “translating” or “translation”, as used herein, means the formation of a polypeptide molecule by a ribosome based upon an RNA template.

The term “treat”, “treating” or “treatment”, as used herein, refers to provide a beneficial or desired clinical outcome to a disease, such as eliminating the disease, alleviating the symptoms, diminishing the extent of the disease, stabilizing, ameliorating or palliating the state of the disease, or slowing the progress of a disease. Measurement of the treatment outcome may be based on, e.g., the results of a physical examination, a pathological test and/or a diagnostic test as known in the art. Treatment may also mean prolonging survival as compared to expected survival if a subject was not receiving treatment. Treatment may also refer to reducing the incidence or onset of a disease, or a recurrence thereof, as compared to that which would occur in the absence of the measure taken. Clinically, such a treatment can also be called prevention.

The term “pharmacologically acceptable carrier”, as used herein, refers to any carrier that is comprised in a pharmaceutical composition as a non-active ingredient that allows the pharmaceutical composition to have an appearance and properties suitable for administration. The pharmacologically acceptable carrier has substantially no long term or permanent detrimental effect when administered to a subject, such as a stabilizer, diluent, additive, auxiliary, excipient and the like. “Pharmaceutically acceptable carrier” should be a pharmaceutically inert material that has substantially no biological activity and constitutes a substantial part of the formulation.

The terms “subject” and “patient” may be used interchangeably herein. The term “subject”, as used herein, refers to any organism to which the active agent of the composition of the present invention may be administered, e.g., for experimental, diagnostic, prophylactic, and/or therapeutic purposes. Typical subjects include animals (e.g., mammals such as mice, rats, rabbits, non-human primates such as chimpanzees and other apes and monkey species, and humans). The subject may be a mammal, particularly a human, including a male or female, and including a neonatal, infant, juvenile, adolescent, adult or geriatric, and further is inclusive of various races and ethnicities.

The terms “therapeutically effective amount” and “effective amount”, as used herein, can be used interchangeably and refer to an amount effective, at dosages and for periods of time necessary, to achieve a desired therapeutic result. A therapeutically effective amount may vary according to factors such as the disease state, age, sex, and weight of the individual, and the ability of a therapeutic or a combination of therapeutics to elicit a desired response in the individual. An “effective amount” can designate an amount that causes a detectable change in biological or chemical activity. The detectable changes may be detected and/or further quantified by one skilled in the art for the relevant mechanism or process. Moreover, an “effective amount” can designate an amount that maintains a desired physiological state, i.e., reduces or prevents significant decline and/or promotes improvement in the condition.

Circular RNA, the Preparation and the Use Thereof

Circular RNA (circRNA) has been artificially constructed to express proteins. Such circular RNA generally comprises IRES and protein coding sequence.

In the present invention, we introduce poly A sequence into the circRNA molecule. The circRNA with poly A sequence can be effectively and feasibly purified by oligo dT resin with high purity. The circRNA with poly A sequence is also found to be superior in improving protein expression level and biological function, compared with circRNA counterpart without poly A sequence.

In some embodiments, the present invention relates to a circular RNA, the circular RNA comprising, in the following order, a IRES element, a protein coding sequence and a polyA, i.e., the IRES element is positioned on 5′ of the protein coding sequence and the polyA is positioned on 3′ of the protein coding sequence.

The IRES may derived from a virus. The IRES may be generally about 10 nt to 1000 nt or more in length, typically about 500 nt to about 1000 nt in length. In some embodiments, the IRES sequence is an IRES sequence from Coxsackievirus B3 (CVB3) or Coxsackievirus A (CVB1/2), Encephalomyocarditis virus (EMCV), Taura syndrome virus, Triatoma virus, Theiler's encephalomyelitis virus, simian Virus 40, Solenopsis invicta virus 1, Rhopalosiphum padi virus, Reticuloendotheliosis virus, fuman poliovirus 1, Plautia stali intestine virus, Kashmir bee virus, Human rhinovirus 2, Homalodisca coagulata virus-1, Human Immunodeficiency Virus type 1, Homalodisca coagulata virus-1, Himetobi P virus, Hepatitis C virus, Hepatitis A virus, Hepatitis GB virus, foot and mouth disease virus, Human enterovirus 71, Equine rhinitis virus, Ectropis obliqua picorna-like virus, Drosophila C Virus, Crucifer tobamo virus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPA1, Human AMLURUNX1, Drosophila antennapedia, Human AQP4, Human AT1R, Human BAG-1, Human BCL2, Human BiP, Human c-IAP1, Human c-myc, Human eIF4G, Mouse NDST4L, Human LEF1, Mouse HIF1 alpha, Human n.myc, Mouse Gtx, Human p27kipl, Human PDGF2/c-sis, Human p53, Human Pim-1, Mouse Rbm3, Drosophila reaper, Canine Scamper, Drosophila Ubx, Human UNR, Mouse UtrA, Human VEGF-A, Human XIAP, Salivirus, Cosavirus, Parechovirus, Drosophila hairless, S. cerevisiae TFIID, S. cerevisiae YAP1, Human c-src, Human FGF-1, Simian picomavirus, Turnip crinkle virus, an aptamer to eIF4G. In some embodiments, the IRES is an IRES sequence of Coxsackievirus B3 (CVB3).

The protein coding sequence may encode one or more proteins. The protein coding sequence may encode a protein of eukaryotic or prokaryotic origin. The protein coding sequence may encode human protein or non-human protein. The protein coding sequence may encode a protein for therapeutic use. In some embodiments, the protein may be an antibody, an antigen, a cytokine, an enzyme, a fluorescent protein, a chimeric antigen receptor (CAR), a T cell receptor (TCR), or a fusion protein comprising an antibody, an antigen, a cytokine, an enzyme or a fluorescent protein.

The term “therapeutic protein”, as used herein, refers to any protein that has a therapeutic, diagnostic, and/or prophylactic effect and/or elicits a desired biological and/or pharmacological effect when administered to a subject.

In some embodiments, the CAR comprises a binding domain which can specifically bind a tumor antigen, such as mesothelin. The binding domain may comprise an antibody or a ligand to a tumor antigen or a tumor surface receptor. In some embodiments, the tumor antigen or the tumor surface receptor may be mesothelin. In some embodiments, the binding domain may comprise, e.g., anti-mesothelin scFv. In some embodiments, the light chain variable region of the antibody against mesothelin comprises LCDR1, LCDR2, and LCDR3 as set forth in SEQ ID NOs: 576-578 respectively. In some embodiments, the heavy chain variable region of the antibody against mesothelin comprises HCDR1, HCDR2, and HCDR3 as set forth in SEQ ID NOs: 579-581 respectively. In some embodiments, the light chain variable region of the antibody against mesothelin comprises the sequence as set forth in SEQ ID NO: 574. In some embodiments, the heavy chain variable region of the antibody against mesothelin comprises the sequence as set forth in SEQ ID NO: 575. In some embodiments, the anti-mesothelin scFv comprises a sequence as set forth in SEQ ID NO: 598. In some embodiments, the CAR comprises a sequence as set forth in SEQ ID NO: 599.

In some embodiments, the protein encoded by the protein coding sequence comprises an antibody or a CAR comprising the antibody as a binding domain, wherein the antibody specifically binds to TSHR, CD19; CD123; CD22; CD30; CD171; CS-1; C-type lectin-like molecule-1, CD33; epidermal growth factor receptor variant III (EGFRvIII); ganglioside G2 (GD2); ganglioside GD3; TNF receptor family member; B-cell maturation antigen; Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2; Mesothelin; Interleukin 11 receptor alpha (IL-11Ra); prostate stem cell antigen (PSCA); Protease Serine 21; vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gplOO); oncogene polypeptide consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3; transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WTl); Cancer/testis antigen 1 (NY-ESO-1); Cancer/testis antigen 2 (LAGE-la); Melanoma-associated antigen 1 (MAGE-A1); ETS translocation-variant gene 6, located on chromosome 12p (ETV6-AML); sperm protein 17 (SPA 17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1, melanoma antigen recognized by T cells 1; Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B 1 (CYP1B1); CCCTC-Binding Factor (Zinc Finger Protein)-Like, Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIRI); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

In some embodiments, the antibody specifically binds to mesothelin, CD123, BCMA, HER2, IL13Ra2, B7H3 or CD40. In some embodiments, the antibody is a scFv. In some embodiments, the scFv comprises a heavy chain variable region (VH) fused to N-terminal or C-terminal of a light chain variable region (VL). In some embodiments, an amino acid linker may be positioned between the VH and VL in the scFv.

In some embodiments, the antibody specifically binds to mesothelin, which is also called anti-mesothelin (or anti-MESO or anti-MSLN) antibody, is the antibody described in PCT/CN2022/112726 (which is incorporated herein by reference in its entirety), including a light chain variable region comprising LCDR1, LCDR2 and LCDR3 and a heavy chain variable region comprising HCDR1, HCDR2, and HCDR3, wherein LCDR1, LCDR2, LCDR3, HCDR1, HCDR2, and HCDR3 are selected from the following group:
(1) a LCDR1 as set forth in SEQ ID NO: 1, a LCDR2 as set forth in SEQ ID NO: 16, a LCDR3 as set forth in SEQ ID NO: 30, a HCDR1 as set forth in SEQ ID NO: 45, a HCDR2 as set forth in SEQ ID NO: 58 and a HCDR3 as set forth in SEQ ID NO: 71;
(2) a LCDR1 as set forth in SEQ ID NO: 2, a LCDR2 as set forth in SEQ ID NO: 17, a LCDR3 as set forth in SEQ ID NO: 31, a HCDR1 as set forth in SEQ ID NO: 46, a HCDR2 as set forth in SEQ ID NO: 59 and a HCDR3 as set forth in SEQ ID NO: 72;
(3) a LCDR1 as set forth in SEQ ID NO: 3, a LCDR2 as set forth in SEQ ID NO: 18, a LCDR3 as set forth in SEQ ID NO: 32, a HCDR1 as set forth in SEQ ID NO: 47, a HCDR2 as set forth in SEQ ID NO: 60 and a HCDR3 as set forth in SEQ ID NO: 73;
(4) a LCDR1 as set forth in SEQ ID NO: 4, a LCDR2 as set forth in SEQ ID NO: 19, a LCDR3 as set forth in SEQ ID NO: 33, a HCDR1 as set forth in SEQ ID NO: 48, a HCDR2 as set forth in SEQ ID NO: 61 and a HCDR3 as set forth in SEQ ID NO: 74;
(5) a LCDR1 as set forth in SEQ ID NO: 5, a LCDR2 as set forth in SEQ ID NO: 20, a LCDR3 as set forth in SEQ ID NO: 34, a HCDR1 as set forth in SEQ ID NO: 49, a HCDR2 as set forth in SEQ ID NO: 62 and a HCDR3 as set forth in SEQ ID NO: 75;
(6) a LCDR1 as set forth in SEQ ID NO: 6, a LCDR2 as set forth in SEQ ID NO: 21, a LCDR3 as set forth in SEQ ID NO: 35, a HCDR1 as set forth in SEQ ID NO: 50, a HCDR2 as set forth in SEQ ID NO: 63 and a HCDR3 as set forth in SEQ ID NO: 76;
(7) a LCDR1 as set forth in SEQ ID NO: 7, a LCDR2 as set forth in SEQ ID NO: 22, a LCDR3 as set forth in SEQ ID NO: 36, a HCDR1 as set forth in SEQ ID NO: 51, a HCDR2 as set forth in SEQ ID NO: 64 and a HCDR3 as set forth in SEQ ID NO: 77;
(8) a LCDR1 as set forth in SEQ ID NO: 8, a LCDR2 as set forth in SEQ ID NO: 23, a LCDR3 as set forth in SEQ ID NO: 37, a HCDR1 as set forth in SEQ ID NO: 52, a HCDR2 as set forth in SEQ ID NO: 65 and a HCDR3 as set forth in SEQ ID NO: 78;
(9) a LCDR1 as set forth in SEQ ID NO: 9, a LCDR2 as set forth in SEQ ID NO: 24, a LCDR3 as set forth in SEQ ID NO: 38, a HCDR1 as set forth in SEQ ID NO: 53, a HCDR2 as set forth in SEQ ID NO: 66 and a HCDR3 as set forth in SEQ ID NO: 79;
(10) a LCDR1 as set forth in SEQ ID NO: 10, a LCDR2 as set forth in SEQ ID NO: 25, a LCDR3 as set forth in SEQ ID NO: 39, a HCDR1 as set forth in SEQ ID NO: 48, a HCDR2 as set forth in SEQ ID NO: 61 and a HCDR3 as set forth in SEQ ID NO: 80;
(11) a LCDR1 as set forth in SEQ ID NO: 11, a LCDR2 as set forth in SEQ ID NO: 26, a LCDR3 as set forth in SEQ ID NO: 40, a HCDR1 as set forth in SEQ ID NO: 54, a HCDR2 as set forth in SEQ ID NO: 67 and a HCDR3 as set forth in SEQ ID NO: 81;
(12) a LCDR1 as set forth in SEQ ID NO: 12, a LCDR2 as set forth in SEQ ID NO: 27, a LCDR3 as set forth in SEQ ID NO: 41, a HCDR1 as set forth in SEQ ID NO: 53, a HCDR2 as set forth in SEQ ID NO: 66 and a HCDR3 as set forth in SEQ ID NO: 82;
(13) a LCDR1 as set forth in SEQ ID NO: 13, a LCDR2 as set forth in SEQ ID NO: 28, a LCDR3 as set forth in SEQ ID NO: 42, a HCDR1 as set forth in SEQ ID NO: 55, a HCDR2 as set forth in SEQ ID NO: 68 and a HCDR3 as set forth in SEQ ID NO: 83;
(14) a LCDR1 as set forth in SEQ ID NO: 14, a LCDR2 as set forth in SEQ ID NO: 29, a LCDR3 as set forth in SEQ ID NO: 43, a HCDR1 as set forth in SEQ ID NO: 56, a HCDR2 as set forth in SEQ ID NO: 69 and a HCDR3 as set forth in SEQ ID NO: 84; and
(15) a LCDR1 as set forth in SEQ ID NO: 15, a LCDR2 as set forth in SEQ ID NO: 18, a LCDR3 as set forth in SEQ ID NO: 44, a HCDR1 as set forth in SEQ ID NO: 57, a HCDR2 as set forth in SEQ ID NO: 70 and a HCDR3 as set forth in SEQ ID NO: 85.

In some embodiments, the anti-MESO antibody comprises a light chain variable region and a heavy chain variable region selected from the following group:
(1) a light chain variable region as set forth in SEQ ID NO: 86 and a heavy chain variable region as set forth in SEQ ID NO: 101;
(2) a light chain variable region as set forth in SEQ ID NO: 87 and a heavy chain variable region as set forth in SEQ ID NO: 102;
(3) a light chain variable region as set forth in SEQ ID NO: 88 and a heavy chain variable region as set forth in SEQ ID NO: 103;
(4) a light chain variable region as set forth in SEQ ID NO: 89 and a heavy chain variable region as set forth in SEQ ID NO: 104;
(5) a light chain variable region as set forth in SEQ ID NO: 90 and a heavy chain variable region as set forth in SEQ ID NO: 105;
(6) a light chain variable region as set forth in SEQ ID NO: 91 and a heavy chain variable region as set forth in SEQ ID NO: 106;
(7) a light chain variable region as set forth in SEQ ID NO: 92 and a heavy chain variable region as set forth in SEQ ID NO: 107;
(8) a light chain variable region as set forth in SEQ ID NO: 93 and a heavy chain variable region as set forth in SEQ ID NO: 108;
(9) a light chain variable region as set forth in SEQ ID NO: 94 and a heavy chain variable region as set forth in SEQ ID NO: 109;
(10) a light chain variable region as set forth in SEQ ID NO: 95 and a heavy chain variable region as set forth in SEQ ID NO: 100;
(11) a light chain variable region as set forth in SEQ ID NO: 96 and a heavy chain variable region as set forth in SEQ ID NO: 111;
(12) a light chain variable region as set forth in SEQ ID NO: 97 and a heavy chain variable region as set forth in SEQ ID NO: 112;
(13) a light chain variable region as set forth in SEQ ID NO: 98 and a heavy chain variable region as set forth in SEQ ID NO: 113;
(14) a light chain variable region as set forth in SEQ ID NO: 99 and a heavy chain variable region as set forth in SEQ ID NO: 114; and
(15) a light chain variable region as set forth in SEQ ID NO: 100 and a heavy chain variable region as set forth in SEQ ID NO: 115.

In some embodiments, the anti-MESO antibody is an anti-MESO scFv, which may comprise an amino acid sequence selected from SEQ ID NOs: 116-130.

In some embodiments, the antibody specifically binds to CD123, which is also called anti-CD123 antibody, is the antibody described in PCT/CN2022/112724 (which is incorporated herein by reference in its entirety), including a light chain variable region comprising LCDR1, LCDR2 and LCDR3 and a heavy chain variable region comprising HCDR1, HCDR2, and HCDR3, wherein LCDR1, LCDR2, LCDR3, HCDR1, HCDR2, and HCDR3 are selected from the following group:
(1) a LCDR1 as set forth in SEQ ID NO: 263, a LCDR2 as set forth in SEQ ID NO: 293, a LCDR3 as set forth in SEQ ID NO: 321, a HCDR1 as set forth in SEQ ID NO: 351, a HCDR2 as set forth in SEQ ID NO: 372 and a HCDR3 as set forth in SEQ ID NO: 396;
(2) a LCDR1 as set forth in SEQ ID NO: 277, a LCDR2 as set forth in SEQ ID NO: 305, a LCDR3 as set forth in SEQ ID NO: 335, a HCDR1 as set forth in SEQ ID NO: 349, a HCDR2 as set forth in SEQ ID NO: 382 and a HCDR3 as set forth in SEQ ID NO: 409;
(3) a LCDR1 as set forth in SEQ ID NO: 267, a LCDR2 as set forth in SEQ ID NO: 297, a LCDR3 as set forth in SEQ ID NO: 326, a HCDR1 as set forth in SEQ ID NO: 355, a HCDR2 as set forth in SEQ ID NO: 376 and a HCDR3 as set forth in SEQ ID NO: 400;
(4) a LCDR1 as set forth in SEQ ID NO: 278, a LCDR2 as set forth in SEQ ID NO: 292, a LCDR3 as set forth in SEQ ID NO: 336, a HCDR1 as set forth in SEQ ID NO: 360, a HCDR2 as set forth in SEQ ID NO: 383 and a HCDR3 as set forth in SEQ ID NO: 410;
(5) a LCDR1 as set forth in SEQ ID NO: 283, a LCDR2 as set forth in SEQ ID NO: 310, a LCDR3 as set forth in SEQ ID NO: 342, a HCDR1 as set forth in SEQ ID NO: 359, a HCDR2 as set forth in SEQ ID NO: 389 and a HCDR3 as set forth in SEQ ID NO: 417;
(6) a LCDR1 as set forth in SEQ ID NO: 284, a LCDR2 as set forth in SEQ ID NO: 311, a LCDR3 as set forth in SEQ ID NO: 343, a HCDR1 as set forth in SEQ ID NO: 366, a HCDR2 as set forth in SEQ ID NO: 388 and a HCDR3 as set forth in SEQ ID NO: 418;
(7) a LCDR1 as set forth in SEQ ID NO: 268, a LCDR2 as set forth in SEQ ID NO: 298, a LCDR3 as set forth in SEQ ID NO: 327, a HCDR1 as set forth in SEQ ID NO: 356, a HCDR2 as set forth in SEQ ID NO: 377 and a HCDR3 as set forth in SEQ ID NO: 401;
(8) a LCDR1 as set forth in SEQ ID NO: 264, a LCDR2 as set forth in SEQ ID NO: 294, a LCDR3 as set forth in SEQ ID NO: 322, a HCDR1 as set forth in SEQ ID NO: 349, a HCDR2 as set forth in SEQ ID NO: 370 and a HCDR3 as set forth in SEQ ID NO: 394;
(9) a LCDR1 as set forth in SEQ ID NO: 285, a LCDR2 as set forth in SEQ ID NO: 312, a LCDR3 as set forth in SEQ ID NO: 344, a HCDR1 as set forth in SEQ ID NO: 367, a HCDR2 as set forth in SEQ ID NO: 390 and a HCDR3 as set forth in SEQ ID NO: 419;
(10) a LCDR1 as set forth in SEQ ID NO: 286, a LCDR2 as set forth in SEQ ID NO: 313, a LCDR3 as set forth in SEQ ID NO: 345, a HCDR1 as set forth in SEQ ID NO: 355, a HCDR2 as set forth in SEQ ID NO: 376 and a HCDR3 as set forth in SEQ ID NO: 420;
(11) a LCDR1 as set forth in SEQ ID NO: 262, a LCDR2 as set forth in SEQ ID NO: 292, a LCDR3 as set forth in SEQ ID NO: 320, a HCDR1 as set forth in SEQ ID NO: 350, a HCDR2 as set forth in SEQ ID NO: 371 and a HCDR3 as set forth in SEQ ID NO: 395;
(12) a LCDR1 as set forth in SEQ ID NO: 277, a LCDR2 as set forth in SEQ ID NO: 306, a LCDR3 as set forth in SEQ ID NO: 337, a HCDR1 as set forth in SEQ ID NO: 361, a HCDR2 as set forth in SEQ ID NO: 384 and a HCDR3 as set forth in SEQ ID NO: 411;
(13) a LCDR1 as set forth in SEQ ID NO: 287, a LCDR2 as set forth in SEQ ID NO: 314, a LCDR3 as set forth in SEQ ID NO: 346, a HCDR1 as set forth in SEQ ID NO: 365, a HCDR2 as set forth in SEQ ID NO: 391 and a HCDR3 as set forth in SEQ ID NO: 421;
(14) a LCDR1 as set forth in SEQ ID NO: 269, a LCDR2 as set forth in SEQ ID NO: 299, a LCDR3 as set forth in SEQ ID NO: 328, a HCDR1 as set forth in SEQ ID NO: 357, a HCDR2 as set forth in SEQ ID NO: 378 and a HCDR3 as set forth in SEQ ID NO: 402;
(15) a LCDR1 as set forth in SEQ ID NO: 270, a LCDR2 as set forth in SEQ ID NO: 300, a LCDR3 as set forth in SEQ ID NO: 329, a HCDR1 as set forth in SEQ ID NO: 358, a HCDR2 as set forth in SEQ ID NO: 379 and a HCDR3 as set forth in SEQ ID NO: 403;
(16) a LCDR1 as set forth in SEQ ID NO: 282, a LCDR2 as set forth in SEQ ID NO: 309, a LCDR3 as set forth in SEQ ID NO: 341, a HCDR1 as set forth in SEQ ID NO: 365, a HCDR2 as set forth in SEQ ID NO: 388 and a HCDR3 as set forth in SEQ ID NO: 416;
(17) a LCDR1 as set forth in SEQ ID NO: 265, a LCDR2 as set forth in SEQ ID NO: 295, a LCDR3 as set forth in SEQ ID NO: 323, a HCDR1 as set forth in SEQ ID NO: 352, a HCDR2 as set forth in SEQ ID NO: 373 and a HCDR3 as set forth in SEQ ID NO: 397;
(18) a LCDR1 as set forth in SEQ ID NO: 261, a LCDR2 as set forth in SEQ ID NO: 290, a LCDR3 as set forth in SEQ ID NO: 324, a HCDR1 as set forth in SEQ ID NO: 353, a HCDR2 as set forth in SEQ ID NO: 374 and a HCDR3 as set forth in SEQ ID NO: 398;
(19) a LCDR1 as set forth in SEQ ID NO: 271, a LCDR2 as set forth in SEQ ID NO: 301, a LCDR3 as set forth in SEQ ID NO: 330, a HCDR1 as set forth in SEQ ID NO: 351, a HCDR2 as set forth in SEQ ID NO: 380 and a HCDR3 as set forth in SEQ ID NO: 404;
(20) a LCDR1 as set forth in SEQ ID NO: 261, a LCDR2 as set forth in SEQ ID NO: 291, a LCDR3 as set forth in SEQ ID NO: 319, a HCDR1 as set forth in SEQ ID NO: 349, a HCDR2 as set forth in SEQ ID NO: 370 and a HCDR3 as set forth in SEQ ID NO: 394;
(21) a LCDR1 as set forth in SEQ ID NO: 279, a LCDR2 as set forth in SEQ ID NO: 307, a LCDR3 as set forth in SEQ ID NO: 338, a HCDR1 as set forth in SEQ ID NO: 362, a HCDR2 as set forth in SEQ ID NO: 385 and a HCDR3 as set forth in SEQ ID NO: 412;
(22) a LCDR1 as set forth in SEQ ID NO: 288, a LCDR2 as set forth in SEQ ID NO: 315, a LCDR3 as set forth in SEQ ID NO: 347, a HCDR1 as set forth in SEQ ID NO: 368, a HCDR2 as set forth in SEQ ID NO: 392 and a HCDR3 as set forth in SEQ ID NO: 422;
(23) a LCDR1 as set forth in SEQ ID NO: 272, a LCDR2 as set forth in SEQ ID NO: 299, a LCDR3 as set forth in SEQ ID NO: 331, a HCDR1 as set forth in SEQ ID NO: 356, a HCDR2 as set forth in SEQ ID NO: 377 and a HCDR3 as set forth in SEQ ID NO: 405;
(24) a LCDR1 as set forth in SEQ ID NO: 273, a LCDR2 as set forth in SEQ ID NO: 302, a LCDR3 as set forth in SEQ ID NO: 332, a HCDR1 as set forth in SEQ ID NO: 355, a HCDR2 as set forth in SEQ ID NO: 376 and a HCDR3 as set forth in SEQ ID NO: 406;
(25) a LCDR1 as set forth in SEQ ID NO: 261, a LCDR2 as set forth in SEQ ID NO: 290, a LCDR3 as set forth in SEQ ID NO: 317, a HCDR1 as set forth in SEQ ID NO: 349, a HCDR2 as set forth in SEQ ID NO: 370 and a HCDR3 as set forth in SEQ ID NO: 394;
(26) a LCDR1 as set forth in SEQ ID NO: 280, a LCDR2 as set forth in SEQ ID NO: 308, a LCDR3 as set forth in SEQ ID NO: 339, a HCDR1 as set forth in SEQ ID NO: 363, a HCDR2 as set forth in SEQ ID NO: 386 and a HCDR3 as set forth in SEQ ID NO: 413;
(27) a LCDR1 as set forth in SEQ ID NO: 274, a LCDR2 as set forth in SEQ ID NO: 303, a LCDR3 as set forth in SEQ ID NO: 327, a HCDR1 as set forth in SEQ ID NO: 356, a HCDR2 as set forth in SEQ ID NO: 377 and a HCDR3 as set forth in SEQ ID NO: 401;
(28) a LCDR1 as set forth in SEQ ID NO: 261, a LCDR2 as set forth in SEQ ID NO: 290, a LCDR3 as set forth in SEQ ID NO: 318, a HCDR1 as set forth in SEQ ID NO: 349, a HCDR2 as set forth in SEQ ID NO: 370 and a HCDR3 as set forth in SEQ ID NO: 394;
(29) a LCDR1 as set forth in SEQ ID NO: 262, a LCDR2 as set forth in SEQ ID NO: 292, a LCDR3 as set forth in SEQ ID NO: 320, a HCDR1 as set forth in SEQ ID NO: 350, a HCDR2 as set forth in SEQ ID NO: 371 and a HCDR3 as set forth in SEQ ID NO: 395;
(30) a LCDR1 as set forth in SEQ ID NO: 275, a LCDR2 as set forth in SEQ ID NO: 304, a LCDR3 as set forth in SEQ ID NO: 333, a HCDR1 as set forth in SEQ ID NO: 359, a HCDR2 as set forth in SEQ ID NO: 381 and a HCDR3 as set forth in SEQ ID NO: 407;
(31) a LCDR1 as set forth in SEQ ID NO: 289, a LCDR2 as set forth in SEQ ID NO: 316, a LCDR3 as set forth in SEQ ID NO: 348, a HCDR1 as set forth in SEQ ID NO: 369, a HCDR2 as set forth in SEQ ID NO: 393 and a HCDR3 as set forth in SEQ ID NO: 423;
(32) a LCDR1 as set forth in SEQ ID NO: 282, a LCDR2 as set forth in SEQ ID NO: 309, a LCDR3 as set forth in SEQ ID NO: 341, a HCDR1 as set forth in SEQ ID NO: 364, a HCDR2 as set forth in SEQ ID NO: 387 and a HCDR3 as set forth in SEQ ID NO: 415;
(33) a LCDR1 as set forth in SEQ ID NO: 276, a LCDR2 as set forth in SEQ ID NO: 302, a LCDR3 as set forth in SEQ ID NO: 334, a HCDR1 as set forth in SEQ ID NO: 355, a HCDR2 as set forth in SEQ ID NO: 376 and a HCDR3 as set forth in SEQ ID NO: 408;
(34) a LCDR1 as set forth in SEQ ID NO: 266, a LCDR2 as set forth in SEQ ID NO: 296, a LCDR3 as set forth in SEQ ID NO: 325, a HCDR1 as set forth in SEQ ID NO: 354, a HCDR2 as set forth in SEQ ID NO: 375 and a HCDR3 as set forth in SEQ ID NO: 399; and
(35) a LCDR1 as set forth in SEQ ID NO: 281, a LCDR2 as set forth in SEQ ID NO: 305, a LCDR3 as set forth in SEQ ID NO: 340, a HCDR1 as set forth in SEQ ID NO: 349, a HCDR2 as set forth in SEQ ID NO: 382 and a HCDR3 as set forth in SEQ ID NO: 414.

In some embodiments, the anti-CD123 antibody comprises a light chain variable region and a heavy chain variable region selected from the following group:
(1) a light chain variable region as set forth in SEQ ID NO: 424 and a heavy chain variable region as set forth in SEQ ID NO: 459;
(2) a light chain variable region as set forth in SEQ ID NO: 425 and a heavy chain variable region as set forth in SEQ ID NO: 460;
(3) a light chain variable region as set forth in SEQ ID NO: 426 and a heavy chain variable region as set forth in SEQ ID NO: 461;
(4) a light chain variable region as set forth in SEQ ID NO: 427 and a heavy chain variable region as set forth in SEQ ID NO: 462;
(5) a light chain variable region as set forth in SEQ ID NO: 428 and a heavy chain variable region as set forth in SEQ ID NO: 463;
(6) a light chain variable region as set forth in SEQ ID NO: 429 and a heavy chain variable region as set forth in SEQ ID NO: 464;
(7) a light chain variable region as set forth in SEQ ID NO: 430 and a heavy chain variable region as set forth in SEQ ID NO: 465;
(8) a light chain variable region as set forth in SEQ ID NO: 431 and a heavy chain variable region as set forth in SEQ ID NO: 466;
(9) a light chain variable region as set forth in SEQ ID NO: 432 and a heavy chain variable region as set forth in SEQ ID NO: 467;
(10) a light chain variable region as set forth in SEQ ID NO: 433 and a heavy chain variable region as set forth in SEQ ID NO: 468;
(11) a light chain variable region as set forth in SEQ ID NO: 434 and a heavy chain variable region as set forth in SEQ ID NO: 469;
(12) a light chain variable region as set forth in SEQ ID NO: 435 and a heavy chain variable region as set forth in SEQ ID NO: 470;
(13) a light chain variable region as set forth in SEQ ID NO: 436 and a heavy chain variable region as set forth in SEQ ID NO: 471;
(14) a light chain variable region as set forth in SEQ ID NO: 437 and a heavy chain variable region as set forth in SEQ ID NO: 472;
(15) a light chain variable region as set forth in SEQ ID NO: 438 and a heavy chain variable region as set forth in SEQ ID NO: 473;
(16) a light chain variable region as set forth in SEQ ID NO: 439 and a heavy chain variable region as set forth in SEQ ID NO: 474;
(17) a light chain variable region as set forth in SEQ ID NO: 440 and a heavy chain variable region as set forth in SEQ ID NO: 475;
(18) a light chain variable region as set forth in SEQ ID NO: 441 and a heavy chain variable region as set forth in SEQ ID NO: 476;
(19) a light chain variable region as set forth in SEQ ID NO: 442 and a heavy chain variable region as set forth in SEQ ID NO: 477;
(20) a light chain variable region as set forth in SEQ ID NO: 443 and a heavy chain variable region as set forth in SEQ ID NO: 478;
(21) a light chain variable region as set forth in SEQ ID NO: 444 and a heavy chain variable region as set forth in SEQ ID NO: 479;
(22) a light chain variable region as set forth in SEQ ID NO: 445 and a heavy chain variable region as set forth in SEQ ID NO: 480;
(23) a light chain variable region as set forth in SEQ ID NO: 446 and a heavy chain variable region as set forth in SEQ ID NO: 481;
(24) a light chain variable region as set forth in SEQ ID NO: 447 and a heavy chain variable region as set forth in SEQ ID NO: 482;
(25) a light chain variable region as set forth in SEQ ID NO: 448 and a heavy chain variable region as set forth in SEQ ID NO: 483;
(26) a light chain variable region as set forth in SEQ ID NO: 449 and a heavy chain variable region as set forth in SEQ ID NO: 484;
(27) a light chain variable region as set forth in SEQ ID NO: 450 and a heavy chain variable region as set forth in SEQ ID NO: 485;
(28) a light chain variable region as set forth in SEQ ID NO: 451 and a heavy chain variable region as set forth in SEQ ID NO: 486;
(29) a light chain variable region as set forth in SEQ ID NO: 452 and a heavy chain variable region as set forth in SEQ ID NO: 487;
(30) a light chain variable region as set forth in SEQ ID NO: 453 and a heavy chain variable region as set forth in SEQ ID NO: 488;
(31) a light chain variable region as set forth in SEQ ID NO: 454 and a heavy chain variable region as set forth in SEQ ID NO: 489;
(32) a light chain variable region as set forth in SEQ ID NO: 455 and a heavy chain variable region as set forth in SEQ ID NO: 490;
(33) a light chain variable region as set forth in SEQ ID NO: 456 and a heavy chain variable region as set forth in SEQ ID NO: 491;
(34) a light chain variable region as set forth in SEQ ID NO: 457 and a heavy chain variable region as set forth in SEQ ID NO: 492; and
(35) a light chain variable region as set forth in SEQ ID NO: 458 and a heavy chain variable region as set forth in SEQ ID NO: 493.

In some embodiments, the anti-CD123 antibody is an anti-CD123 scFv, which may comprise an amino acid sequence selected from SEQ ID NOs: 497-531.

In some embodiments, the antibody specifically binds to BCMA, which is also called anti-BCMA antibody, is the antibody described in PCT/CN2022/112728 (which is incorporated herein by reference in its entirety), including a light chain variable region comprising LCDR1, LCDR2 and LCDR3 and a heavy chain variable region comprising HCDR1, HCDR2, and HCDR3, wherein LCDR1, LCDR2, LCDR3, HCDR1, HCDR2, and HCDR3 are selected from the following group:
(1) a LCDR1 as set forth in SEQ ID NO: 146, a LCDR2 as set forth in SEQ ID NO: 156, a LCDR3 as set forth in SEQ ID NO: 167, a HCDR1 as set forth in SEQ ID NO: 178, a HCDR2 as set forth in SEQ ID NO: 189 and a HCDR3 as set forth in SEQ ID NO: 201;
(2) a LCDR1 as set forth in SEQ ID NO: 147, a LCDR2 as set forth in SEQ ID NO: 157, a LCDR3 as set forth in SEQ ID NO: 168, a HCDR1 as set forth in SEQ ID NO: 179, a HCDR2 as set forth in SEQ ID NO: 190 and a HCDR3 as set forth in SEQ ID NO: 202;
(3) a LCDR1 as set forth in SEQ ID NO: 148, a LCDR2 as set forth in SEQ ID NO: 158, a LCDR3 as set forth in SEQ ID NO: 169, a HCDR1 as set forth in SEQ ID NO: 180, a HCDR2 as set forth in SEQ ID NO: 191 and a HCDR3 as set forth in SEQ ID NO: 203;
(4) a LCDR1 as set forth in SEQ ID NO: 149, a LCDR2 as set forth in SEQ ID NO: 159, a LCDR3 as set forth in SEQ ID NO: 169, a HCDR1 as set forth in SEQ ID NO: 181, a HCDR2 as set forth in SEQ ID NO: 192 and a HCDR3 as set forth in SEQ ID NO: 204;
(5) a LCDR1 as set forth in SEQ ID NO: 150, a LCDR2 as set forth in SEQ ID NO: 160, a LCDR3 as set forth in SEQ ID NO: 170, a HCDR1 as set forth in SEQ ID NO: 182, a HCDR2 as set forth in SEQ ID NO: 193 and a HCDR3 as set forth in SEQ ID NO: 205;
(6) a LCDR1 as set forth in SEQ ID NO: 151, a LCDR2 as set forth in SEQ ID NO: 161, a LCDR3 as set forth in SEQ ID NO: 171, a HCDR1 as set forth in SEQ ID NO: 183, a HCDR2 as set forth in SEQ ID NO: 194 and a HCDR3 as set forth in SEQ ID NO: 206;
(7) a LCDR1 as set forth in SEQ ID NO: 152, a LCDR2 as set forth in SEQ ID NO: 162, a LCDR3 as set forth in SEQ ID NO: 172, a HCDR1 as set forth in SEQ ID NO: 180, a HCDR2 as set forth in SEQ ID NO: 195 and a HCDR3 as set forth in SEQ ID NO: 207;
(8) a LCDR1 as set forth in SEQ ID NO: 153, a LCDR2 as set forth in SEQ ID NO: 163, a LCDR3 as set forth in SEQ ID NO: 173, a HCDR1 as set forth in SEQ ID NO: 184, a HCDR2 as set forth in SEQ ID NO: 196 and a HCDR3 as set forth in SEQ ID NO: 208;
(9) a LCDR1 as set forth in SEQ ID NO: 147, a LCDR2 as set forth in SEQ ID NO: 164, a LCDR3 as set forth in SEQ ID NO: 174, a HCDR1 as set forth in SEQ ID NO: 185, a HCDR2 as set forth in SEQ ID NO: 197 and a HCDR3 as set forth in SEQ ID NO: 209;
(10) a LCDR1 as set forth in SEQ ID NO: 147, a LCDR2 as set forth in SEQ ID NO: 165, a LCDR3 as set forth in SEQ ID NO: 175, a HCDR1 as set forth in SEQ ID NO: 186, a HCDR2 as set forth in SEQ ID NO: 198 and a HCDR3 as set forth in SEQ ID NO: 210;
(11) a LCDR1 as set forth in SEQ ID NO: 154, a LCDR2 as set forth in SEQ ID NO: 166, a LCDR3 as set forth in SEQ ID NO: 176, a HCDR1 as set forth in SEQ ID NO: 187, a HCDR2 as set forth in SEQ ID NO: 199 and a HCDR3 as set forth in SEQ ID NO: 211; and
(12) a LCDR1 as set forth in SEQ ID NO: 155, a LCDR2 as set forth in SEQ ID NO: 159, a LCDR3 as set forth in SEQ ID NO: 177, a HCDR1 as set forth in SEQ ID NO: 188, a HCDR2 as set forth in SEQ ID NO: 200 and a HCDR3 as set forth in SEQ ID NO: 212.

In some embodiments, the anti-BCMA antibody comprises a light chain variable region and a heavy chain variable region selected from the following group:
(1) a light chain variable region as set forth in SEQ ID NO: 213 and a heavy chain variable region as set forth in SEQ ID NO: 225;
(2) a light chain variable region as set forth in SEQ ID NO: 214 and a heavy chain variable region as set forth in SEQ ID NO: 226;
(3) a light chain variable region as set forth in SEQ ID NO: 215 and a heavy chain variable region as set forth in SEQ ID NO: 227;
(4) a light chain variable region as set forth in SEQ ID NO: 216 and a heavy chain variable region as set forth in SEQ ID NO: 228;
(5) a light chain variable region as set forth in SEQ ID NO: 217 and a heavy chain variable region as set forth in SEQ ID NO: 229;
(6) a light chain variable region as set forth in SEQ ID NO: 218 and a heavy chain variable region as set forth in SEQ ID NO: 230;
(7) a light chain variable region as set forth in SEQ ID NO: 219 and a heavy chain variable region as set forth in SEQ ID NO: 231;
(8) a light chain variable region as set forth in SEQ ID NO: 220 and a heavy chain variable region as set forth in SEQ ID NO: 232;
(9) a light chain variable region as set forth in SEQ ID NO: 221 and a heavy chain variable region as set forth in SEQ ID NO: 233;
(10) a light chain variable region as set forth in SEQ ID NO: 222 and a heavy chain variable region as set forth in SEQ ID NO: 234;
(11) a light chain variable region as set forth in SEQ ID NO: 223 and a heavy chain variable region as set forth in SEQ ID NO: 235; and
(12) a light chain variable region as set forth in SEQ ID NO: 224 and a heavy chain variable region as set forth in SEQ ID NO: 236.

In some embodiments, the anti-BCMA antibody is an anti-BCMA scFv, which may comprise an amino acid sequence selected from SEQ ID NOs: 237-248.

In some embodiments, the antibody specifically binds to CD19, which is also called anti-CD19 antibody, is the antibody described in CN202210274255.1 (published as CN114349863A, which is incorporated herein by reference in its entirety), including a light chain variable region comprising LCDR1, LCDR2 and LCDR3 and a heavy chain variable region comprising HCDR1, HCDR2, and HCDR3, wherein LCDR1, LCDR2, LCDR3, HCDR1, HCDR2, and HCDR3 are: a LCDR1 as set forth in SEQ ID NO: 542, a LCDR2 as set forth in SEQ ID NO: 543, a LCDR3 as set forth in SEQ ID NO: 544, a HCDR1 as set forth in SEQ ID NO: 545, a HCDR2 as set forth in SEQ ID NO: 546 and a HCDR3 as set forth in SEQ ID NO: 547.

In some embodiments, the anti-CD19 antibody comprises a light chain variable region as set forth in SEQ ID NO: 549 and a heavy chain variable region as set forth in SEQ ID NO: 548. In some embodiments, the anti-CD19 antibody is an anti-CD19 scFv, which may comprise an amino acid sequence as set forth in SEQ ID NO: 550.

In some embodiments, the antibody specifically binds to HER2, which is also called anti-HER2 antibody, is the antibody described in CN202210750853.1 (published as CN114805584A, which is incorporated herein by reference in its entirety), including a light chain variable region comprising LCDR1, LCDR2 and LCDR3 and a heavy chain variable region comprising HCDR1, HCDR2, and HCDR3, wherein LCDR1, LCDR2, LCDR3, HCDR1, HCDR2, and HCDR3 are: a LCDR1 as set forth in SEQ ID NO: 532, a LCDR2 as set forth in SEQ ID NO: 533, a LCDR3 as set forth in SEQ ID NO: 534, a HCDR1 as set forth in SEQ ID NO: 535, a HCDR2 as set forth in SEQ ID NO: 536 and a HCDR3 as set forth in SEQ ID NO: 537.

In some embodiments, the anti-HER2 antibody comprises a light chain variable region as set forth in SEQ ID NO: 538 and a heavy chain variable region as set forth in SEQ ID NO: 539. In some embodiments, the anti-HER2 antibody is an anti-HER2 scFv, which may comprise an amino acid sequence as set forth in SEQ ID NO: 540.

In some embodiments, the antibody specifically binds to IL13Ra2, which is also called anti-IL13Ra2 antibody, is the antibody described in CN202210743595.4 (published as CN114805581A, which is incorporated herein by reference in its entirety) including a light chain variable region comprising LCDR1, LCDR2 and LCDR3 and a heavy chain variable region comprising HCDR1, HCDR2, and HCDR3, wherein LCDR1, LCDR2, LCDR3, HCDR1, HCDR2, and HCDR3 are: a LCDR1 as set forth in SEQ ID NO: 552, a LCDR2 as set forth in SEQ ID NO: 553, a LCDR3 as set forth in SEQ ID NO: 554, a HCDR1 as set forth in SEQ ID NO: 555, a HCDR2 as set forth in SEQ ID NO: 556 and a HCDR3 as set forth in SEQ ID NO: 557.

In some embodiments, the anti-IL13Ra2 antibody comprises a light chain variable region as set forth in SEQ ID NO: 558 and a heavy chain variable region as set forth in SEQ ID NO: 559. In some embodiments, the anti-IL13Ra2 antibody is an anti-IL13Ra2 scFv, which may comprise an amino acid sequence as set forth in SEQ ID NO: 560.

In some embodiments, the antibody specifically binds to B7H3, which is also called anti-B7H3 antibody, is the antibody described in CN202210714289.8 (published as CN114773477A, which is incorporated herein by reference in its entirety), including a light chain variable region comprising LCDR1, LCDR2 and LCDR3 and a heavy chain variable region comprising HCDR1, HCDR2, and HCDR3, wherein LCDR1, LCDR2, LCDR3, HCDR1, HCDR2, and HCDR3 are: a LCDR1 as set forth in SEQ ID NO: 562, a LCDR2 as set forth in SEQ ID NO: 563, a LCDR3 as set forth in SEQ ID NO: 564, a HCDR1 as set forth in SEQ ID NO: 565, a HCDR2 as set forth in SEQ ID NO: 566 and a HCDR3 as set forth in SEQ ID NO: 567.

In some embodiments, the anti-B7H3 antibody comprises a light chain variable region as set forth in SEQ ID NO: 568 and a heavy chain variable region as set forth in SEQ ID NO: 569. In some embodiments, the anti-B7H3 antibody is an anti-B7H3 scFv, which may comprise an amino acid sequence as set forth in SEQ ID NO: 570.

In some embodiments, the antibody specifically binds to CD40, which is also called anti-CD40 antibody, is the antibody described in PCT/CN2022/112730 (which is incorporated herein by reference in its entirety), including a light chain variable region comprising LCDR1, LCDR2 and LCDR3 and a heavy chain variable region comprising HCDR1, HCDR2, and HCDR3, wherein LCDR1, LCDR2, LCDR3, HCDR1, HCDR2, and HCDR3 are selected from the following group:
(1) a LCDR1 as set forth in SEQ ID NO: 828, a LCDR2 as set forth in SEQ ID NO: 834, a LCDR3 as set forth in SEQ ID NO: 840, a HCDR1 as set forth in SEQ ID NO: 846, a HCDR2 as set forth in SEQ ID NO: 852 and a HCDR3 as set forth in SEQ ID NO: 858;
(2) a LCDR1 as set forth in SEQ ID NO: 829, a LCDR2 as set forth in SEQ ID NO: 835, a LCDR3 as set forth in SEQ ID NO: 841, a HCDR1 as set forth in SEQ ID NO: 847, a HCDR2 as set forth in SEQ ID NO: 853 and a HCDR3 as set forth in SEQ ID NO: 859;
(3) a LCDR1 as set forth in SEQ ID NO: 830, a LCDR2 as set forth in SEQ ID NO: 836, a LCDR3 as set forth in SEQ ID NO: 842, a HCDR1 as set forth in SEQ ID NO: 848, a HCDR2 as set forth in SEQ ID NO: 854 and a HCDR3 as set forth in SEQ ID NO: 860;
(4) a LCDR1 as set forth in SEQ ID NO: 831, a LCDR2 as set forth in SEQ ID NO: 837, a LCDR3 as set forth in SEQ ID NO: 843, a HCDR1 as set forth in SEQ ID NO: 849, a HCDR2 as set forth in SEQ ID NO: 855 and a HCDR3 as set forth in SEQ ID NO: 861;
(5) a LCDR1 as set forth in SEQ ID NO: 832, a LCDR2 as set forth in SEQ ID NO: 838, a LCDR3 as set forth in SEQ ID NO: 844, a HCDR1 as set forth in SEQ ID NO: 850, a HCDR2 as set forth in SEQ ID NO: 856 and a HCDR3 as set forth in SEQ ID NO: 862; and
(6) a LCDR1 as set forth in SEQ ID NO: 833, a LCDR2 as set forth in SEQ ID NO: 839, a LCDR3 as set forth in SEQ ID NO: 845, a HCDR1 as set forth in SEQ ID NO: 851, a HCDR2 as set forth in SEQ ID NO: 857 and a HCDR3 as set forth in SEQ ID NO: 863.

In some embodiments, the anti-CD40 antibody comprises a light chain variable region and a heavy chain variable region selected from the following group:
(1) a light chain variable region as set forth in SEQ ID NO: 864 and a heavy chain variable region as set forth in SEQ ID NO: 870;
(2) a light chain variable region as set forth in SEQ ID NO: 865 and a heavy chain variable region as set forth in SEQ ID NO: 971;
(3) a light chain variable region as set forth in SEQ ID NO: 866 and a heavy chain variable region as set forth in SEQ ID NO: 872;
(4) a light chain variable region as set forth in SEQ ID NO: 867 and a heavy chain variable region as set forth in SEQ ID NO: 873;
(5) a light chain variable region as set forth in SEQ ID NO: 868 and a heavy chain variable region as set forth in SEQ ID NO: 874; and
(6) a light chain variable region as set forth in SEQ ID NO: 869 and a heavy chain variable region as set forth in SEQ ID NO: 875.

In some embodiments, the anti-CD40 antibody is an anti-CD40 scFv, which may comprise an amino acid sequence selected from SEQ ID NOs: 888-893.

In some embodiments, the protein coding sequence may encode a CAR. In some embodiments, the CAR comprises a binding domain which can specifically bind to mesothelin, CD123, BCMA, HER2, IL13Ra2, B7H3 or CD40. In some embodiments, the binding domain may comprise an antibody that specifically binds to mesothelin, CD123, BCMA, HER2, IL13Ra2, B7H3 or CD40. In some embodiments, the binding domain may be a scFv that specifically binds to mesothelin, CD123, BCMA, HER2, IL13Ra2, B7H3 or CD40.

The CAR may also comprise a signaling peptide, a hinge region, a transmembrane domain and an intracellular signaling domain. The intracellular signaling domain may further comprise a co-stimulatory domain. In some embodiments, the signaling peptide may comprise a CD8 signal peptide or a GM-CSF signal peptide. In some embodiments, the hinge region of the CAR may comprise a hinge domain of CD28, CD8, IgG1, IgG4, IgD, 4-1B1. (D4, CD27, CD7, CD8A, PD-1, ICOS, OX40, NKG2D, NKG2C, FcεRIγ, BTLA, GIIR, DAP10, TIM1, SLAM, CD30 or LIGHT, preferably, a CD8 hinge domain. In some embodiments, the transmembrane domain of the CAR may comprise the transmembrane domain of CD8, CD28, CD3ε (CD3e), 4-1BB, CD4, CD27, CD7, PD-1, TRAC, TRBC, CD3ζ, CTLA-4, LAG-3, CD5, ICOS, OX40, NKG2D, 2B4, CD244, FcεRIγ, BTLA, CD30, GITR, HVEM, DAP10, CD2, NKG2C, LIGHT, DAP12. CD40L (CD154), TIM1, CD226, DR3, CD45, CD80, CD86, CD9, CD16, CD22, CD33, CD37, CD64 or SLAM, preferably a CD8 transmembrane (TM) domain. In some embodiments, the intracellular signaling domain of the CAR may comprise the intracellular signaling domain of CD3ζ, CD3δ, CD3γ, CD3ε, CD79a, CD79b, FeeRIγ, FceRIβ, FcγRlla, DA P10 or DAP-12, preferably CD3ζ intracellular signaling domain. In some embodiments, the intracellular signaling domain of the CAR may further comprise a co-stimulatory domain, such as the co-stimulatory domain of CD28, 4-1BB (CD137), CD27, CD2, CD7, CD8A, CD8B, OX40, CD226, DR3, SLAM, CDS, ICAM-1, NKG2D, NKG2C, B7413, 2B4, FcεRIγ, BTLA, GITR, HVEM, DAP10, DAP12, CD30, CD40, CD40L, TIM1, PD-1, LFA-1, LIGHT, JAML, CD244, CD100, ICOS, CD40 or MyD88, preferably a 4-1BB co-stimulatory domain.

In some embodiments, a CAR with “BBZ” refers to a CAR with 4-1BB co-stimulatory molecules, typically comprising a CD8 hinge domain, a CD8 transmembrane (TM) domain, a 4-1BB costimulatory domain and CD3ζ domain.

In some embodiments, the CAR comprises a binding domain which can specifically bind to mesothelin (e.g., an antibody against mesothelin, such as an anti-mesothelin scFv), which can be called a CAR targeting mesothelin. In some embodiments, the CAR targeting mesothelin may be the CAR described in PCT/CN2022/112726 (which is incorporated herein by reference in its entirety). In some embodiments, the CAR targeting mesothelin may comprise any one of the anti-mesothelin antibodies as described above. In some embodiments, the CAR targeting mesothelin may comprise an amino acid sequence selected from SEQ ID Nos: 131-145.

In some embodiments, the CAR comprises a binding domain which can specifically bind to CD123 (e.g., an antibody against CD123, such as an anti-CD123 scFv), which can be called a CAR targeting CD123. In some embodiments, the CAR targeting CD123 may be the CAR described in PCT/CN2022/112724 (which is incorporated herein by reference in its entirety). In some embodiments, the CAR targeting CD123 may comprise any one of the anti-CD123 antibodies as described above. In some embodiments, the CAR targeting CD123 may comprise an amino acid sequence selected from SEQ ID Nos: 494-496.

In some embodiments, the CAR comprises a binding domain which can specifically bind to BCMA (e.g., an antibody against BCMA, such as an anti-BCM scFv), which can be called a CAR targeting BCMA. In some embodiments, the CAR targeting BCMA may be the CAR described in PCT/CN2022/112728 (which is incorporated herein by reference in its entirety). In some embodiments, the CAR targeting BCMA may comprise any one of the anti-BCMA antibodies as described above. In some embodiments, the CAR targeting BCMA may comprise an amino acid sequence selected from SEQ ID Nos: 249-260.

In some embodiments, the CAR comprises a binding domain which can specifically bind to CD19 (e.g., an antibody against CD19, such as an anti-CD19 scFv), which can be called a CAR targeting CD19. In some embodiments, the CAR targeting CD19 may be the CAR described in CN202210274255.1 (published as CN114349863A). In some embodiments, the CAR targeting CD19 may comprise any one of the anti-CD19 antibodies as described above. In some embodiments, the CAR targeting CD19 may comprise an amino acid sequence as set forth in SEQ ID No: 551.

In some embodiments, the CAR comprises a binding domain which can specifically bind to HER2 (e.g., an antibody against HER2, such as an anti-HER2 scFv), which can be called a CAR targeting HER2. In some embodiments, the CAR targeting HER2 may be the CAR described in CN202210750853.1 (published as CN114805584A, which is incorporated herein by reference in its entirety). In some embodiments, the CAR targeting HER2 may comprise any one of the anti-HER2 antibodies as described above. In some embodiments, the CAR targeting HER2 may comprise an amino acid sequence as set forth in SEQ ID No: 541.

In some embodiments, the CAR comprises a binding domain which can specifically bind to IL13Ra2 (e.g., an antibody against IL13Ra2, such as an anti-IL13Ra2 scFv), which can be called a CAR targeting IL13Ra2. In some embodiments, the CAR targeting IL13Ra2 may be the CAR described in CN202210743595.4 (published as CN114805581A, which is incorporated herein by reference in its entirety). In some embodiments, the CAR targeting IL13Ra2 may comprise any one of the anti-IL13Ra2 antibodies as described above. In some embodiments, the CAR targeting IL13Ra2 may comprise an amino acid sequence as set forth in SEQ ID No: 561.

In some embodiments, the CAR comprises a binding domain which can specifically bind to B7H3 (e.g., an antibody against B7H3, such as an anti B7H3 scFv), which can be called a CAR targeting B7H3. In some embodiments, the CAR targeting B7H3 may be the CAR described in CN202210714289.8 (published as CN114773477A, which is incorporated herein by reference in its entirety). In some embodiments, the CAR targeting B7H3 may comprise any one of the anti-B7H3 antibodies as described above. In some embodiments, the CAR targeting B7H3 may comprise an amino acid sequence as set forth in SEQ ID No: 571.

In some embodiments, the protein coding sequence may encode a fusion protein that are referred to as Lymphocytes-Antigen presenting cells Co-stimulators (“LACOSTIMs”, which is also called LACO or LACO-Stim herein), e.g., as described in PCT/CN2021/112742 and PCT/CN2022/112730 (which are incorporated herein by reference in their entirety). Fusion proteins provided herein comprise a first domain that activates an antigen-presenting cell (APC) and a second domain that activates an immune effector cell, wherein (i) the first domain comprises (a) a ligand that binds to an activation receptor of the APC, or a receptor-binding fragment thereof, or (b) an antibody that binds to an activation receptor of the APC, or an antigen-binding fragment thereof; and (ii) the second domain comprises (a) a co-stimulatory receptor of the immune effector cell, or a functional fragment thereof, (b) a co-stimulatory ligand of the immune effector cell, or a receptor-binding fragment thereof, or (c) an antibody that binds to a co-stimulatory receptor of the immune effector cell, or an antigen-binding fragment thereof.

In some embodiments, the APC is selected from the group consisting of a dendritic cell, a macrophage, a myeloid derived suppressor cell, a monocyte, a B cell, a T cell, and a Langerhans cell. In some embodiments, the activation receptor of the APC is selected from the group consisting of CD40, CD80, CD86, CD91, DEC-205 and DC-SIGN.

In some embodiments, the first domain of the fusion proteins provided herein comprises an antibody that binds to the activation receptor of the APC, or an antigen-binding fragment thereof. In some embodiments, the first domain of the fusion proteins provided herein is an anti-CD40 antibody or an antigen-binding fragment thereof. In some embodiments, the first domain is a monoclonal antibody. In some embodiments, the first domain is a chimeric, humanized, or human antibody. In some embodiments, the first domain is a Fab, Fab′, F(ab′)₂, Fv, scFv, (scFv)₂, single chain antibody, dual variable region antibody, diabody, nanobody, or single variable region antibody.

In some embodiments, the first domain of the fusion proteins provided herein is an anti-CD40 antibody or an antigen-binding fragment thereof. In some embodiments, the first domain of the fusion proteins provided herein is an anti-CD40 scFv. In some embodiments, the anti-CD40 antibody is any one anti-CD40 antibody listed herein. In some embodiments, the fusion protein comprises a first domain that activates an antigen-presenting cell (APC) and a second domain that activates an immune effector cell, wherein the immune effector cell is selected from the group consisting of a T cell, an NK cell, an NKT cell, a macrophage, a neutrophil, and a granulocyte. In some embodiments, the second domain of the fusion proteins provided herein comprises a cytoplasmic domain of the co-stimulatory receptor. In some embodiments, the co-stimulatory receptor is selected from the group consisting of CD28, 4-1BB, ICOS, CD27. OX40, DAP10, 2134, CD30, CD2, LIGHT, GITR, TLR, DR3, and CD43. In some embodiments, the co-stimulatory receptor is CD28. In some embodiments, the co-stimulatory receptor is 4-1BB. In some embodiments, the second domain further comprises the transmembrane domain of the co-stimulatory receptor.

In some embodiments, the second domain of the fusion proteins provided herein is a co-stimulatory ligand of the immune effector cell, or a receptor-binding fragment thereof. In some embodiments, the co-stimulatory ligand is selected from the group consisting of CD58, CD70, CD83, CD80, CD86, CD137L, CD252, CD275, CD54, CD49a, CD112, CD150, CD155, CD265, CD270, TL1A, CD127, IL-4R, GITR-L, TIM-4, CD153, CD48, CD160. CD200R, and CD44.

In some embodiments, the second domain of the fusion proteins provided herein is an antibody that binds to the co-stimulatory receptor, or an antigen-binding fragment thereof. In some embodiments, the co-stimulatory receptor is selected from the group consisting of CD28, 4-1BB, ICOS, CD27, OX40, DAP10, 2B4, CD30, CD2, LIGHT, GITR, TLR, DR3, and CD43. In some embodiments, the co-stimulatory receptor is CD28. In some embodiments, the co-stimulatory receptor is 4-1BB. In some embodiments, the second domain is a monoclonal antibody. In some embodiments, the second domain is a chimeric, humanized, or human antibody. In some embodiments, the second domain is a Fab, Fab′, F(ab′)₂, Fv, scFv, (scFv)₂, single chain antibody, dual variable region antibody, diabody, nanobody, or single variable region antibody.

In some embodiments, the second domain of the fusion proteins provided herein is an antibody that binds to (CD28, or an antigen-binding fragment thereof. In some embodiments.

In some embodiments, the fusion proteins provided herein, the N-terminus of the first domain is linked to the C-terminus of the second domain. In some embodiments, the N-terminus of the second domain is linked to the C-terminus of the first domain. In some embodiments, the first domain and the second domain of the fusion proteins provided herein are linked via a linker. In some embodiments, the linker is a trimerization motif. In some embodiments, the linker is a T4 fibritin trimerization motif.

In some embodiments of the fusion proteins provided herein, the first domain comprises CD40L or a receptor-binding fragment thereof, and the second domain comprises a CD28 cytoplasmic domain. In some embodiments, the first domain comprises a CD40L. In Some embodiments, the N-terminus of the first domain is linked to the C-terminus of the second domain.

In some embodiments of the fusion proteins provided herein, the first domain comprises CD40L or a receptor-binding fragment thereof, and the second domain comprises an anti-CD28 antibody or an antigen-binding fragment thereof. In some embodiments, the N-terminus of the first domain is linked to the C-terminus of the second domain. In some embodiments, the two domains are linked via a T4 fibritin trimerization motif.

In some embodiments of the fusion proteins provided herein, the first domain comprises an anti-CD40 antibody or an antigen-binding fragment thereof, and the second domain comprises an anti-CD28 antibody or an antigen-binding fragment thereof. In some embodiments, the N-terminus of the first domain is linked to the C-terminus of the second domain.

In some embodiments of the fusion proteins provided herein, the first domain comprises an anti-CD40 antibody or an antigen-binding fragment thereof, and the second domain comprises a CD28 transmembrane region and a CD28 cytoplasmic domain. In some embodiments, the first and second domains are linked via a CD8 hinge, a CD28 hinge, or an IgG Fc region. In some embodiments, the N-terminus of the second domain is linked to the C-terminus of the first domain.

In some embodiments, the LACOSTIM comprise a sequence selected from SEQ ID NO: 600, SEQ ID NO: 695-708, 801, 803, 813, 894-899.

In some embodiments, the protein coding sequence may encode a first protein and a second protein. The first protein may be an antibody, a chimeric antigen receptor (CAR) or a T cell receptor (TCR), and the second protein may be a LACOSTIM molecule described herein. The first protein and the second protein may be fused together as a fusion protein. The first protein may be linked to the N-terminus or the C-terminus of the second protein. The first protein and the second protein can be linked by a linker. The linker can be a self-cleaving linker, such as a 2A peptide (e.g., P2A, F2A, T2A etc.).

The term “polyA”, as used herein, is an abbreviation of polyadenylation and refers to a sequence comprising consecutive adenine nucleotides with a length of at least 30. The polyA sequence may be a ribonucleic acid sequence or a deoxyribonucleic acid sequence. The length of the consecutive adenine nucleotides in the polyA sequence may be at least 30 nucleotides, e.g., at least 45 nucleotides, at least 50 nucleotides, at least 55 nucleotides, at least 60 nucleotides, at least 65 nucleotides, at least 70 nucleotides, at least 75 nucleotides, at least 80 nucleotides, at least 85 nucleotides, at least 90 nucleotides, at least 95 nucleotides, at least 100 nucleotides, at least 105 nucleotides, at least 110 nucleotides, at least 115 nucleotides, at least 120 nucleotides, at least 125 nucleotides, at least 130 nucleotides, at least 135 nucleotides, at least 140 nucleotides, at least 145 nucleotides, at least 150 nucleotides, at least 155 nucleotides, at least 160 nucleotides, at least 165 nucleotides, at least 170 nucleotides, at least 175 nucleotides, at least 180 nucleotides, at least 185 nucleotides, at least 190 nucleotides, at least 195 nucleotides, at least 200 nucleotides, at least 205 nucleotides, at least 210 nucleotides, at least 215 nucleotides, at least 220 nucleotides, at least 225 nucleotides, at least 230 nucleotides, at least 235 nucleotides, at least 240 nucleotides. The length of the consecutive adenine nucleotides in the polyA sequence may be in a range of 30-240 nucleotides, such as 40-230 nucleotides, 45-220 nucleotides, 50-210 nucleotides, 60-200 nucleotides, 70-190 nucleotides, 80-180 nucleotides, 90-170 nucleotides, 100-160 nucleotides, 110-150 nucleotides, 120-140 nucleotides. In some embodiments, the polyA sequence may consist only of consecutive adenine nucleotides.

The circular RNA may be in the range of about 500 to about 10,000 nucleotides. In some embodiments, the circular RNA may be at least 500, 550, 600, 650, 700, 750, 800, 850, 900, 950, 1,000, 1,100, 1,200, 1,300, 1,400, 1,500, 1,600, 1,700, 1,800, 1,900, 2,000, 2,500, 3,000, 3,500, 4,000, 4,500 or 5,000 nucleotides in size. In some embodiments, the circular RNA is no more than 10,000, 9,000, 8,000, 7,000, 6,000, 5,000 or 4,000 nucleotides in size.

The circular RNA of the present invention is translatable, that is, it can be translated to a protein, e.g., in an in vitro system or in a cell. The cell may be a eukaryotic cell or a prokaryotic cell. In some embodiments, the circular RNA is a mRNA.

In some embodiments, the circular RNA of the present invention does not comprise a UTR, such as a 5′UTR and/or a 3′UTR.

The circular RNA may comprise additional elements which may not impede the translation of protein from the circular RNA. In some embodiments, the additional elements may facilitation the production of the circular RNA or the translation of the protein from the circular.

The circular RNA of the present invention may be prepared by general strategies for RNA circularization methods, such as chemical methods using cyanogen bromide or a similar condensing agent, enzymatic methods using RNA or DNA ligases, and ribozymatic methods using self-splicing introns (Petkovic, S. & Muller, S., “RNA circularization strategies in vivo and in vitro”, Nucleic Acids Research, 43(4): 2454-2465 (2015); Beadudry, D. & Perreault, J., “An efficient strategy for the synthesis of circular RNA molecules”, Nucleic Acids Research, 23(15): 3064-3066 (1995); Micura, R., “Cyclic Oligoribonucleotides (RNA) by Solid-Phase Synthesis”, Chemistry A European Journal, 5(7): 2077-2082 (1999)).

In some embodiments, the circular RNA of the present invention may be prepared by circularizing a precursor RNA molecule. The circularization of the precursor RNA molecule may be performed by a ribozymatic method using self-splicing introns.

The term “precursor RNA”, as used herein, refers to an RNA sequence that is circularized to form the circular RNA of the present invention.

The precursor RNA may be linear. The precursor RNA may comprise a circularization unit and at least one circularizing element. The term “circularization unit”, as used herein, refers to the sequence to be circularized and the sequence that will be comprised in the circular RNA by circularizing the precursor RNA. The term “circularizing element”, as used herein, refers to a nucleic acid sequence that can be manipulated or be spontaneously spliced and ligated under suitable conditions to circularize a nucleic acid sequence adjacent to the circularizing element.

The circularization unit comprises at least, in the following order, a IRES element, a protein coding sequence and a polyA, and optionally additional elements.

The circularizing element may be positioned on either side or both sides of the circularization unit.

In some embodiments, the circularizing element may comprise intron self-splicing sequences from Group I or Group II.

In some embodiments, the intron self-splicing sequences comprises a first intron sequence on the 5′ of the circularization unit and a second intron sequence on the 3′ of the circularization unit.

In some embodiments, the intron self-splicing sequences may be derived from Group I permuted intron-exon (PIE) sequences, wherein the first intron sequence may comprise a 3′ group I intron fragment and the second intron sequence may comprise a 5′ group I intron fragment. The group I permuted intron-exon (PIE) sequences may be derived from T4 bacteriophage gene td or Cyanobacterium anabaena sp. pre-tRNA-Leu gene. In one embodiment, the 3′ group I intron fragment and/or the 5′ group I intron fragment is from a Cyanobacterium anabaena sp. pre-tRNA-Leu gene or T4 phage Td gene.

In some embodiments, the 3′ group I intron fragment” has 75% or higher sequence identity (such as 80%, 85%, 90%, 95% or 100%) to the 3′ proximal end of a natural group I intron, including the splice site dinucleotide and optionally the adjacent exon sequence. The adjacent exon sequence may have at least 1 nucleotide in length (e.g., at least 5 nucleotides in length, at least 10 nucleotides in length, at least 15 nucleotides in length, at least 20 nucleotides in length, at least 25 nucleotides in length, or at least 30 nucleotides in length). In some embodiments, the 3′ group I intron fragment is as set forth in SEQ ID NO: 588.

In some embodiments, the “5′ group I intron fragment” has 75% or higher sequence identity (such as 80%, 85%, 90%, 95% or 100%) to the 3′ proximal end of a natural group I intron, including the splice site dinucleotide and optionally the adjacent exon sequence. The adjacent exon sequence may have at least 1 nucleotide in length (e.g., at least 5 nucleotides in length, at least 10 nucleotides in length, at least 15 nucleotides in length, at least 20 nucleotides in length, at least 25 nucleotides in length, or at least 30 nucleotides in length). In some embodiments, the 5′ group I intron fragment is as set forth in SEQ ID NO: 592.

The term “splice site”, as used herein, refers to a dinucleotide that is included in a group I intron and between which a phosphodiester bond is cleaved during RNA circularization.

During the circularization of the precursor RNA comprising Group I intron self-splicing sequences, the precursor RNA undergoes the double trans esterification reactions characteristic of group I catalytic introns. Firstly, 3′ OH of a free guanine nucleoside (or one located in the intron) or a nucleotide cofactor (GMP, GDP, GTP) attacks phosphate at the splice site in the 5′ group I intron fragment and results in a break. Then the 3′OH of the break attacks phosphate at the splice site in the 3′ group I intron fragment and triggers the second transesterification, thereby joining the circularization unit together.

The precursor RNA may further comprise additional elements, such as elements that can facilitate the circularization of the precursor RNA and/or the translation of the protein coding region, such as spacers and/or homology arms.

The term “spacer”, as used herein, refers to any contiguous nucleotide sequence separating two other elements along a polynucleotide sequence. The spacer is predicted to or can avoid interfering with the structure of the proximal structures, for example, from the IRES, the protein coding region, the polyA or the circularizing element. The spacer sequences may be used to allow these structures to fold independently and correctly, and promote the circularization of the precursor RNA. The spacer may be located adjacent to the circularizing element, the IRES, the protein coding region and/or the polyA. For example, the spacer may be located downstream of and adjacent to the first intron sequence and/or upstream of and adjacent to the second intron sequence. A spacer is typically non-coding.

The precursor RNA may further comprise one or more, such as two spacers. In some embodiments, the precursor RNA may comprise a 5′ spacer sequence between the first intron sequence and the internal ribosome entry site (IRES) sequence, and/or a 3′ spacer sequence between the polyA and the second intron sequence.

The 5′ spacer sequence may be at least 10 nucleotides in length. In some embodiments, the 5′spacer sequence is at least 10, 11, 12, 13, 14, 15, 16, 17, 18, 19, 20, 25 or 30 nucleotides in length. In some embodiments, the 5′ spacer sequence is no more than 100, 90, 80, 70, 60, 50, 45, 40, 35 or 30 nucleotides in length. In some embodiments the 5′spacer sequence is between 20 and 50 nucleotides in length. In some embodiments, the 5′spacer sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length. In some embodiments, the 5′ spacer sequence contains less than 30 consecutive adenosines. In some embodiments, the 5′ spacer is as set forth in SEQ ID NO: 589.

The 3′ spacer sequence may be at least 10 nucleotides in length. In some embodiments, the 3′spacer sequence is at least 10, 11, 12, 13, 14, 15, 16, 17, 18, 19, 20, 25 or 30 nucleotides in length. In some embodiments, the 3′spacer sequence is no more than 100, 90, 80, 70, 60, 50, 45, 40, 35 or 30 nucleotides in length. In some embodiments the 3′spacer sequence is between 20 and 50 nucleotides in length. In some embodiments, the 3′spacer sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length. In some embodiments, the 3′ spacer sequence contains less than 30 consecutive adenosines. In some embodiments, the 3′ spacer is as set forth in SEQ ID NO: 591.

Homology arms are generally used in pairs, and generally located external to the first intron sequence (i.e., on its 5′) and the second intron sequence (i.e., on its 3′). In some embodiments, the precursor RNA may comprise a 5′ homology arm on the 5′ of the first intron sequence and a 3′ homology arm on the 3′ of the second intron sequence.

The term “homology arm”, as used herein, refers to any contiguous sequence that is used to form base pairs with at least about 75% (e.g., at least about 80%, at least about 85%, at least about 90%, at least about 95%, about 100%) of another sequence in the RNA, such as another homology arm. The homology arm is generally located adjacent to the circularizing element, and can bring the first intron sequence and second intron sequence in close spatial proximity through base pairing, thereby facilitating the splicing of the introns and promoting the circularization or the precursor RNA. The homology arm generally tends not to form base pairs with unintended sequences in the RNA (e.g., sequences other than the homology arm). The homology arm may have less than 50% (e.g., less than 45%, less than 40%, less than 35%, less than 30%, less than 25%, less than 20%, less than 15%, less than 10% or less than 5%) sequence identity with the unintended sequences in the RNA.

The 5′ homology arm may be about 5-50 nucleotides in length. In some embodiments, the 5′ homology arm may be about 20-40 nucleotides in length. In some embodiments, the 5′ homology arm is at least 15, 16, 17, 18, 19, 20, 21, 22, 23, 24 or 25 nucleotides in length. In some embodiments, the 5′ homology arm is no more than 50, 45, 40, 39, 38, 37, 36, 35, 34, 33, 32, 31 or 30 nucleotides in length. In some embodiments, the 5′ homology arm is 20, 21, 22, 23, 24, 25, 26, 27, 28, 29, 30, 31, 32, 33, 34, 35, 36, 37, 38, 39 or 40 nucleotides in length. In some embodiments, the 5′ homology arm is as set forth in SEQ ID NO: 587.

The 3′ homology arm may be about 5-50 nucleotides in length. In some embodiments, the 3′ homology arm may be about 20-40 nucleotides in length. In some embodiments, the 3′ homology arm is at least 15, 16, 17, 18, 19, 20, 21, 22, 23, 24 or 25 nucleotides in length. In some embodiments, the 3′ homology arm is no more than 50, 45, 40, 39, 38, 37, 36, 35, 34, 33, 32, 31 or 30 nucleotides in length. In some embodiments, the 3′ homology arm is 20, 21, 22, 23, 24, 25, 26, 27, 28, 29, 30, 31, 32, 33, 34, 35, 36, 37, 38, 39 or 40 nucleotides in length. In some embodiments, the 3′ homology arm is as set forth in SEQ ID NO: 593.

In some embodiments, one or more elements in the precursor RNA or the circular RNA have a sequence identity of at least 75% (e.g., at least 80%, at least 85%, at least 90%, at least 95% or 100%) with natural sequences, including e.g., the IRES and the intron fragment. In some embodiments, the protein coding sequence is not naturally occurring nucleotide sequence. In some embodiments, the protein coding region encodes a natural or a synthetic protein.

The precursor RNA may be circularized under suitable conditions, which depend on the specific circularizing strategy and are known to those skilled in the art. For example, The condition for circularizing a precursor RNA comprising Group I intron self-splicing sequences may be in the presence of magnesium ions and quanosine nucleotide or nucleoside and under a temperature at which RNA circularization occurs (e.g., between about 20° C. and about 60° C.).

The circularization of the precursor RNA may be performed in vitro. Alternatively, the circularization of the precursor RNA may be performed in a cell, wherein the precursor RNA may be introduced into a cell or a DNA template for the precursor RNA may be introduced into a cell to be transcribed to the precursor RNA, then the precursor RNA is circularized in the cell.

The precursor RNA of the present invention may be artificially synthesized or be obtained by transcription from a DNA template.

The DNA template may be comprised in a vector.

The vector of the present invention may be an expression vector, The term “expression vector” refers to a vector designed to enable the expression of an inserted nucleic acid sequence.

Vectors may be introduced into the desired host cells by methods known in the art, e.g., transfection, electroporation, microinjection, transduction, cell fusion, DEAE dextran, calcium phosphate precipitation, lipofection (lysosome fusion), use of a gene gun, or a DNA vector transporter (see, e.g., Wu et al., J. Biol. Chem. 267:963 (1992); Wu et al., J. Biol. Chem. 263:14621 (1988); and Hartmut et al., Canadian Patent Application No. 2,012,311).

The vector of the present invention may be a DNA construct, such as a plasmid, or a viral vector.

The vector of the present invention comprises a transcription unit, which is a polynucleotide sequence that can be transcribed to the precursor RNA. The vector may further comprise a promoter that initiate the transcription of the transcription unit. The promoter may be located upstream of and adjacent to the transcription unit. The promoter may be an RNA polymerase promoter. Examples of the RNA polymerase promoter include, but are not limited to a T7 RNA polymerase promoter, T6 RNA polymerase promoter, SP6 RNA polymerase promoter, T3 RNA polymerase promoter, or T4 RNA polymerase promoter.

The elements in the circular RNA, the precursor RNA or the vector of the present invention are operably linked to each other.

In some embodiments, the present invention relates to a method of producing a circular RNA, the method comprising generating precursor RNA by performing transcription using the vector provided herein as a template, and circularizing the precursor RNA to obtain the circular RNA.

The transcription step may be performed in vitro (i.e., in a cell-free system) or in a cell. The circularization step may be performed in vitro or in a cell. In vitro transcription methods are known to the skilled person. For example, there are a number of commercially available in vitro transcription kits.

In some embodiments, artificially synthesized precursor RNA is introduced into a host cell, and the precursor RNA is circularized in the cell to obtain the circular RNA. In some embodiments, the vector provided herein is introduced into a host cell and is transcribed in the cell to the precursor RNA, and the precursor RNA is circularized in the cell to obtain the circular RNA.

The circular RNA of the present invention may be purified by a variety of methods, such as a size-exclusion column. In preferred embodiments, the poly A comprised in the circular allows for purification by oligo dT-based capturing, such as by oligo dT beads or oligo dT resin, such as oligo dT affinity chromatography.

The term “oligo-dT”, as used herein, refers to a homopolymer consisting exclusively of thymidines. Oligo dT beads refers to magnetic beads that is conjugated to Oligo dT. Oligo dT resin refers to resin (such as Sepharose resin) that is covalently conjugated to Oligo dT.

In some embodiments, the present invention relates to a method of purifying a circular RNA, the method comprising producing the circular RNA provided herein, and purifying the circular RNA through oligo dT-based capturing.

In some embodiments, the present invention relates to a method of expressing a protein in a cell. Said method may comprise introducing the circular RNA, the vector or the precursor RNA provided herein into a host cell and expressing the protein encoded by the protein coding sequence in the circular RNA.

The circular RNA, the vector or the precursor RNA may be introduced into the host cell by methods known in the art, e.g., electroporation, microinjection, transduction, cell fusion, DEAE dextran, calcium phosphate precipitation, lipofection (lysosome fusion), or use of a gene gun.

In some embodiments, in order to express protein in a cell, the circular RNA may be introduced into the cell using, for example, lipofection or electroporation. In some embodiments, the circular RNA may be introduced into a cell using a nanocarrier which can be, for example, a lipid, a polymer or a lipo-polymeric hybrid, such as a lipid nanoparticle (LNP).

The host cell may be a prokaryotic or a eukaryotic cell. In some embodiments, the host cell may be a mammal cell, preferably a human cell, such as a T cell, a NK cell or a A549 cell.

The protein expressed from the circular RNA may be further purified. Methods for purifying a protein are well known to those skilled in the art.

In some embodiments, the present invention relates to a method of producing a protein, the method comprising expressing the protein from the circular RNA provided herein, and purifying the protein. The circular RNA may be produced by any method provided herein.

In some embodiments, the present invention relates to a cell or a cell population comprising the circular RNA, the precursor RNA or the vector provided herein. The cell may be a mammal cell, preferably a human cell, more preferably a T cell or a NK cell.

The circular RNA, the precursor RNA, the vector, the cell or the cell population can be used to express a protein of interest, produce a protein of interest or treat a disease via the expressed therapeutic protein. The circular RNA, the precursor RNA, the vector, the cell or the cell population can also be used as a vaccine.

The cell or a cell population may be an immune effector cell, such as a T cell, a NK cell, an NKT cell, a macrophage, a neutrophil, or a granulocyte cell, or a population comprising these cells. In some embodiments, the T cell is a cytotoxic T cell, a helper T cell, a gamma delta T, a CD4+/CD8+ double positive T cell, a CD4+ T cell, a CD8+ T cell, a CD4/CD8 double negative T cell, a CD3+ T cell, a naive T cell, an effector T cell, a helper T cell, a memory T cell, a regulator T cell, a Th0 cell, a Th1 cell, a Th2 cell, a Th3 (Treg) cell, a Th9 cell, a Th17 cell, a Thαβ helper cell, a Tfh cell, a stem memory TSCM cell, a central memory TCM cell, an effector memory TEM cell, or an effector memory TEMRA cell.

In some embodiments, the present invention provides to a cell or a cell population comprising a first polynucleotide encoding a CAR and a second polynucleotide encoding a fusion protein comprising a first domain that activates an antigen-presenting cell (APC) and a second domain that activates an immune effector cell, wherein the first polynucleotide and the second polynucleotide are located on same or different circular RNAs in the cell, wherein (i) the first domain comprises (a) a ligand that binds to an activation receptor of the APC, or a receptor-binding fragment thereof, or (b) an antibody that binds to an activation receptor of the APC, or an antigen-binding fragment thereof; and (ii) the second domain comprises (a) a co-stimulatory receptor of the immune effector cell, or a functional fragment thereof, (b) a co-stimulatory ligand of the immune effector cell, or a receptor-binding fragment thereof, or (c) an antibody that binds to a co-stimulatory receptor of the immune effector cell, or an antigen-binding fragment thereof.

The first polynucleotide and the second polynucleotide function as protein coding sequences in the circular RNA. The first polynucleotide may encode the first protein described in the present invention. The second polynucleotide may encode the second protein described in the present invention.

When the first polynucleotide and the second polynucleotide are located on same circular RNA, the first polynucleotide may be positioned in the 5′ end or the 3′ end of the second polynucleotide. The first polynucleotide and the second polynucleotide can be linked by a nucleotide sequence encoding a linker. The linker can be a self-cleaving linker, such as a 2A peptide (e.g., P2A, F2A, T2A etc.).

The CAR encoded by the first polynucleotide may be any CAR described herein. The fusion protein encoded by the second polynucleotide may be any fusion protein in the context of LACOSTIM described herein.

The present invention also relates to a composition, e.g., a pharmaceutical composition, which comprises the circular RNA, the precursor RNA, the vector or the cell provided herein and optionally a carrier. The carrier may be a delivery carrier or a pharmaceutical acceptable carrier (such as a pharmaceutical acceptable delivery carrier). The pharmaceutical composition may be a vaccine composition. The vaccine composition may comprise the circular RNA, the precursor RNA, the vector, the cell or the cell population, and an adjuvant (e.g., aluminum hydroxide, BCG, etc.).

The pharmaceutical composition of the present invention may be formulated for a variety of means of administration in accordance with known techniques. See, for example, Remington, The Science and Practice of Pharmacy (9th Ed. 1995). In the manufacture of a pharmaceutical composition, the active agent is typically admixed with, inter alia, a pharmaceutical acceptable carrier. The pharmaceutical acceptable carrier must, of course, be acceptable in the sense of being compatible with any other ingredients in the formulation and must not be deleterious to the subject. A pharmaceutically acceptable carrier may include, but is not limited to, a buffer, an excipient, a stabilizer, a preservative, a wetting agent, a surfactant, an emulsifying agent, or combinations thereof. Examples of a buffer include, but is not limited to acetic acid, citric acid, histidine, boric acid, formic acid, succinic acid, phosphoric acid, carbonic acid, malic acid, aspartic acid, Tris buffer, HEPPSO, HEPES, neutral buffered saline, phosphate buffered saline and the like.

The term “delivery carrier”, as used herein, refers to a carrier for delivering a DNA or RNA molecular to a target cell or a target tissue where the protein of interest is expressed. Examples of the delivery carrier include, but are not limited to, lipid nanoparticles or polymers.

The present invention also relates to a composition, e.g., a pharmaceutical composition, which comprises a cell that comprises the circRNA of the present invention. The cell may be an immunologic effector cell, such as a T cell, a NK cell, an NKT cell, a macrophage, a neutrophil, or a granulocyte cell.

The pharmaceutical composition of the present invention can be used to treat or prevent a disease, e.g., tumor, cancer, a virus infection or an autoimmune disease, in a subject. In some embodiments, the cancer is a solid tumor or a hematological cancer (such as leukemia). In some embodiments, the cancer is acute myeloid leukemia (AML), B-acute lymphoid leukemia (B-ALL), T-acute lymphoid leukemia (T-ALL), B cell precursor acute lymphoblastic leukemia (BCP-ALL) or blastic plasmacytoid dendritic cell neoplasm (BPDCN). In some embodiments, the disease (such as cancer) is characterized in that the disease cell expresses mesothelin, CD123, BCMA, HER2, IL13Ra2, B7H3. In some embodiments, the cancer is CD123-expressing cancer. In some embodiments, the cancer is CD123-expressing AML. In some embodiments, the cancer is mesothelioma. In some embodiments, the mesothelioma is pleural mesothelioma, peritoneal mesothelioma, or pericardial mesothelioma. In some embodiments, the cancer is pancreatic cancer. In some embodiments, the pancreatic cancer is pancreatic ductal carcinoma. In some embodiments, the cancer is ovarian cancer. In some embodiments, the cancer is ovarian epithelial carcinoma. In some embodiments, the cancer is a lung cancer. In some embodiments, the cancer is non-Hodgkin's lymphoma, chronic lymphocytic leukemia, acute lymphocytic leukemia, human B-cell precursor leukemia, multiple myeloma, malignant lymphoma. In some embodiments, the cancer is breast cancer, stomach cancer, ovarian cancer, cervical cancer, uroepithelial cancer, esophageal cancer, bladder cancer, colorectal cancer, endometrial cancer, kidney cancer, lung cancer, pancreatic cancer, head and neck cancer, sarcoma, glioblastoma, prostate cancer, or thyroid cancer. In some embodiments, the cancer is a glioma or a head and neck cancer.

The present invention also relates to a method of treating a disease, the method comprising administering a therapeutically effective amount of a composition provided herein to a subject in need thereof. In some embodiments, the subject is a human.

The disease may be a tumor, cancer, a virus infection or an autoimmune disease. The disease may involve loss or absence of a functional protein which is encoded by the protein coding sequence in the circular RNA or high expression of a protein. In some embodiments, the disease may be a tumor with high expression of mesothelin, such as mesothelioma, pancreatic adenocarcinoma, ovarian cancer and/or lung adenocarcinoma. “High expression” means that the expression level of a tumor antigen or a tumor surface receptor on said tumor cell is higher than that on normal cells, e.g., 10%, 20%, 30%, 40%, 50%, 60%, 70%, 80%, 90% or more higher than that on normal cell.

The pharmaceutical composition of the present invention may be administered in any manner suitable to the disease to be treated (or prevented) and the subject. In certain embodiments, the administration manner may include, but is not limited to, parenteral or non-parenteral route, including oral, sublingual, buccal, percutaneous, rectal, vaginal, intradermal, intranasal route or parenteral route such as intravenous (i.v.), intraperitoneal, intradermal, subcutaneous, intramuscular, intracranial, intrathecal, intratumoral, transdermal, transmucosal intraarticular, intra-synovial, intrasternal, intrathecal, intrahepatic, intralesional or intracranial injection or infusion. The pharmaceutical compositions may be injected, for instance, directly into a tumor, lymph node, tissue, organ, or site of infection.

Dosage forms suitable for oral administration include, but are not limited to, tablet, capsule, powder, pill, granule, suspension, solution or preconcentrate of solution, emulsion or preconcentrates of emulsion. Pharmaceutical acceptable carriers that can be used in an oral dosage form include water, glycols, oils, alcohols, flavoring agents, preservatives, coloring agents and the like. Carriers such as starches, sugars, microcrystalline cellulose, diluents, filler, glidants, granulating agents, lubricants, binders, stabilizers, disintegrating agents and the like can be used to prepare an oral solid preparation such as powder, capsule or tablet.

Dosage forms suitable for parenteral administration include, but are not limited to, sterile liquid preparations, e.g., isotonic aqueous solutions, emulsions, suspensions, dispersions, or viscous compositions, which may be buffered to a desirable pH. Parenteral dosage forms may be ready for use or dry products ready to be dissolved or suspended in a pharmaceutically acceptable carrier. Parenteral dosage forms may be formulated sterile or are capable of being sterilized prior to administration to a subject. Pharmaceutical acceptable carriers that can be used to provide parenteral dosage forms include, but are not limited to, water for injection; aqueous vehicles such as, but not limited to, sodium chloride injection, Ringer's injection, dextrose injection; water-miscible carriers such as, but not limited to, ethyl alcohol, polyethylene glycol, and polypropylene glycol; non-aqueous carriers such as, but not limited to, corn oil, cottonseed oil, peanut oil, sesame oil, ethyl oleate, isopropyl myristate, and benzyl benzoate; and solubilizing agent such as cyclodextrin.

The quantity and frequency of administration will be determined by such factors as the condition of the subject (e.g., age, body weight, sex, and response of the subject to the medicament), and the type and severity of the subject's disease, although appropriate dosages may be determined by clinical trials.

The pharmaceutical composition may be administered to a subject in a therapeutically effective amount of about 0.5 to about 250 mg/kg, e.g., about 1 to about 250 mg/kg, about 2 to about 200 mg/kg, about 3 to about 120 mg/kg, about 5 to about 250 mg/kg, about 10 to about 200 mg/kg, or about 20 to about 120 mg/kg.

The pharmaceutical composition may be administered once or twice one day; or once every 2, 3, 4, 5, 6, 7, 8, 9 or 10 days, once every 1, 2, 3, 4, 5, or 6 weeks or once every 1, 2, 3, 4, 5, or 6 months or longer. The pharmaceutical composition may also be administered in a several times (e.g., 1, 2, 3, 4 or 5 times) weekly or a several times (e.g., 1, 2, 3, 4, 5, 6, 7, 8, 9 or 10 times) monthly scheme. for example, in a five times weekly scheme, the pharmaceutical composition may be administered once daily for five consecutive days followed by two consecutive days off.

The circular RNA, the precursor RNA or the vector can be used to improve protein expression level. Therefore, the present invention also relates to a method of improving protein expression level of a circular RNA, the method comprising inserting a polyA in the circular RNA. The polyA is defined as described above. The circular RNA comprising the inserted polyA sequence has higher protein expression level compared with circular RNA counterpart without the polyA sequence (i.e., a circular RNA does not comprise the polyA sequence as describe herein).

### EXAMPLES

### Example 1

### Methods

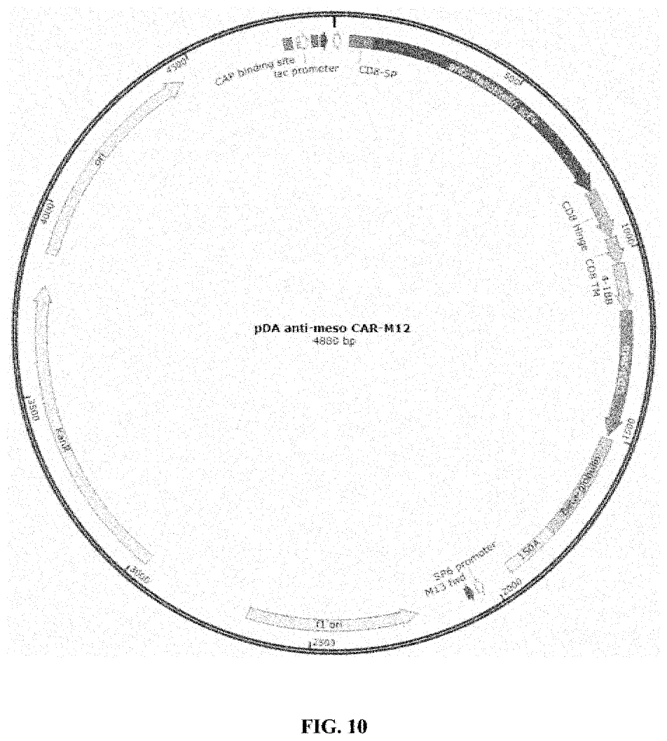

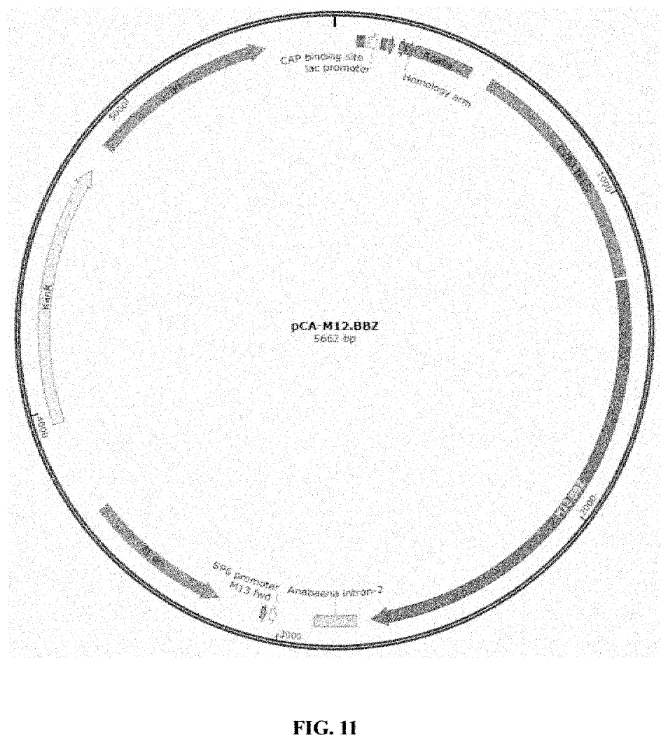

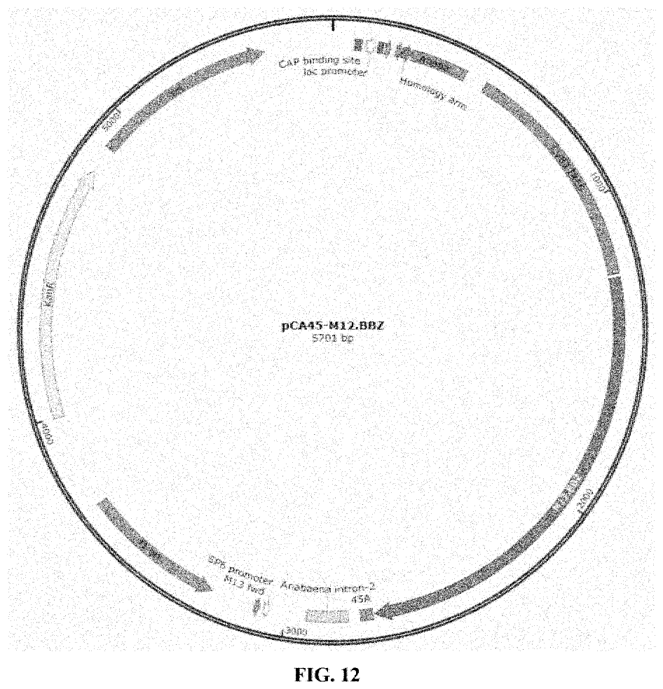

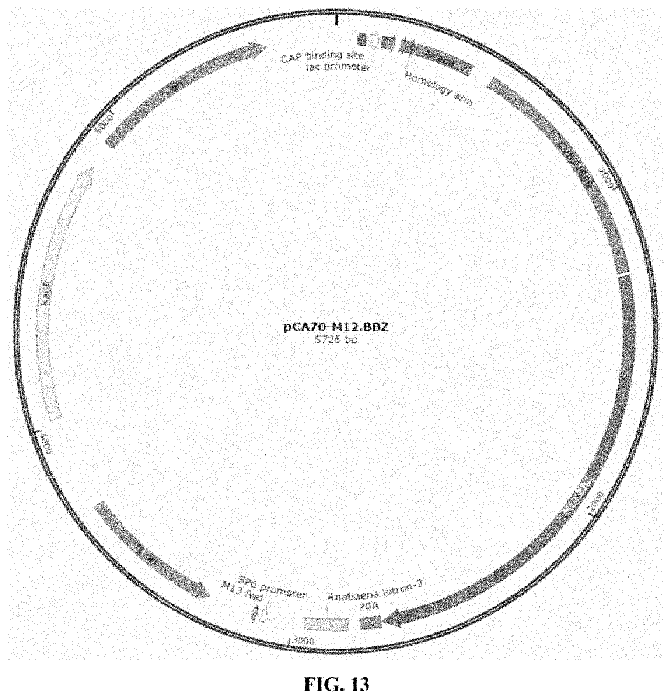

1. In Vitro Transcription (IVT) of Anti-MSLN M12 CAR Linear mRNA
1.1 Linearize the pDA-M12.BBZ CAR plasmid (FIG. 10) by digestion with BspQ1 enzyme.
1.2 Purify the linearized vector by PCR Cleanup kit (Qiagen), and elute with RNase-free water.
1.3 The concentration of DNA is measured by nanodrop, and checked by running agarose DNA gel.
1.4 IVT is performed following the manufacturer' protocol (Thermofisher, Cat No: AMB13455). Briefly, 1 μg template DNA, NTP/ARCA buffer, T7 buffer, GTP, T7 enzyme and RNase free H₂O were added in 20 μl volume to 0.2 ml PCR tube, and incubate at 37° C. for 4 hours.
1.5 4 hours later, add 2 μl of DNase I per reaction, and incubate at 37° C. for 15 min.
1.6 Then perform tailing procedure according to the manufacturer's suggestion.
1.7 Purify the IVT mRNA using the RNasy kit (Qiagen).
1.8 The concentration of RNA is measured by nanodrop, and checked by running PAGE gel.
   2. In Vitro Transcription (IVT) of Anti-MSLN M12 CAR circRNA
2.1 Linearize the pCA-M12.BBZ, pCA45-M12.BBZ, pCA70-M12.BBZ CAR plasmid (FIG. 11-13) by digestion with BspQ1 enzyme.
2.2 Purify the linearized vector by PCR Cleanup kit (Qiagen), and elute with RNase-free water.
2.3 The concentration of DNA is measured by nanodrop, and checked by running agarose DNA gel.
2.4 IVT is performed following the manufacturer's protocol (Thermofisher, Cat No: AMB13345). Briefly, 1 μg template DNA, NTP mixture, T7 buffer, GTP, T7 enzyme and RNase free H₂O were added in 20 μl volume to 0.2 ml PCR tube, and incubate at 37° C. for 4 hours.
2.5 4 hours later, add 2 μl of DNase I per reaction, and incubate at 37° C. for 15 min.
2.6 After DNase treatment additional GTP was added to a final concentration of 2 mM, and then reactions were heated at 55° C. for 15 min.
2.7 circRNA was then purified by column or AKTA system.
2.8 The concentration of RNA is measured by nanodrop, and checked by running PAGE gel.
   3. Purify circRNA by CIMmultus Oligo dT Column
3.1 Buffer preparation: Buffer A: Equilibration/wash buffer. 50 mM sodium phosphate, 250 mM sodium chloride, 5 mM EDTA, pH 7.0; Buffer B. Second wash buffer. 50 mM sodium phosphate, 5 mM EDTA, pH 7.0; Buffer C. Elution buffer. 10 mM Tris, pH 8.0; Buffer D. Cleaning buffer. 3 M Guanidine-HCl, 5 mM EDTA, pH 7.0.
3.2 Equilibrate column with buffer A: Pump equilibration buffer (A) through the CIMmultus Oligo dT column until output pH and conductivity are the same as the input buffer. Flow rate: 5 column volumes per minute (CV/min).
3.3 Inject sample. Perform sample injection according to the manufacturer's suggestions.
3.4 Wash1 with buffer A: 10 CV of equilibration buffer.
3.5 Wash2 with buffer B: 10 CV second wash buffer.
3.6 Elute with buffer C: elute and collect circRNA sample, until UV absorbance returns to baseline.
3.7 Clean with buffer D. clean and wash the column system with at least 10 CV of cleaning buffer.

### 4. Electroporation of RNA to A549-GFP and T Cells

4.1 Collect A549-GFP tumor cells and T cells and wash with Opti-MEM medium for 3 times.
4.2 Resuspend cell pellet with Opti-MEM medium, adjust the cell concentration to 1×10e7/ml.
4.3 Aliquot 5 μg MSLN mRNA to 1.5 ml EP tube, then add 100 μl A549 cells, mix well. For T cells electroporation, Aliquot 10 μg M12.BBZ mRNA or circRNA to 1.5 ml EP tube, then add 100 μl A549 cells, mix well.
4.4 Set up parameters on BTX machine:
a) for T cell: 500 voltage, 0.7 ms
b) for A549 tumor cell: 300 voltage, 0.5 ms;
4.5 Add 100 μl cells mixed with RNA in the BTX electroporation cup, tap to avoid bubble.
4.6 Perform electroporation, then transfer cells to the pre-warmed culture medium and culture at 37° C.

### 5. In Vitro Cytotoxicity Assay of Anti-MSLN M12 CAR-T Cells

5.1 12 hours before coculture, seed A549-EGFP cells that electroporated with 5 μg MSLN mRNA to flat-bottomed 96-well plate with 3000 cells/100 μl/well.
5.2 About 12 to 24 hours post electroporation, count and dilute CAR-T cells to appropriate concentration, seed cells with 100 ul/well to tumor cells with different E/T ratios, such as 3:1 and 1:1.
5.3 Put the co-culture plate into IncuCyte S3 machine, and setup scanning parameters.
5.4 After 4 days scanning, the Total Green Object Integrated Intensity (GCU×μm²/Well) was analyzed to calculate the killing efficiency.
   The Elements Used in this Example:
T7 promoter: SEQ ID NO: 586.
M12: anti-mesothelin (anti-MSLN) scFv.
M12.BBZ: a chimeric antigen receptor (CAR) comprising M12, CD8 hinge domain, CD8 transmembrane (TM) domain, 4-1BB costimulatory domain and CD3ζ domain, as shown in SEQ ID NO: 572.
3′UTR: SEQ ID NO: 597
HA-1: Homology arm-1, as shown in SEQ ID NO: 587.
Intron-1: Anabaena intron-1, as shown in SEQ ID NO: 588.
Spacer-1: SEQ ID NO: 589.
IRES: CVB3 IRES, as shown in SEQ ID NO: 590.
Spacer-2: SEQ ID NO: 591.
Intron-2: Anabaena intron-2, as shown in SEQ ID NO: 592.
HA-2: homology arm-2, as shown in SEQ ID NO: 593.

### Results

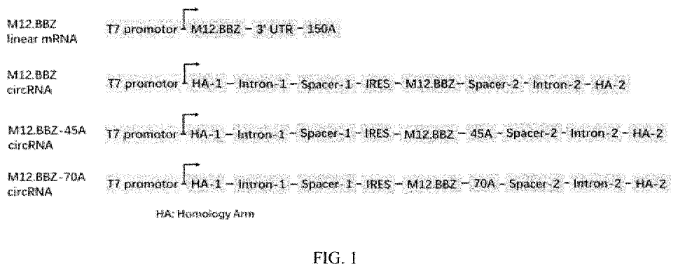

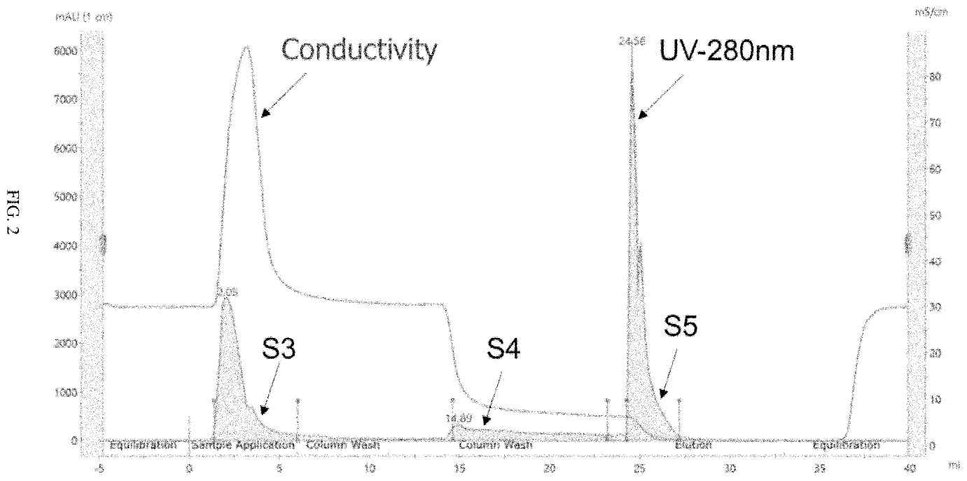

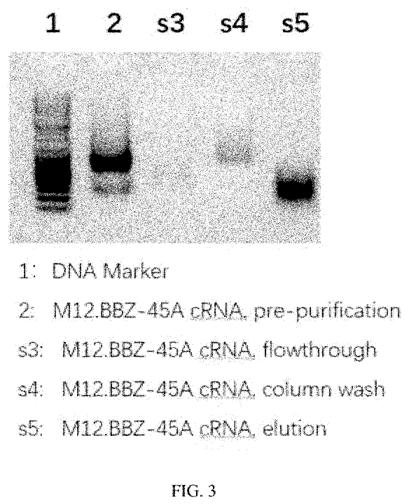

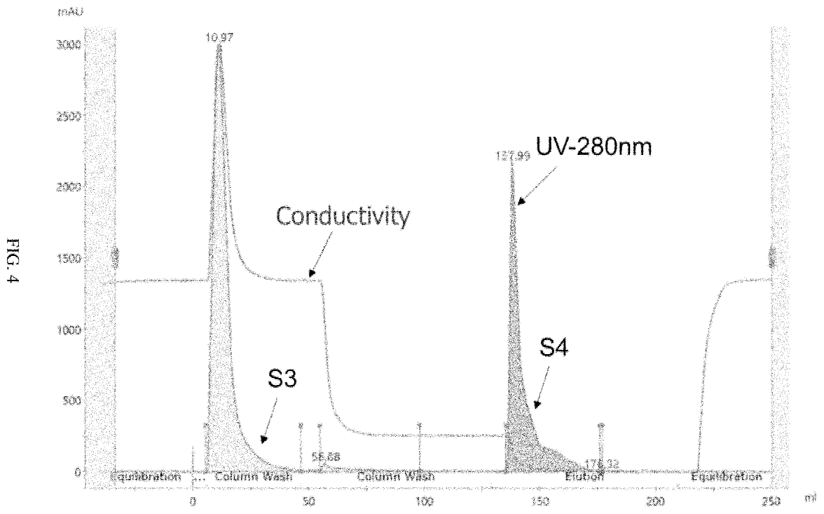

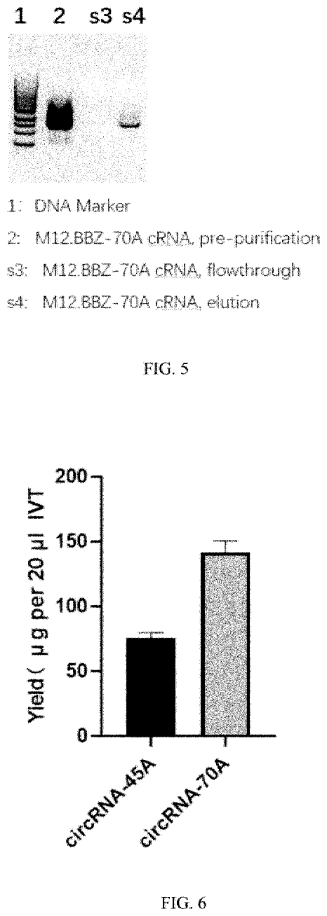

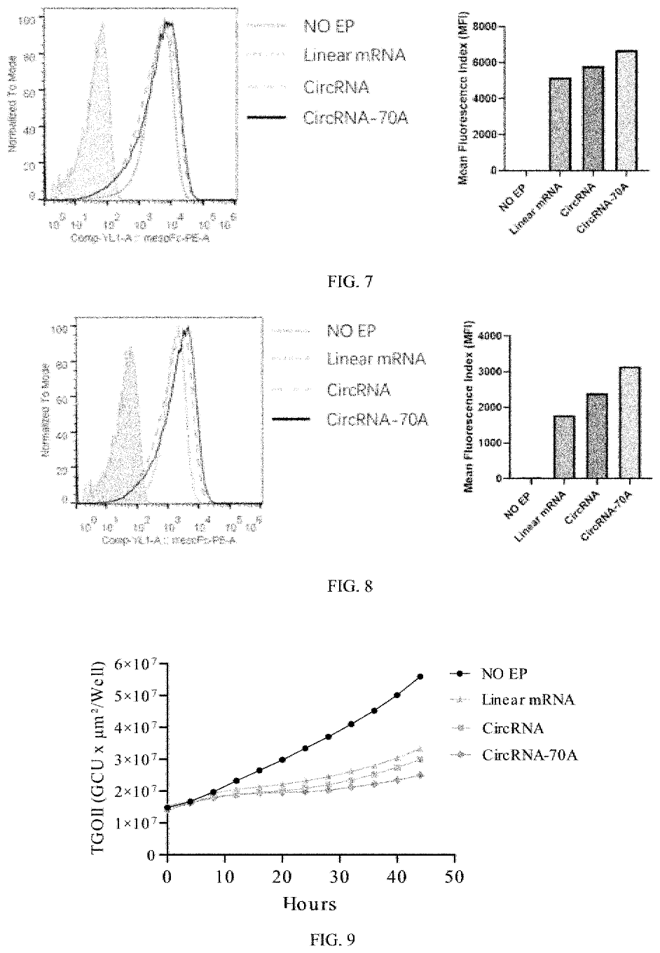

First, we construct circRNA vector with no polyA, poly 45A and poly 70A (FIG. 1), and perform IVT using these vectors. Then, we test whether circRNA with 45A and 70A can be purified by oligo dT resin, which is commonly used in linear mRNA purification. Results showed that circRNA with 45A and 70A can be successfully purified by oligo dT resin with high purity (FIG. 2-5). Moreover, the yield of circRNA-70A is higher than circRNA-45A (FIG. 6). To test the function of circRNA with poly A sequence, we transduce the same amount of M12.BBZ linear mRNA, circRNA with no polyA and poly 70A into activated T cells. We test the expression level of each RNA by flow cytometry staining of the M12 CAR with mesothelin-Fc recombinant protein in 24 hours (FIG. 7) and 48 hours (FIG. 8) post transduction, results showed that the higher expression level of M12 CAR in the circRNA-70A group than linear mRNA group and circRNA-45A group. Coculture assay using these CAR-T cells with mesothelin expressing tumor cells also demonstrated the higher killing ability of the circRNA-70A CAR-T group than linear mRNA CAR-T group and circRNA CAR-T group (FIG. 9).

### Sequences Used in Example 1

Anti-MSLN M12.BBZ CAR amino acid sequence (SEQ ID NO: 572):
(wherein the underlined amino acid sequences are, in their orders, LCDR1, LCDR2, LCDR3,
HCDR1, HCDR2, HCDR3, respectively; the italic amino acid sequence
(GGGGSGGGGSGGGGS) is a linker between VL and VH of anti-MSLN scFv; the underlined
and italic amino acid sequence is CD8 signal peptide)
MALPVTALLLPLALLLHAARPAIRLTQSPSLLSASVGDRVTVTCRASQGGGNYLAWYQQK
PGKAPKLLIYGASKLQSGVPSRFSGSGSGTEFTLTISSLQPEDFATYYCQQLNSYPVTFGQ
GTKVDIKGGGGSGGGGSGGGGSEVQLVESGAEVKKPGASVKVSCKASGYTFTTYYIHW
VRQAPGQGLEWMGIINPSSGSTTYTQKFQGRVTMTRDTSTSTVYIELSGLRSEDTAVYY
CARGETLRGYFDYWGQGTLVTVSSTTTPAPRPPTPAPTIASQPLSLRPEACRPAAGGAVH
TRGLDFACDIYIWAPLAGTCGVLLLSLVITLYCKRGRKKLLYIFKQPFMRPVQTTQEEDG
CSCRFPEEEEGGCELRVKFSRSADAPAYKQGQNQLYNELNLGRREEYDVLDKRRGRDP
EMGGKPRRKNPQEGLYNELQKDKMAEAYSEIGMKGERRRGKGHDGLYQGLSTATKDT
YDALHMQALPPR
Anti-MSLN M12.BBZ CAR nucleotide sequence (SEQ ID NO: 573):
atggccttaccagtgaccgccttgctcctgccgctggccttgctgctccacgccgccaggccggccatccggttgacccagtctccatccct
cctgtctgcatctgtaggagacagggtcaccgtcacttgtcgggccagtcagggcggtggcaattatttagcctggtatcagcaaaaaccag
ggaaagccccgaaactcctgatctatggtgcatccaagttgcaaagtggggtcccatcgaggttcagcggcagtggatctgggacagaatt
cactctcacaatcagcagtctgcagcctgaagattttgcaacttattactgtcaacagcttaatagttaccctgtcacttttggccaggggacca
aagtggatatcaaaggtggtggtggttctggcggcggcggctccggaggtggtggatccgaggtgcagctggtggagtctggggctgag
gtgaagaagcctggggcctcagtgaaggtttcctgcaaggcatctggatacaccttcaccacctactatatacactgggtgcgacaggccc
ctggacaagggcttgagtggatgggaataatcaaccctagtagtggtagcactacctacacacagaagttccagggcagagtcaccatgac
cagggacacgtccacgagcacagtctacattgaactgagcggcctgagatctgaagacacggccgtgtattactgtgcccgaggggaga
cgcttcggggctactttgactactggggccagggaaccctggtcaccgtctcttcaaccacgacgccagcgccgcgaccaccaacaccgg
cgcccaccatcgcgtcgcagcccctgtccctgcgcccagaggcgtgccggccagcggcggggggcgcagtgcacacgagggggctg
gacttcgcctgtgatatctacatctgggcgcccttggccgggacttgtggggtccttctcctgtcactggttatcaccctttactgcaaacgggg
cagaaagaaactcctgtatatattcaaacaaccatttatgagaccagtacaaactactcaagaggaagatggctgtagctgccgatttccaga
agaagaagaaggaggatgtgaactgagagtgaagttcagcaggagcgcagacgcccccgcgtacaagcagggccagaaccagctcta
taacgagctcaatctaggacgaagagaggagtacgacgttttggacaagagacgtggccgggaccctgagatggggggaaagccgaga
aggaagaaccctcaggaaggcctgtacaatgaactgcagaaagataagatggcggaggcctacagtgagattgggatgaaaggcgagc
gccggaggggcaaggggcacgatggcctttaccagggtctcagtacagccaccaaggacacctacgacgcccttcacatgcaggccct
gccccctcgctaa
M12 VL amino acid sequence (SEQ ID NO: 574):
AIRLTQSPSLLSASVGDRVTVTCRASQGGGNYLAWYQQKPGKAPKLLIYGASKLQSGVP
SRFSGSGSGTEFTLTISSLQPEDFATYYCQQLNSYPVTFGQGTKVDIK
M12 VH amino acid sequence (SEQ ID NO: 575):
EVQLVESGAEVKKPGASVKVSCKASGYTFTTYYIHWVRQAPGQGLEWMGIINPSSGSTT
YTQKFQGRVTMTRDTSTSTVYIELSGLRSEDTAVYYCARGETLRGYFDYWGQGTLVTV
SS
M12 LCDR1 (SEQ ID NO: 576):
RASQGGGNYLA
M12 LCDR2 (SEQ ID NO: 577):
GASKLQS
M12 LCDR3 (SEQ ID NO: 578):
QQLNSYPVT
M12 HCDR1 (SEQ ID NO: 579):
TYYIH
M12 HCDR2 (SEQ ID NO: 580):
IINPSSGSTTYTQKFQG
M12 HCDR3 (SEQ ID NO: 581):
GETLRGYFDY
DNA template for M12.BBZ linear mRNA (SEQ ID NO: 582):
taatacgactcactataggatggccttaccagtgaccgccttgctcctgccgctggccttgctgctccacgccgccaggccggccatccggt
tgacccagtctccatccctcctgtctgcatctgtaggagacagggtcaccgtcacttgtcgggccagtcagggcggtggcaattatttagcct
ggtatcagcaaaaaccagggaaagccccgaaactcctgatctatggtgcatccaagttgcaaagtggggtcccatcgaggttcagcggca
gtggatctgggacagaattcactctcacaatcagcagtctgcagcctgaagattttgcaacttattactgtcaacagcttaatagttaccctgtc
acttttggccaggggaccaaagtggatatcaaaggtggtggtggttctggcggcggcggctccggaggtggtggatccgaggtgcagctg
gtggagtctggggctgaggtgaagaagcctggggcctcagtgaaggtttcctgcaaggcatctggatacaccttcaccacctactatataca
ctgggtgcgacaggcccctggacaagggcttgagtggatgggaataatcaaccctagtagtggtagcactacctacacacagaagttcca
gggcagagtcaccatgaccagggacacgtccacgagcacagtctacattgaactgagcggcctgagatctgaagacacggccgtgtatta
ctgtgcccgaggggagacgcttcggggctactttgactactggggccagggaaccctggtcaccgtctcttcaaccacgacgccagegcc
gcgaccaccaacaccggcgcccaccatcgcgtcgcagcccctgtccctgcgcccagaggcgtgccggccagcggcggggggcgcag
tgcacacgagggggctggacttcgcctgtgatatctacatctgggcgcccttggccgggacttgtggggtccttctcctgtcactggttatca
ccctttactgcaaacggggcagaaagaaactcctgtatatattcaaacaaccatttatgagaccagtacaaactactcaagaggaagatggct
gtagctgccgatttccagaagaagaagaaggaggatgtgaactgagagtgaagttcagcaggagcgcagacgcccccgcgtacaagca
gggccagaaccagctctataacgagctcaatctaggacgaagagaggagtacgacgttttggacaagagacgtggccgggaccctgaga
tggggggaaagccgagaaggaagaaccctcaggaaggcctgtacaatgaactgcagaaagataagatggcggaggcctacagtgagat
tgggatgaaaggcgagcgccggaggggcaaggggcacgatggcctttaccagggtctcagtacagccaccaaggacacctacgacgc
ccttcacatgcaggccctgccccctcgctaagtcgacagctcgctttcttgctgtccaatttctattaaaggttcctttgttccctaagtccaac
tactaaactgggggatattatgaagggccttgagcatctggattctgcctaataaaaaacatttattttcattgctgcgtcgagagctcgctttc
ttgctgtccaatttctattaaaggttcctttgttccctaagtccaactactaaactgggggatattatgaagggccttgagcatctggattctgc
ctaataaaaaacatttattttcattgctgcctcgacgaattcaaaaaaaaaaaaaaaaaaaaaaaaaaaaaaaaaaaaaaaaaaaaaaaaaaaaa
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaa
aaaaaaa
DNA template for M12.BBZ circRNA (SEQ ID NO: 583)
taatacgactcactataggggagaccctcgaccgtcgattgtccactggtcaacaatagatgacttacaactaatcggaaggtgcagagact
cgacgggagctaccctaacgtcaagacgagggtaaagagagagtccaattctcaaagccaataggcagtagcgaaagctgcaagagaat
gaaaatccgttgaccttaaacggtcgtgtgggttcaagtccctccacccccacgccggaaacgcaatagccgaaaaacaaaaaacaaaaa
aaacaaaaaaaaaaccaaaaaaacaaaacacattaaaacagcctgtgggttgatcccacccacaggcccattgggcgctagcactctggt
atcacggtacctttgtgcgcctgttttataccccctcccccaactgtaacttagaagtaacacacaccgatcaacagtcagcgtggcacacca
gccacgttttgatcaagcacttctgttaccccggactgagtatcaatagactgctcacgcggttgaaggagaaagcgttcgttatccggccaa
ctacttcgaaaaacctagtaacaccgtggaagttgcagagtgtttcgctcagcactaccccagtgtagatcaggtcgatgagtcaccgcattc
cccacgggcgaccgtggcggtggctgcgttggcggcctgcccatggggaaacccatgggacgctctaatacagacatggtgcgaagag
tctattgagctagttggtagtcctccggcccctgaatgcggctaatcctaactgcggagcacacaccctcaagccagagggcagtgtgtcgt
aacgggcaactctgcagcggaaccgactactttgggtgtccgtgtttcattttattcctatactggctgcttatggtgacaattgagagatcgtt
accatatagctattggattggccatccggtgactaatagagctattatatatccctttgttgggtttataccacttagcttgaaagaggttaaaa
cattacaattcattgttaagttgaatacagcaaatctagagccaccatggccttaccagtgaccgccttgctcctgccgctggccttgctgctcc
acgccgccaggccggccatccggttgacccagtctccatccctcctgtctgcatctgtaggagacagggtcaccgtcacttgtcgggccagtca
gggcggtggcaattatttagcctggtatcagcaaaaaccagggaaagccccgaaactcctgatctatggtgcatccaagttgcaaagtggg
gtcccatcgaggttcagcggcagtggatctgggacagaattcactctcacaatcagcagtctgcagcctgaagattttgcaacttattactgtc
aacagcttaatagttaccctgtcacttttggccaggggaccaaagtggatatcaaaggtggtggtggttctggcggcggcggctccggaggt
ggtggatccgaggtgcagctggtggagtctggggctgaggtgaagaagcctggggcctcagtgaaggtttcctgcaaggcatctggatac
accttcaccacctactatatacactgggtgcgacaggcccctggacaagggcttgagtggatgggaataatcaaccctagtagtggtagca
ctacctacacacagaagttccagggcagagtcaccatgaccagggacacgtccacgagcacagtctacattgaactgagcggcctgagat
ctgaagacacggccgtgtattactgtgcccgaggggagacgcttcggggctactttgactactggggccagggaaccctggtcaccgtctc
ttcaaccacgacgccagcgccgcgaccaccaacaceggegcccaccatcgcgtcgcagcccctgtccctgcgcccagaggcgtgccg
gccagcggggggggcgcagtgcacacgagggggctggacttcgcctgtgatatctacatctgggcgcccttggccgggacttgtgggg
tccttctcctgtcactggttatcaccctttactgcaaacggggcagaaagaaactcctgtatatattcaaacaaccatttatgagaccagtacaa
actactcaagaggaagatggctgtagctgccgatttccagaagaagaagaaggaggatgtgaactgagagtgaagttcagcaggagcgc
agacgcccccgcgtacaagcagggccagaaccagctctataacgagctcaatctaggacgaagagaggagtacgacgttttggacaaga
gacgtggccgggaccctgagatggggggaaagccgagaaggaagaaccctcaggaaggcctgtacaatgaactgcagaaagataaga
tggcggaggcctacagtgagattgggatgaaaggcgagcgccggaggggcaaggggcacgatggcctttaccagggtctcagtacagc
caccaaggacacctacgacgcccttcacatgcaggccctgccccctcgctaagtcgacaaaaaacaaaaaacaaaacggctattatgcgtt
accggcgagacgctacggacttaaataattgagccttaaagaagaaattctttaagtggatgctctcaaactcagggaaacctaaatctagtta
tagacaaggcaatcctgagccaagccgaagtagtaattagtaagaccagtggacaatcgacggataacagcatatctag
DNA template for M12.BBZ-45A circRNA (SEQ ID NO: 584)
taatacgactcactataggggagaccctcgaccgtcgattgtccactggtcaacaatagatgacttacaactaatcggaaggtgcagagact
cgacgggagctaccctaacgtcaagacgagggtaaagagagagtccaattctcaaagccaataggcagtagcgaaagctgcaagagaat
gaaaatccgttgaccttaaacggtcgtgtgggttcaagtccctccacccccacgccggaaacgcaatagccgaaaaacaaaaaacaaaaa
aaacaaaaaaaaaaccaaaaaaaaaaacacattaaaacagcctgtgggttgatcccacccacaggcccattgggcgctagcactctggt
atcacggtacctttgtgcgcctgttttataccccctcccccaactgtaacttagaagtaacacacaccgatcaacagtcagcgtggcacacca
gccacgttttgatcaagcacttctgttaccccggactgagtatcaatagactgctcacgcggttgaaggagaaagcgttcgttatccggccaa
ctacttcgaaaaacctagtaacaccgtggaagttgcagagtgtttcgctcagcactaccccagtgtagatcaggtcgatgagtcaccgcattc
cccacgggcgaccgtggcggtggctgcgttggcggcctgcccatggggaaacccatgggacgctctaatacagacatggtgcgaagag
tctattgagctagttggtagtcctccggcccctgaatgcggctaatcctaactgcggagcacacaccctcaagccagagggcagtgtgtcgt
aacgggcaactctgcagcggaaccgactactttgggtgtccgtgtttcattttattcctatactggctgcttatggtgacaattgagagatcgtt
accatatagctattggattggccatccggtgactaatagagctattatatatccctttgttgggtttataccacttagcttgaaagaggttaaaa
cattacaattcattgttaagttgaatacagcaaatctagagccaccatggccttaccagtgaccgccttgctcctgccgctggccttgctgctcc
acgccgccaggccggccatccggttgacccagtctccatccctcctgtctgcatctgtaggagacagggtcaccgtcacttgtcgggccagtca
gggcggtggcaattatttagcctggtatcagcaaaaaccagggaaagccccgaaactcctgatctatggtgcatccaagttgcaaagtggg
gtcccatcgaggttcagcggcagtggatctgggacagaattcactctcacaatcagcagtctgcagcctgaagattttgcaacttattactgtc
aacagcttaatagttaccctgtcacttttggccaggggaccaaagtggatatcaaaggtggtggtggttctggcggcggcggctccggaggt
ggtggatccgaggtgcagctggtggagtctggggctgaggtgaagaagcctggggcctcagtgaaggtttcctgcaaggcatctggatac
accttcaccacctactatatacactgggtgcgacaggcccctggacaagggcttgagtggatgggaataatcaaccctagtagtggtagca
ctacctacacacagaagttccagggcagagtcaccatgaccagggacacgtccacgagcacagtctacattgaactgagcggcctgagat
ctgaagacacggccgtgtattactgtgcccgaggggagacgcttcggggctactttgactactggggccagggaaccctggtcaccgtctc
ttcaaccacgacgccagcgccgcgaccaccaacaccggegcccaccatcgcgtcgcagcccctgtccctgcgcccagaggcgtgccg
gccagcggggggggcgcagtgcacacgagggggctggacttcgcctgtgatatctacatctgggcgcccttggccgggacttgtgggg
tccttctcctgtcactggttatcaccctttactgcaaacggggcagaaagaaactcctgtatatattcaaacaaccatttatgagaccagtacaa
actactcaagaggaagatggctgtagctgccgatttccagaagaagaagaaggaggatgtgaactgagagtgaagttcagcaggagcgc
agacgcccccgcgtacaagcagggccagaaccagctctataacgagctcaatctaggacgaagagaggagtacgacgttttggacaaga
gacgtggccgggaccctgagatggggggaaagccgagaaggaagaaccctcaggaaggcctgtacaatgaactgcagaaagataaga
tggcggaggcctacagtgagattgggatgaaaggcgagcgccggaggggcaaggggcacgatggcctttaccagggtctcagtacagc
caccaaggacacctacgacgcccttcacatgcaggccctgccccctcgctaagtcgacaaaaaaaaaaaaaaaaaaaaaaaaaaaaaaa
aaaaaaaaaaaaaacaaaaaaaaaacggctattatgcgttaccggcgagacgctacggacttaaataattgagccttaaagaagaaattct
ttaagtggatgctctcaaactcagggaaacctaaatctagttatagacaaggcaatcctgagccaagccgaagtagtaattagtaagaccagt
ggacaatcgacggataacagcatatctag
DNA template for M12.BBZ-70A circRNA (SEQ ID NO: 585):
taatacgactcactataggggagaccctcgaccgtcgattgtccactggtcaacaatagatgacttacaactaatcggaaggtgcagagact
cgacgggagctaccctaacgtcaagacgagggtaaagagagagtccaattctcaaagccaataggcagtagcgaaagctgcaagagaat
gaaaatccgttgaccttaaacggtcgtgtgggttcaagtccctccacccccacgccggaaacgcaatagccgaaaaacaaaaaacaaaaa
aaacaaaaaaaaaaccaaaaaaaaaaacacattaaaacagcctgtgggttgatcccacccacaggcccattgggcgctagcactctggt
atcacggtacctttgtgcgcctgttttataccccctcccccaactgtaacttagaagtaacacacaccgatcaacagtcagcgtggcacacca
gccacgttttgatcaagcacttctgttaccccggactgagtatcaatagactgctcacgcggttgaaggagaaagcgttcgttatccggccaa
ctacttcgaaaaacctagtaacaccgtggaagttgcagagtgtttcgctcagcactaccccagtgtagatcaggtcgatgagtcaccgcattc
cccacgggcgaccgtggcggtggctgcgttggcggcctgcccatggggaaacccatgggacgctctaatacagacatggtgcgaagag
tctattgagctagttggtagtcctccggcccctgaatgcggctaatcctaactgcggagcacacaccctcaagccagagggcagtgtgtcgt
aacgggcaactctgcagcggaaccgactactttgggtgtccgtgtttcattttattcctatactggctgcttatggtgacaattgagagatcgtt
accatatagctattggattggccatccggtgactaatagagctattatatatccctttgttgggtttataccacttagcttgaaagaggttaaaa
cattacaattcattgttaagttgaatacagcaaatctagagccaccatggccttaccagtgaccgccttgctcctgccgctggccttgctgctcc
acgccgccaggccggccatccggttgacccagtctccatccctcctgtctgcatctgtaggagacagggtcaccgtcacttgtcgggccagtca
gggcggtggcaattatttagcctggtatcagcaaaaaccagggaaagccccgaaactcctgatctatggtgcatccaagttgcaaagtggg
gtcccatcgaggttcagcggcagtggatctgggacagaattcactctcacaatcagcagtctgcagcctgaagattttgcaacttattactgtc
aacagcttaatagttaccctgtcacttttggccaggggaccaaagtggatatcaaaggtggtggtggttctggcggcggcggctccggaggt
ggtggatccgaggtgcagctggtggagtctggggctgaggtgaagaagcctggggcctcagtgaaggtttcctgcaaggcatctggatac
accttcaccacctactatatacactgggtgcgacaggcccctggacaagggcttgagtggatgggaataatcaaccctagtagtggtagca
ctacctacacacagaagttccagggcagagtcaccatgaccagggacacgtccacgagcacagtctacattgaactgagcggcctgagat
ctgaagacacggccgtgtattactgtgcccgaggggagacgcttcggggctactttgactactggggccagggaaccctggtcaccgtctc
ttcaaccacgacgccagcgccgcgaccaccaacaccggcgcccaccategcgtcgcagcccctgtccctgcgcccagaggcgtgccg
gccagcggcggggggcgcagtgcacacgagggggctggacttcgcctgtgatatctacatctgggcgcccttggccgggacttgtgggg
tccttctcctgtcactggttatcaccctttactgcaaacggggcagaaagaaactcctgtatatattcaaacaaccatttatgagaccagtacaa
actactcaagaggaagatggctgtagctgccgatttccagaagaagaagaaggaggatgtgaactgagagtgaagttcagcaggagcgc
agacgcccccgcgtacaagcagggccagaaccagctctataacgagctcaatctaggacgaagagaggagtacgacgttttggacaaga
gacgtggccgggaccctgagatggggggaaagccgagaaggaagaaccctcaggaaggcctgtacaatgaactgcagaaagataaga
tggcggaggcctacagtgagattgggatgaaaggcgagcgccggaggggcaaggggcacgatggcctttaccagggtctcagtacagc
caccaaggacacctacgacgcccttcacatgcaggccctgccccctcgctaagtcgacaaaaaaaaaaaaaaaaaaaaaaaaaaaaaaa
aaaaaaaaaaaaaaaaaaaaaaaaaaaaaaaaaaaaaaaacaaaaaacaaaacggctattatgcgttaccggcgagacgctacggactta
aataattgagccttaaagaagaaattctttaagtggatgctctcaaactcagggaaacctaaatctagttatagacaaggcaatcctgagccaa
gccgaagtagtaattagtaagaccagtggacaatcgacggataacagcatatctag
T7 Promoter (SEQ ID NO: 586)
taatacgactcactatag
Homology arm-1 (SEQ ID NO: 587)
gggagaccctcgaccgtcgattgtccactggtc
Anabaena intron-1 (SEQ ID NO: 588)
aacaatagatgacttacaactaatcggaaggtgcagagactcgacgggagctaccctaacgtcaagacgagggtaaagagagagtccaat
tctcaaagccaataggcagtagcgaaagctgcaagagaatgaaaatccgttgaccttaaacggtcgtgtgggttcaagtccctccaccccc
a
Spacer-1 (SEQ ID NO: 589)
cgccggaaacgcaatagccgaaaaacaaaaaacaaaaaaa
CVB3 IRES (SEQ ID NO: 590)
ttaaaacagcctgtgggttgatcccacccacaggcccattgggcgctagcactctggtatcacggtacctttgtgcgcctgttttataccccct
cccccaactgtaacttagaagtaacacacaccgatcaacagtcagcgtggcacaccagccacgttttgatcaagcacttctgttaccccgga
ctgagtatcaatagactgctcacgcggttgaaggagaaagcgttcgttatccggccaactacttcgaaaaacctagtaacaccgtggaagtt
gcagagtgtttcgctcagcactaccccagtgtagatcaggtcgatgagtcaccgcattccccacgggcgaccgtggcggtggctgcgttgg
cggcctgcccatggggaaacccatgggacgctctaatacagacatggtgcgaagagtctattgagctagttggtagtcctccggcccctga
atgcggctaatcctaactgcggagcacacaccctcaagccagagggcagtgtgtcgtaacgggcaactctgcagcggaaccgactacttt
gggtgtccgtgtttcattttattcctatactggctgcttatggtgacaattgagagatcgttaccatatagctattggattggccatccggtgac
taatagagctattatatatccctttgttgggtttataccacttagcttgaaagaggttaaaacattacaattcattgttaagttgaatacagcaa
a
Spacer-2 (SEQ ID NO: 591)
aaaaaacaaaaaacaaaacggctattatgcgttaccggcg
Anabaena intron-2 (SEQ ID NO: 592)
agacgctacggacttaaataattgagccttaaagaagaaattctttaagtggatgctctcaaactcagggaaacctaaatctagttatagacaa
ggcaatcctgagccaagccgaagtagtaattagtaag
Homology arm-2 (SEQ ID NO: 593)
accagtggacaatcgacggataacagcatatctag
45A (SEQ ID NO: 594)
aaaaaaaaaaaaaaaaaaaaaaaaaaaaaaaaaaaaaaaaaaaaa
70A (SEQ ID NO: 595)
aaaaaaaaaaaaaaaaaaaaaaaaaaaaaaaaaaaaaaaaaaaaaaaaaaaaaaaaaaaaaaaaaaaaaa
150A (SEQ ID NO: 596)
aaaaaaaaaaaaaaaaaaaaaaaaaaaaaaaaaaaaaaaaaaaaaaaaaaaaaaaaaaaaaaaaaaaaaaaaaaaaaaaaaaaaaaa
aaaaaaaaaaaaaaaaaaaaaaaaaaaaaaaaaaaaaaaaaaaaaaaaaaaaaaaa???????
3′UTR (SEQ ID NO: 597)
agctcgctttcttgctgtccaatttctattaaaggttcctttgttccctaagtccaactactaaactgggggatattatgaagggccttgagcat
ctggattctgcctaataaaaaacatttattttcattgctgcgtcgagagctcgctttcttgctgtccaatttctattaaaggttcctttgttccc
taagtccaactactaaactgggggatattatgaagggccttgagcatctggattctgcctaataaaaaacatttattttcattgctgcctcgacg
aattc
anti-MSLN scFv in anti-MSLN M12.BBZ CAR (SEQ ID NO: 598)
AIRLTQSPSLLSASVGDRVTVTCRASQGGGNYLAWYQQKPGKAPKLLIYGASKLQSGVP
SRFSGSGSGTEFTLTISSLQPEDFATYYCQQLNSYPVTFGQGTKVDIKGGGGSGGGGSGG
GGSEVQLVESGAEVKKPGASVKVSCKASGYTFTTYYIHWVRQAPGQGLEWMGIINPSS
GSTTYTQKFQGRVTMTRDTSTSTVYIELSGLRSEDTAVYYCARGETLRGYFDYWGQGT
LVTVSS
anti-MSLN M12.BBZ CAR without a signal peptide (SEQ ID NO: 599)
AIRLTQSPSLLSASVGDRVTVTCRASQGGGNYLAWYQQKPGKAPKLLIYGASKLQSGVP
SRFSGSGSGTEFTLTISSLQPEDFATYYCQQLNSYPVTFGQGTKVDIKGGGGSGGGGSGG
GGSEVQLVESGAEVKKPGASVKVSCKASGYTFTTYYIHWVRQAPGQGLEWMGIINPSS
GSTTYTQKFQGRVTMTRDTSTSTVYIELSGLRSEDTAVYYCARGETLRGYFDYWGQGT
LVTVSSTTTPAPRPPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEEEGGCELRVKF
SRSADAPAYKQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPR

### REFERENCES

1. R. Alexander Wesselhoeft, Piotr S. Kowalski & Daniel G. Anderson, Engineering circular RNA for potent and stable translation in eukaryotic cells, Nature Communications, 2629 (2018).

### Example 2

### 1. In Vitro Cytotoxicity Assay of Anti-BCMA or Anti-CD19 CAR-T Cells

1.1 Prepare CAR-T cells comprising anti-BCMA CAR linear mRNA, anti-BCMA CAR circRNA, anti-CD19 CAR linear mRNA and anti-CD19 CAR circRNA, respectively. Specifically, M12.BBZ in pDA-M12.BBZ, pCA-M12.BBZ, and pCA70-M12.BBZ CAR plasmid is replaced with BCMA CAR-31 or FMC63 CAR, generating pDA-BCMA CAR-31, pCA-BCMA CAR-31, and pCA70-BCMA CAR-31. M12.BBZ in pDA-M12.BBZ, pCA-M12.BBZ, and pCA70-M12.BBZ CAR plasmid is replaced with FMC63 CAR, generating pDA-FMC63 CAR, pCA-FMC63 CAR, and pCA70-FMC63 CAR. These plasmids are transcribed in vitro and then purified as described in Example 1 to obtain BCMA CAR-31 linear mRNA, BCMA CAR-31 circRNA, BCMA CAR-31 circRNA-70A, FMC63 CAR linear mRNA, FMC63 CAR circRNA and FMC63 CAR circRNA-70A, which are then transferred to T cell by electroporation respectively to obtain anti-BCMA CAR-T and anti-CD19 CAR-T cells, wherein linear mRNA is derived from pDA-CAR plasmid, circRNA is derived from pCA-CAR plasmid and circRNA-70A is derived form pCA70-CAR plasmid. BCMA CAR-31 is a CAR comprising GM-CSF signal peptide, BCMA31 scFv (SEQ ID NO: 244), CD8 hinge domain, CD8 transmembrane (TM) domain, 4-1BB costimulatory domain and CD3ζ domain. The amino acid sequence of BCMA CAR-31 is as set forth in SEQ ID NO: 256 and the nucleic acid sequence of BCMA CAR-31 is as set forth in SEQ ID NO: 1003. FMC63 CAR is an anti-CD19 CAR commercially available, comprising CD8 signal peptide, an anti-FMC63 scFv, CD8 hinge domain, CD8 transmembrane (TM) domain, 4-1BB costimulatory domain and CD3ζ domain the amino acid sequence of which is as set forth in SEQ ID NO: 997 and the nucleic acid sequence of which is as set forth in SEQ ID NO: 998.
1.2 12 hours before coculture, seed RPMI-8226-CBG and Raji-CBG to flat-bottomed 96-well plate with 3000 cells/100 μl/well.
1.3 About 12 to 24 hours post electroporation, count and dilute CAR-T cells to appropriate concentration, seed cells with 100 μl/well to tumor cells with E/T ratio 2:1.
1.4 Put the co-culture plate into IncuCyte S3 machine, and setup scanning parameters.
1.5 After 4 days scanning, the Total Green Object Integrated Intensity (GCU×μm²/Well) was analyzed to calculate the killing efficiency.

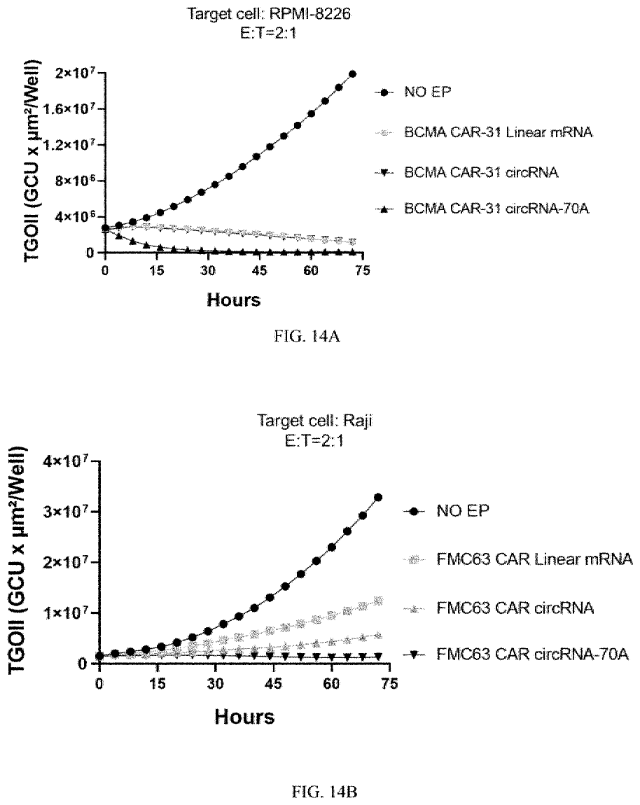

Coculture assay using these CAR-T cells with BCMA or CD19 expressing tumor cells also demonstrated the higher killing ability of the circRNA-70A CAR-T group than linear mRNA CAR-T group and circRNA CAR-T group (FIG. 14A-14B).

### 2. In Vitro Cytotoxicity Assay of LACOSTIM-Expressing M12 CART Cells, LACOSTIM-Expressing BCMA CART Cells, LACOSTIM-Expressing FMC63 CART Cells.

2.1 LACOSTIM-expressing M12 CAR-T cells are generated by electroporated with LACOSTIM and M12 CAR linear mRNA or circRNA. Specifically, M12.BBZ in pDA-M12.BBZ and pCA70-M12.BBZ CAR plasmid is replaced with A40C28 (SEQ ID NO: 600), generating pDA-A40C28 and pCA70-A40C28 plasmid. M12.BBZ linear mRNA and M12.BBZ circRNA-70A are prepared as described in Example 1. BCMA CAR-31 linear mRNA, BCMA CAR-31 circRNA-70A, FMC63 CAR linear mRNA, and FMC63 CAR circRNA-70A are prepared as described above in this Example. pDA-A40C28 and pCA70-A40C28 plasmid is transcribed in vitro and then purified as described in Example 1 to obtain A40C28 linear mRNA and A40C28 circRNA-70A.
2.2 LACOSTIM-expressing M12 CAR-T cells are generated by electroporated 5E6 T cells with g M12.BBZ linear RNA and 10 μg A40C28 linear RNA, or 10 μg M12.BBZ circRNA-70A and 10 μg A40C28 circRNA. LACOSTIM-expressing BCMA31 CAR-T cells and LACOSTIM-expressing CD19 CAR-T cells are prepared by the same method, except that M12.BBZ linear RNA and M12.BBZ circRNA-70A are replaced by BCMA CAR-31 linear mRNA and BCMA CAR-31 circRNA-70A, or FMC63 CAR linear mRNA and FMC63 CAR circRNA-70A.
2.3 12 hours before coculture, seed OVCAR3-CBG, RPMI-8226-CBG and Raji-CBG to flat-bottomed 96-well plate with 3000 cells/100 l/well.
2.4 About 12 to 24 hours post electroporation, count and dilute three groups of CAR-T cells (see the following Table 1 for details.) to appropriate concentration, seed cells with 100 ul/well to tumor cells with E/T ratio 2:1.

TABLE 1
2.5

| Group | Different electroporation components | mRNA quality |
|---|---|---|
| Group 1 | NO EP | 0 |
| | M12 CAR linear mRNA | 10 μg |
| | M12 CAR circRNA-70A | 10 μg |
| | M12 CAR + A40C28 linear mRNA | 10 μg, 10 μg |
| | M12 CAR + A40C28 circRNA-70A | 10 μg, 10 μg |
| Group 2 | NO EP | 0 |
| | BCMA CAR-31 linear mRNA | 10 μg |
| | BCMA CAR-31 circRNA-70A | 10 μg |
| | BCMA CAR-31 + A40C28 linear | 10 μg, 10 μg |
| | mRNA | |
| | BCMA CAR-31 + A40C28 circRNA- | 10 μg, 10 μg |
| | 70A | |
| Group 3 | NO EP | 0 |
| | FMC63 CAR linear mRNA | 10 μg |
| | FMC63 CAR circRNA-70A | 10 μg |
| | FMC63 CAR + A40C28 linear mRNA | 10 μg, 10 μg |
| | FMC63 CAR + A40C28 circRNA-70A | 10 μg, 10 μg |

2.6 Put the co-culture plate into IncuCyte S3 machine, and setup scanning parameters.
2.7 After 4 days scanning, the Total Green Object Integrated Intensity (GCU×μm²/Well) was analyzed to calculate the killing efficiency.

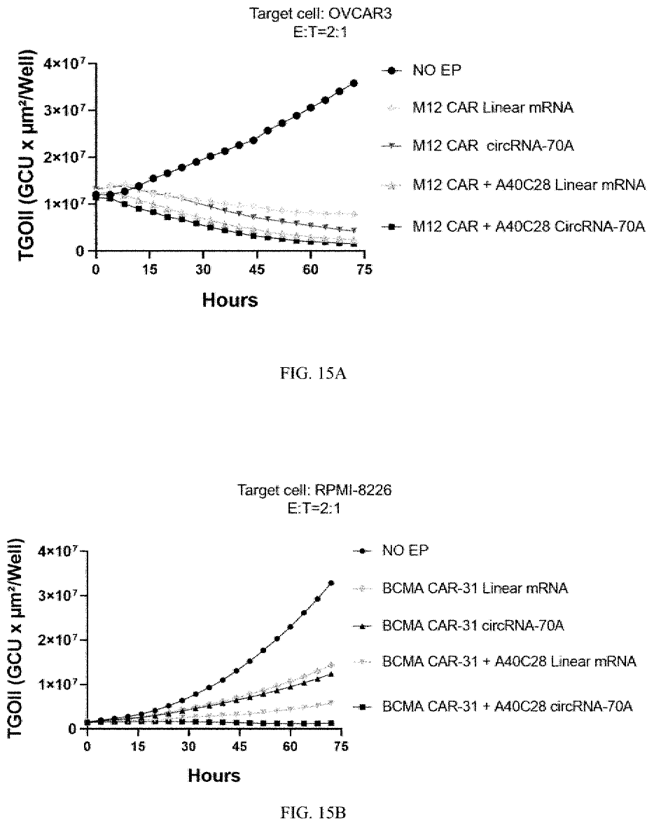

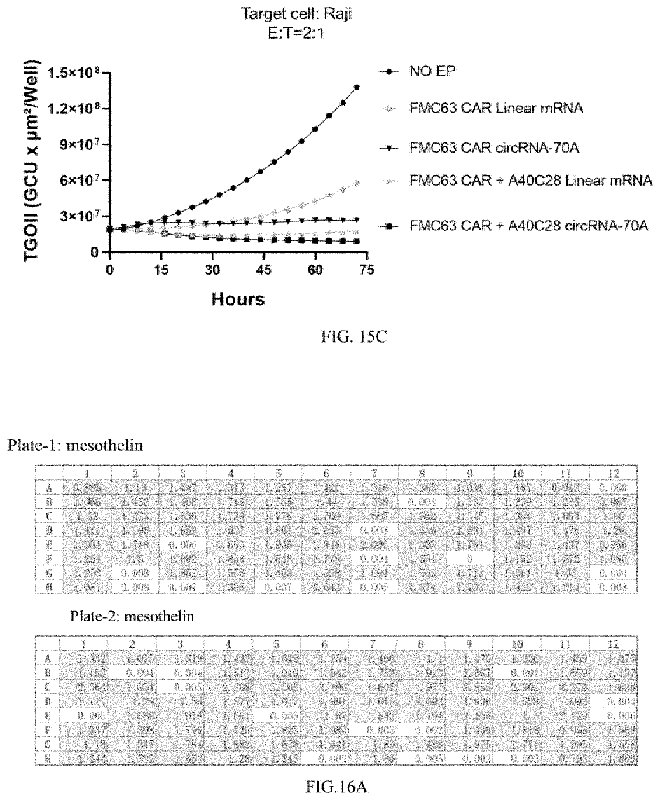

As shown in FIG. 15A-15C, coculture assay using these CAR-T cells with or without LACOSTIM-expressed demonstrated the higher killing ability of the circRNA-70A CAR-T group than linear mRNA CAR-T group, LACOSTIM-expressed CAR-T cells have higher killing ability than CAR only cells, and LACOSTIM-expressed CAR-T cells in circRNA also have the higher killing ability of linear LACOSTIM-expressed CAR-T cells.

### Example 3: Preparation of Antibodies and CARs Targeting MESO, CD123 and BCMA

### Preparation of Antibodies

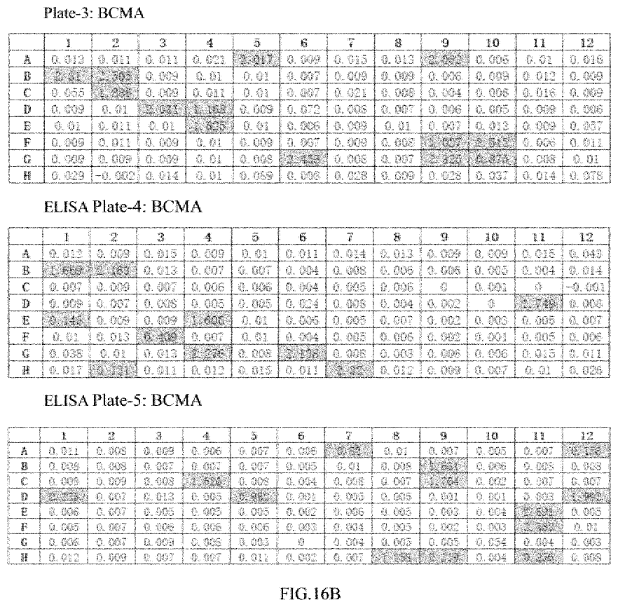

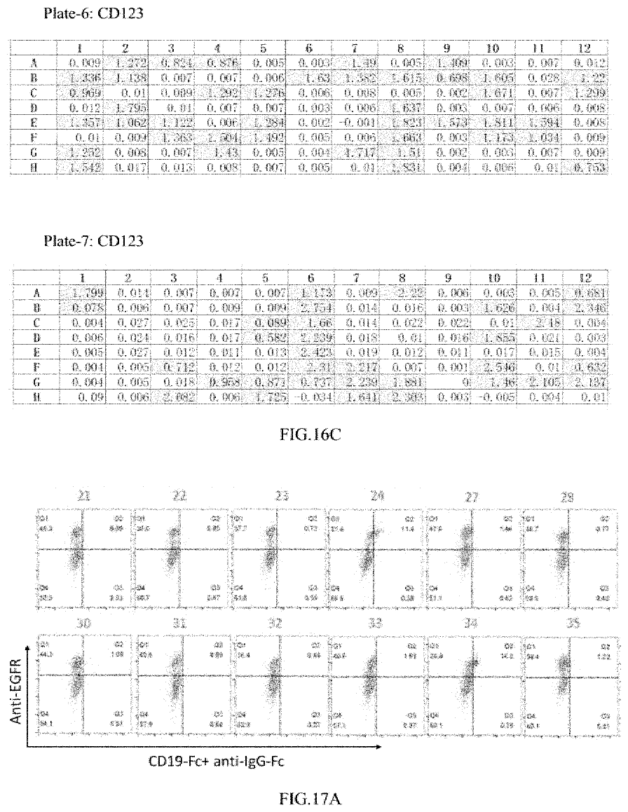

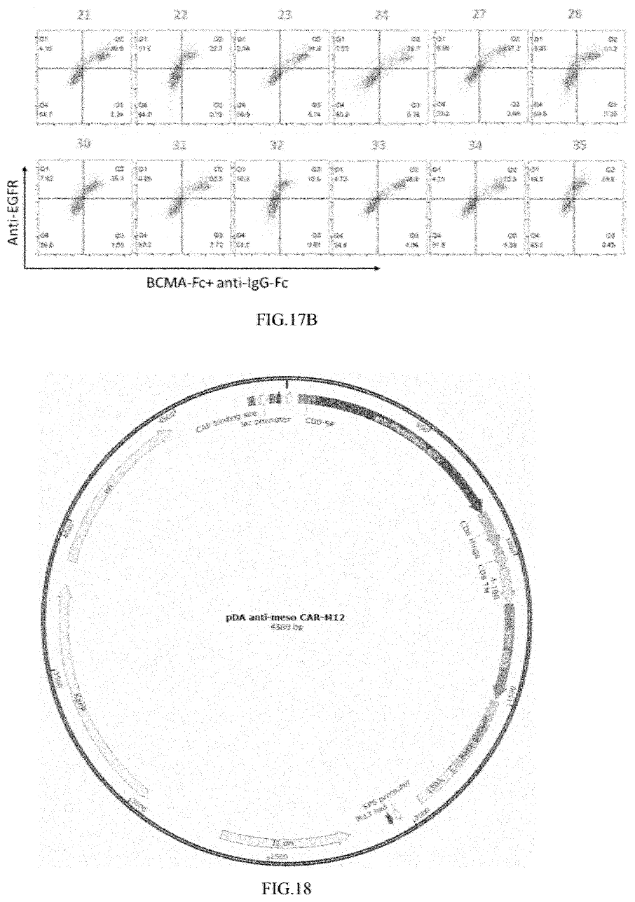

Antibodies were prepared using fully human antibody phage display library, and have been described in Patent Application No. PCT/CN2022/112724, PCT/CN2022/112728 and PCT/CN2022/112726, which are capable of specifically recognizing mesothelin (MESO) (a total of 37 antibodies, M1-M37 respectively), CD123 (a total of 35 antibodies, C1-C35 respectively), and BCMA (a total of 15 antibodies, BCMA21-BCMA35 respectively), respectively. FIG. 16A-16C provides the reads of two 96-well plates of anti-human mesothelin (or BCMA, CD123)-Fc monoclonal phage ELISA.

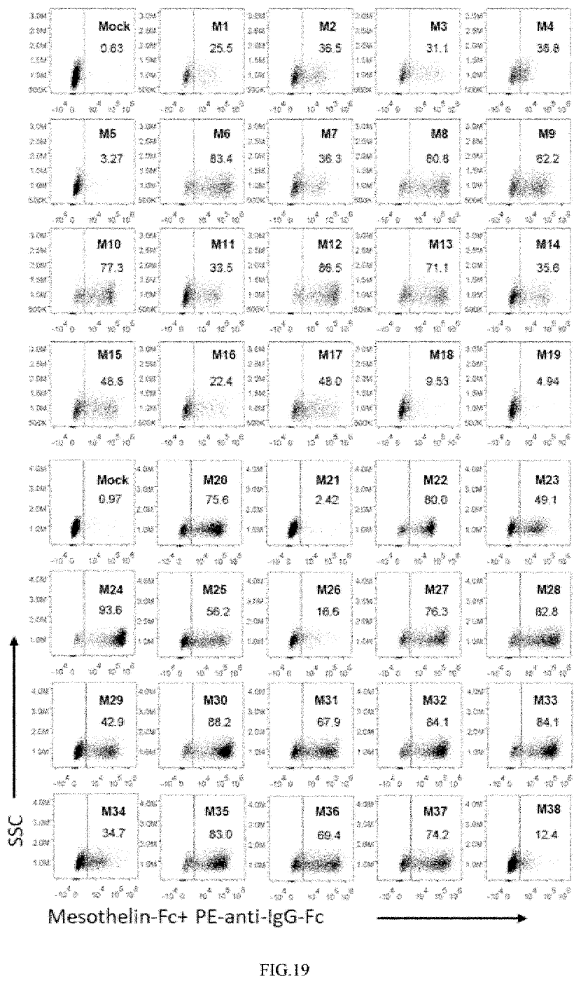

Cloning and sequence analysis: positive clones were selected according to the ELISA results and used as templates for PCR cloning of the scFv sequence. The CDR regions of scFv were analyzed through abysis website (http://abysis.org/) according to Kabat numbering scheme. Screening of functional anti-BCMA scFv(s) in T cell: anti-BCMA scFv(s) were constructed into a bicistronic lentiviral CAR expression vector, which contained an IRES-truncated EGFR (tEGFR) expressing cassette. Lentivirus was generated by transient transfection in 293T cells, then purified and concentrated by ultra-centrifuge. T cells were transduced with CAR lentivirus to generate CAR-T cells, and cultured for another 10 days. 10 days after lentivirus-transduction, CAR-T cells were collected and stained with 5 μg/ml CD19-Fc protein (Ctrl Fc protein) or BCMA-Fc recombinant protein at 4° C. for 30 min. After washing, the CAR-T cells were stained with anti-human IgG Fc and anti-EGFR mAb. Sample was analyzed by flow cytometry. As shown, T cells expressing CARs comprising the following anti-BCMA scFv(s) showed binding to BCMA-Fc (FIG. 17B) and were selected for further studies: BCMA21 (SEQ ID NO: 237), BCMA22 (SEQ ID NO: 238), BCMA23 (SEQ ID NO: 239), BCMA24 (SEQ ID NO: 240), BCMA27 (SEQ ID NO: 241), BCMA28 (SEQ ID NO: 242), BCMA30 (SEQ ID NO: 243), BCMA31 (SEQ ID NO: 244), BCMA32 (SEQ ID NO: 245), BCMA33 (SEQ ID NO: 246), BCMA34 (SEQ ID NO: 247), and BCMA35 (SEQ ID NO: 248).

### Preparation of CARs

Vectors for generating mRNA of different target CAR were constructed. First, the scFv sequences of the above antibodies with a CD8 signal peptide at the N-terminus and CAR fragment (from hinge domain to CD3-zeta domain, comprising CD8 hinge domain, CD8 transmembrane domain, 4-1BB costimulatory domain and CD3ζ domain.) were amplified by PCR and cloned into pDA vector (Xba1/Sal1). FIG. 18 provides the schematic representation of pDA-CAR vector used for CAR mRNA generation as an example. Second, different CAR mRNA was prepared by in vitro transcription (IVT). The pDA-CAR plasmid was linearized by digestion with Spe1 enzyme and purified by PCR Cleanup kit. After the DNA concentration was measured by nanodrop and checked by running agarose DNA gel, IVT was performed following the protocol of manufacturer (Thermofisher, Cat No: AM13455). The concentration of RNA product was measured by nanodrop and checked by running PAGE gel.

### Example 4: Preparation and Characterization of CARTs

CAR mRNA of different targets prepared in Example 3 was introduced into A549 tumor cells and T cells (CART cells) by electroporation with the following procedures: A549 tumor cells and T cells were collected and washed with Opti-MEM medium for 3 times. The cell pellets were resuspended with Opti-MEM medium, and the cell concentration was adjusted to 1×10e7/ml. 10 μg RNA was aliquoted to 1.5 ml EP tube, added with 100 μl T cells or A549 cells, and mixed well. 100 μl cells mixed with RNA were added to the BTX electroporation cup, tapped to avoid bubble. Electroporation was performed using BTX machine at the following parameters: For T cells: 500 voltage, 0.7 ms; for A549 tumor cell: 300 voltage, 0.5 ms. The cells were then transferred to pre-warmed culture medium and culture at 37° C.

### (1) Mesothelin

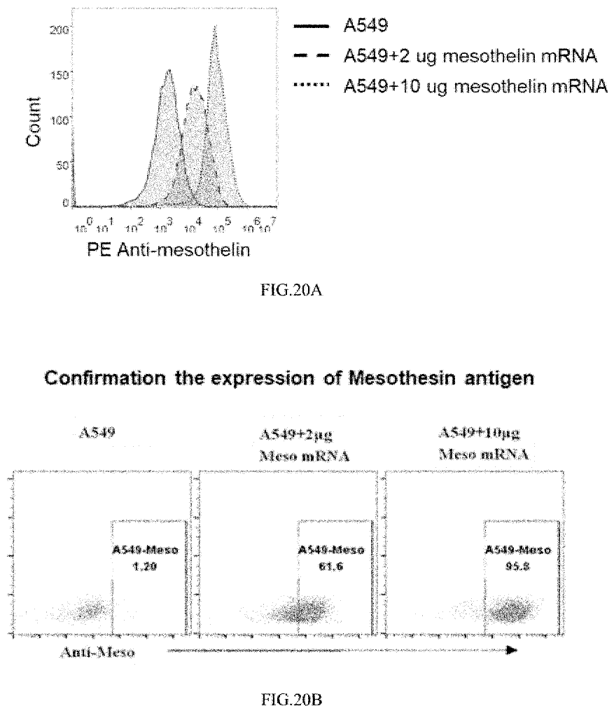

Binding of CART cells to different targets-Fc recombinant protein was measured by FACS staining. As shown in FIG. 19, anti-mesothelin scFv-M1, -M2, -M3, -M6, -M7, -M8, -M9, -M10, -M11, -M12, -M13, -M14, -M15, -M16, -M17, -M20, -M22, -M23, -M24, -M25, -M27, -M28, -M29, -M30, -M31, -M32, -M33, -M34, -M35, -M36 and -M37, bound to mesothelin-Fc recombinant protein. T cell without CAR molecule served as control (“Mock”). As shown in FIG. 20A-20B, the ectopic expression level of mesothelin correlated with the amount of mesothelin mRNA that was introduced into A549 cells via electroporation.

The cytotoxicity of the mesothelin CART cells against tumor cells was measured in in vitro cytotoxicity assay. EGFP-expressing tumor cell lines or EGFP-A549 cells that were electroporated with different amount of tumor antigen were seeded on flat-bottomed 96-well plate at 3000 cells/100 μl/well. CART cells were diluted to appropriate concentration, seeded at 100 l/well with tumor cells at different E/T ratios, such as 10:1, 3:1, 1:1. The co-culture plates were placed into IncuCyte S3 machine, and scanning parameters were set. After 3 days of scanning, the Total Green Object Integrated Intensity (GCU×μm² μl/well) was analyzed to calculate the killing efficiency.

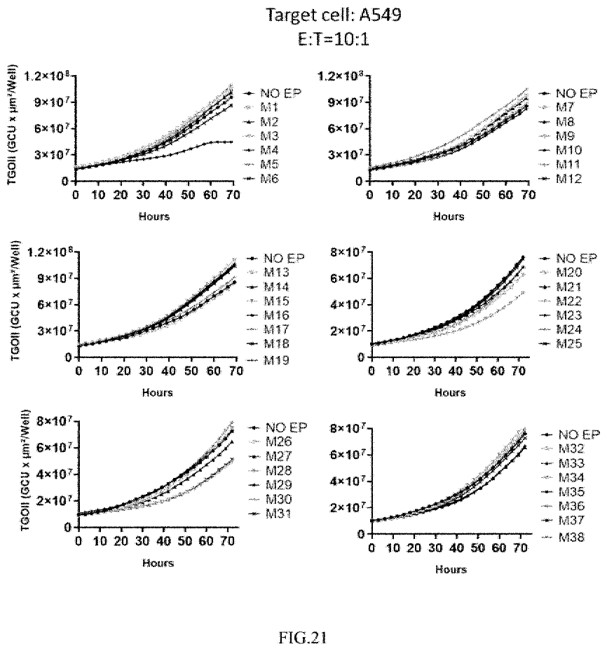

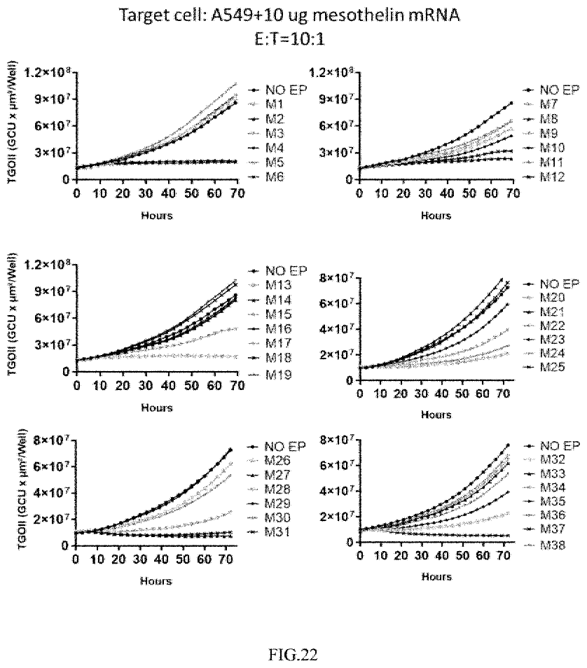

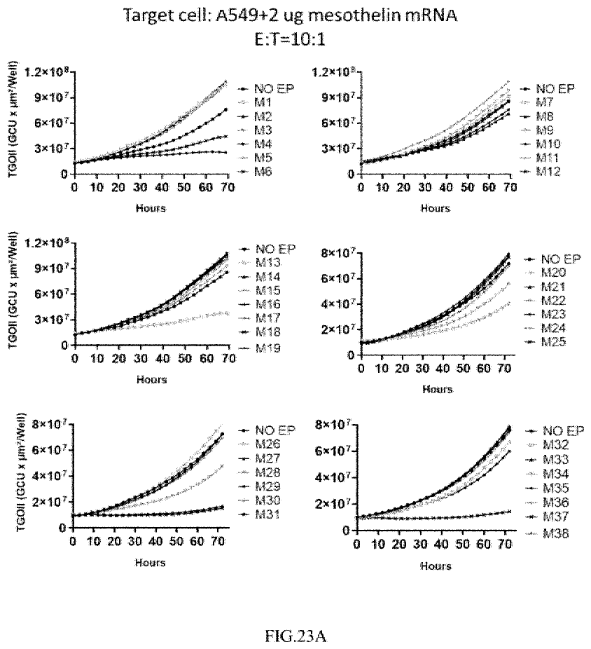

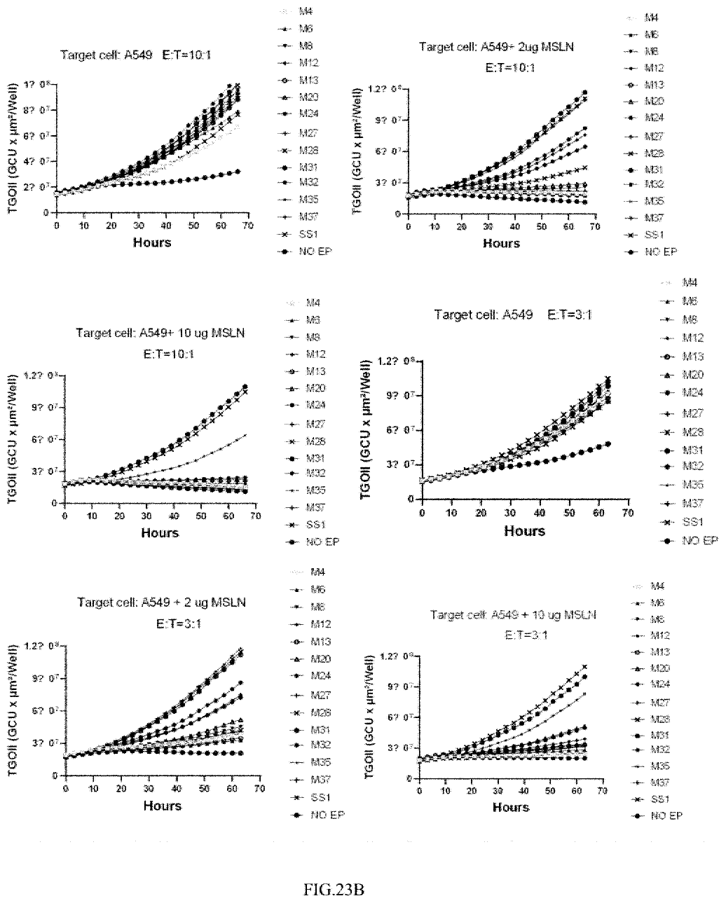

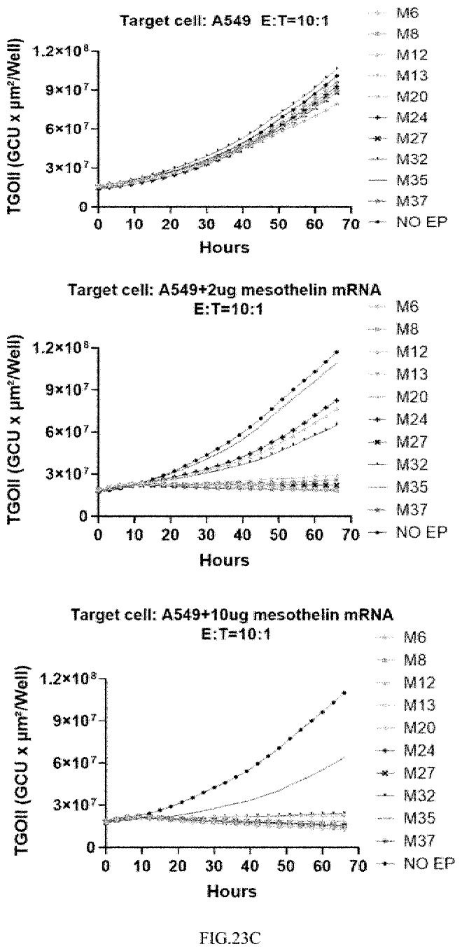

A549 cells express mesothelin at low level. As shown in FIG. 21, CART cells expressing anti-mesothelin scFv-M4, -M22, -M28 and -M31 effectively impeded the growth of A549 cells, indicating that these scFv-based CART cells had comparably high cytotoxicity against tumor cells. As shown in FIG. 22, CART cells expressing anti-mesothelin scFv-M4, -M6, -M7, -M8, -M9, -M10, -M11, -M12, -M13, -M15, -M20, -M22, -M23, -M24, -M27, -M28, -M31, -M32, -M35, -M37 and -M38 CART cells showed effectively killing effect toward the mesothelin overexpressing A549 tumor cells (electroporated with 10 μg mesothelin mRNA). As shown in FIG. 23A, CART cells expressing anti-mesothelin scFv-M4, -M6, -M13, -M20, -M27, -M31, and -M37 CART maintained strong killing effect to A549 tumor cells with less ectopic expression of mesothelin (electroporated with 2 μg mesothelin mRNA). CART cells expressing anti-mesothelin scFv-M7, -M8, -M9, -M10, -M11, -M12, -M15, -M23, -M24, -M32, -M35, and -M38, which selectively showed high cytotoxicity toward tumor cells with high mesothelin expression but not those with low mesothelin expression had superior safety, as mesothelin is expressed in certain normal tissues.

A separate experiment was conducted using ss1 CAR transduced T cells as a control. T cells that were transduced with M12 CARs displayed best specificity and effectivity than ss1 CART cells among all the CARs tested, (FIG. 23B). T cell with CAR molecule derived from the single chain anti-mesothelin monoclonal antibody SS1 (scFv, published in Chowdhury, et al., 1998, Proc Natl Acad Sci USA 95: 669-674) served as control (“SS1”). and the construction of ss1 CAR comprises ss1.BBZ, CD8 hinge, CD8 transmembrane domain, 4-1BB co-stimulatory domain and CD3-zeta signaling domain.

FIG. 23C. shows the killing curves of different mRNA-based anti-mesothelin CART cells, with A549-GFP tumor cells electroporated with 0 (upper panel), 2 μg (middle panel) or 10 μg (lower panel) mesothelin mRNA being the target cells (E/T ratio=10:1). As shown, that anti-mesothelin scFv-M12, -M24 and -M32 CART cells had moderate killing effect toward the A549 tumor cells with low mesothelin expression (2 μg group), but strong killing effect toward A549 tumor cells with high mesothelin expression (10 μg group). The results indicated that these CART cells would specifically target tumor cells with high mesothelin expression, and spare the normal tissues with low mesothelin expression.

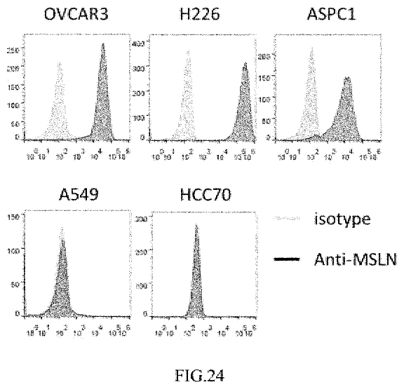

FIG. 24 shows the FACS staining of OVCAR3 (human ovarian cancer cells), H226 (human lung carcinoma cells), ASPC1 (human pancreatic tumor cells), A549 (human lung cancer cells) and HCC70 (human breast cancer cells) with isotype control and anti-mesothelin mAb. As shown, certain cancer cells, including OVCAR3, H226 and ASPC1, express mesothelin at high level; A549 express mesothelin at low level, and HCC70 does not express mesothelin.

### (2) BCMA

we constructed 12 different anti-BCMA CARs using the anti-BCMA scFv(s) described above. Three other CART products were tested in parallel, including NBC10 (Novartis AG and University of Pennsylvania, BMCA10.BBz), FHVH33 (National Institutes of Health, US), and B38M (Nanjing Legend Biotech). All tested CARs had a 41BBz coactivation domain.

TABLE 2
BCMA CARTs, CAR %, and Expression Levels

| CART | scFv | CAR % | MFI |
|---|---|---|---|
| 1 | NBC10 | 22% | 1.2E+05 |
| 2 | FHVH33 | 84% | 3.2E+05 |
| 3 | BCMA21 | 31% | 1.6E+05 |
| 4 | BCMA22 | 12% | 7.5E+04 |
| 5 | BCMA23 | 18% | 2.2E+05 |
| 6 | BCMA24 | 23% | 9.4E+04 |
| 7 | BCMA27 | 27% | 1.7E+05 |
| 8 | BCMA28 | 29% | 6.5E+04 |
| 9 | BCMA30 | 25% | 4.6E+04 |
| 10 | BCMA31 | 37% | 2.5E+05 |
| 11 | BCMA32 | 19% | 5.6E+04 |
| 12 | BCMA33 | 27% | 2.6E+05 |
| 13 | BCMA34 | 16% | 9.5E+04 |
| 14 | BCMA35 | 18% | 1.1E+05 |
| 15 | B38M | 51% | 5.8E+05 |
| 16 | NTD | | |

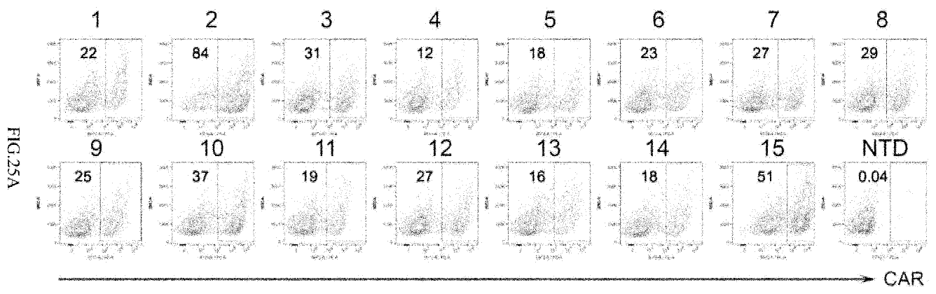

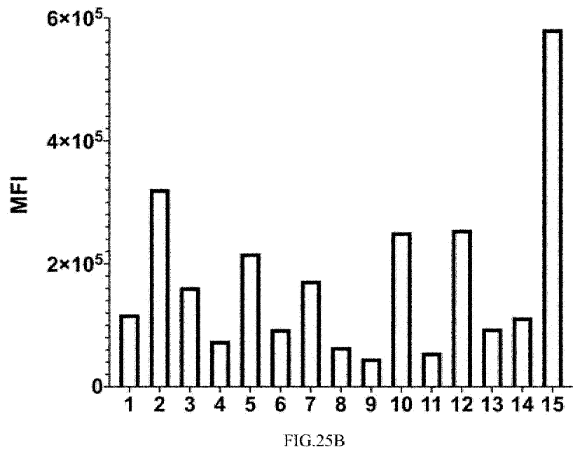

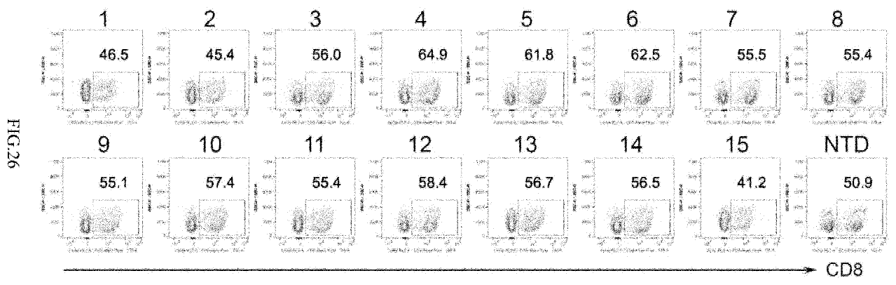

T cells were transduced by lentiviral vectors to express different BCMA CARs. Table 2 above shows the CART cells used in the studies disclosed herein, the percentage of the CAR-expressing cells, and their respective expression levels. FIGS. 25A and 25B show the frequencies of CAR+ T cells and their expression levels (Mean Fluorescence Intensity; “MFI”), respectively. Among the twelve scFv we generated, BCMA31 (#10; SEQ ID NO:256) and BCMA33 (#12; SEQ ID NO:258) were expressed at higher levels than the rest. FIG. 26 shows comparable frequencies of CAR+CD8 cells among tested CARTs. FIG. 27 shows the phenotypes of CART cells. The frequencies of naive T cell population (CD45RO−; CCR7+) in BCMA27 (#7), BCMA31 (#10) and BCMA33 (#12) T cells were higher than those in other samples, indicating these T cells were less differentiated.

### (3) CD123

CD123 CAR mRNA was introduced into T cells by electroporation with the following procedures: T cells were collected and washed with Opti-MEM medium, and resuspended with Opti-MEM medium at 1×10e7/ml; 10 μg RNA was aliquoted with 100 μl T cells, mixed well for electroporation at the following parameters (BTX machine): 500 voltage, 0.7 ms; the cells were then transferred to pre-warmed culture medium and culture at 37° C.

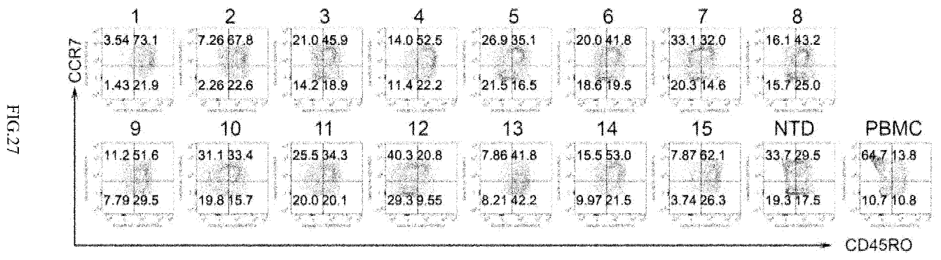

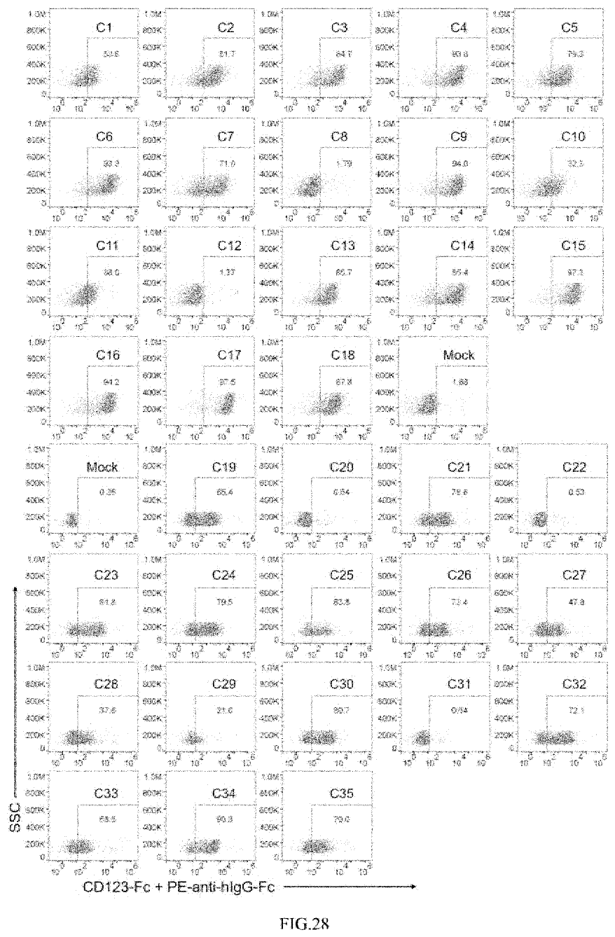

Binding of CD123 CART cells to CD123-Fc recombinant protein was measured by FACS staining. As shown in FIG. 28, T cells expressing CARs having anti-CD123 scFv-C1, -C2, -C3, -C4, -C5, -C6, -C7, -C9, -C10, -C11, -C13, -C14, -C15, -C16, -C17, -C18, -C19, -C21, -C23, -C24, -C25, -C26, -C27, -C28, -C29, -C30, -C32, -C33, -C34 and -C35 were able to bind to CD123-Fc recombinant protein. Mock was control T cell without CAR molecule.

A549 tumor cells were electroporated with different amount of CD123 mRNA. The electroporation procedure was the same as described above for T cells, except that a setting of 300 voltage, 0.5 ms was used. Expression of CD123 in A549 tumor cells was measured by FACS staining of the A549 cells electroporated with different amount of CD123 mRNA with isotype or anti-CD123 antibody. As shown in FIG. 29, A549 cells weakly expressed endogenous CD123, and the ectopic expression level of CD123 correlated with the amount of CD123 mRNA that was electroporated into A549 cells.

The cytotoxicity of the CD123 CART cells against tumor cells was measured in in vitro cytotoxicity assay. EGFP-expressing tumor cells or EGFP-A549 cells that were electroporated with different amount of tumor antigen were seeded on flat-bottomed 96-well plate at 3000 cells/100 ul/well; CART cells were diluted to appropriate concentration and seeded with 100 ul/well tumor cells at different E/T ratios, such as 10:1, 3:1, 1:1; the co-culture plates were then placed in IncuCyte S3 machine, and scanning parameters were set. After 3 days scanning, the Total Green Object Integrated Intensity (GCU×μm²/Well) was analyzed to calculate the killing efficiency.

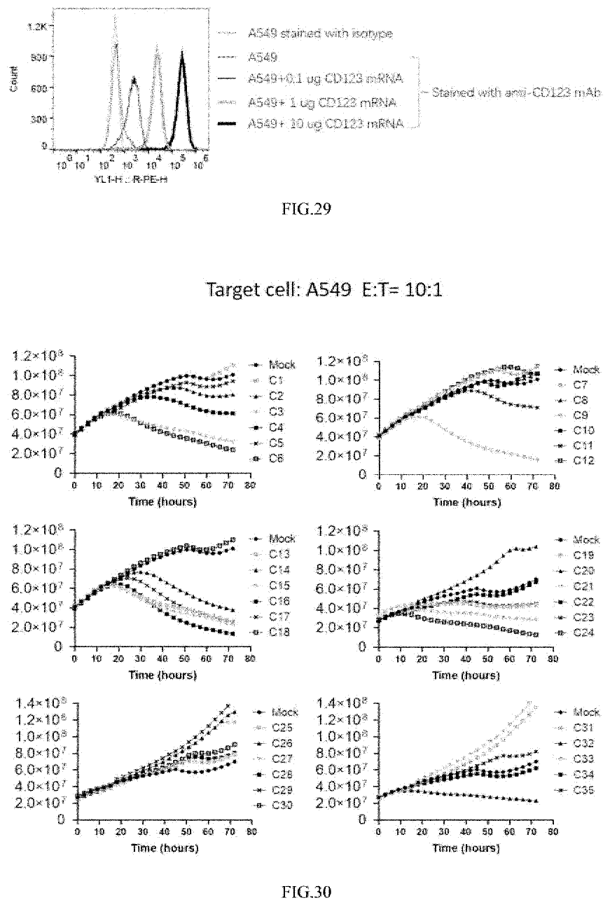

FIG. 30 and FIG. 31 show the killing curves of different mRNA-based anti-CD123 CART cells against A549-GFP tumor cells at E/T ratio of 10:1 (FIG. 30) or 3:1 (FIG. 31). As shown, CART cells expressing anti-CD123 scFv-C2, -C3, -C4, -C6, -C9, -C11, -C13, -C14, -C15, -C16, -C17, -C19, -C21, -C23, -C24 and -C32 effectively arrested the growth and even eliminated A549 cells, despite that A549 cells expressed low level endogenous CD123, indicating that these scFv-based CART cells had comparably high cytotoxicity against tumor cells.

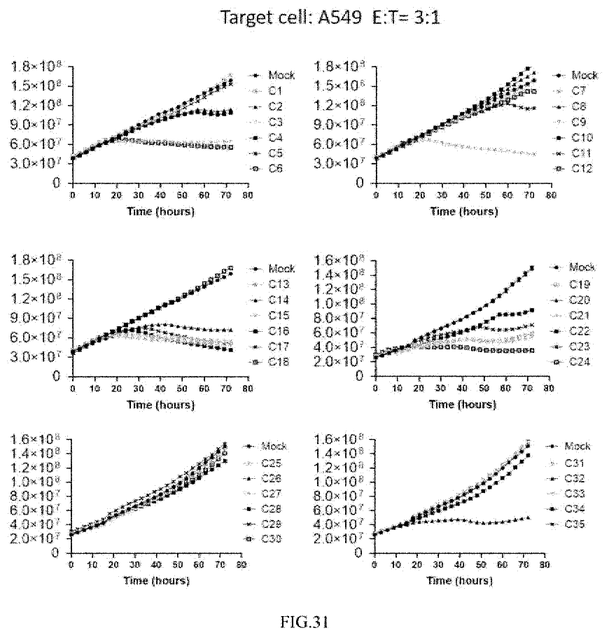

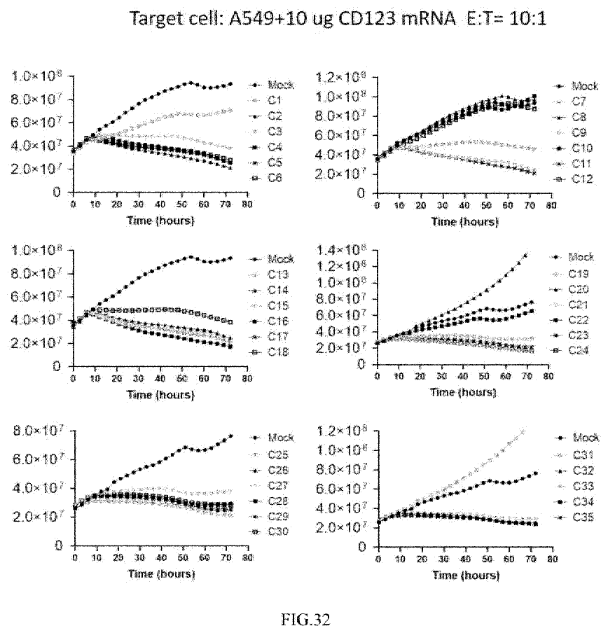

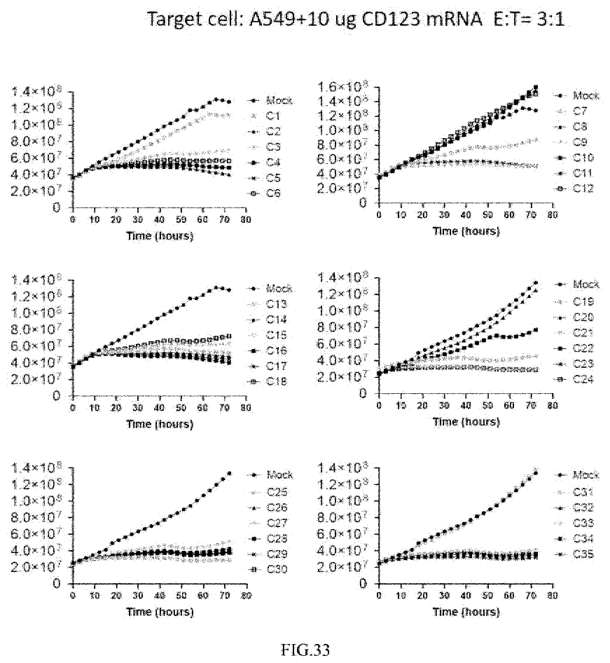

FIG. 32 and FIG. 33 show the killing curves of different mRNA-based anti-CD123 CART cells against A549-GFP tumor cells that expressed exogenous CD123 (electroporated with 10 μg CD123 mRNA) at E/T ratio of 10:1 (FIG. 32) or 3:1 (FIG. 33). As shown, CART cells expressing anti-CD123 scFv-C1, -C2, -C3, -C4, -C5, -C6, -C7, -C9, -C11, -C13, -C14, -C15, -C16, -C17, -C18, -C19, -C21, -C23, -C24, -C25, -C26, -C27, -C28, -C29, -C30, -C32, -C33, -C34 and -C35 CART cells effectively arrested the growth and even reduced the number of the CD123 expressing-A549 tumor cells, confirming their abilities to kill CD123 expressing tumor cells.

The sequences of the screened antibodies and CARs in Examples 1-2 are shown in Tables 3 and 4.

TABLE 3
The CDR regions of scFv (Kabat)

| Antibody | VL CDR1 | VL CDR2 | VL CDR3 |
|---|---|---|---|
| M6 | RASQSIGNSLA | DVSNRAT | QHRYSWPLT |
| | (SEQ ID NO: 1) | (SEQ ID NO: 16) | (SEQ ID NO: 30) |
| M7 | RASQAISSALA | DASTLES | QQADSFPLT |
| | (SEQ ID NO: 2) | (SEQ ID NO: 17) | (SEQ ID NO: 31) |
| M8 | RASQSISSSLN | AASSLQS | QQSYSTPLT |
| | (SEQ ID NO: 3) | (SEQ ID NO: 18) | (SEQ ID NO: 32) |
| M10 | RASQSISSWLA | KASSLES | QQYYSYPLT |
| | (SEQ ID NO: 4) | (SEQ ID NO: 19) | (SEQ ID NO: 33) |
| M12 | RASQGGGNYLA | GASKLOS | QQLNSYPVT |
| | (SEQ ID NO: 5) | (SEQ ID NO: 20) | (SEQ ID NO: 34) |
| M13 | RASQGISNSLA | AASRLES | QQYYSTPFT |
| | (SEQ ID NO: 6) | (SEQ ID NO: 21) | (SEQ ID NO: 35) |
| M15 | RASQGVNSALA | DASSLES | QQFSSYPLT |
| | (SEQ ID NO: 7) | (SEQ ID NO: 22) | (SEQ ID NO: 36) |
| M20 | RASQGISSAVA | YASSLES | QQFNSYPLT |
| | (SEQ ID NO: 8) | (SEQ ID NO: 23) | (SEQ ID NO: 37) |
| M22 | TGTRRDIGGYEYVS | SVNNRPS | SSYSSRDTLVL |
| | (SEQ ID NO: 9) | (SEQ ID NO: 24) | (SEQ ID NO: 38) |
| M24 | SGSSSNIGSNYVY | MNNQRPS | AARDDSLSGYVT |
| | (SEQ ID NO: 10) | (SEQ ID NO: 25) | (SEQ ID NO: 39) |
| M27 | RASQPIVASHLA | GASTRAA | QQYGISPF |
| | (SEQ ID NO: 11) | (SEQ ID NO: 26) | (SEQ ID NO: 40) |
| M28 | SGTSSDVGGYNFVS | EVSKRPS | SSYAGRNNPYL |
| | (SEQ ID NO: 12) | (SEQ ID NO: 27) | (SEQ ID NO: 41) |
| M31 | TGTSSDIGGYNSVS | GVSRRPS | SSYGGSNNLL |
| | (SEQ ID NO: 13) | (SEQ ID NO: 28) | (SEQ ID NO: 42) |
| M32 | QASEDINNSLN | DASDLET | QQLNSYPLT |
| | (SEQ ID NO: 14) | (SEQ ID NO: 29) | (SEQ ID NO: 43) |
| M37 | RASQGISNYLA | AASSLOS | QQYSSYPIT |
| | (SEQ ID NO: 15) | (SEQ ID NO: 18) | (SEQ ID NO: 44) |
| Antibody | VH CDR1 | VH CDR2 | VH CDR3 |
| M6 | DYYMS | YISSSGSTIYYADSVKG | DGYRTHNWFDP |
| | (SEQ ID NO: 45) | (SEQ ID NO: 58) | (SEQ ID NO: 71) |
| M7 | AYWIA | IIYPGDSRVIYSPYFQG | FGGPKFATNWFDI |
| | (SEQ ID NO: 46) | (SEQ ID NO: 59) | (SEQ ID NO: 72) |
| M8 | GYAMY | AITTNGGSTNYADSVKG | GAPGYRGYYMDV |
| | (SEQ ID NO: 47) | (SEQ ID NO: 60) | (SEQ ID NO: 73) |
| M10 | SYAMS | AISGSGGSTYYADSVKG | RGSSWYFDY |
| | (SEQ ID NO: 48) | (SEQ ID NO: 61) | (SEQ ID NO: 74) |
| M12 | TYYIH | IINPSSGSTTYTQKFQG | GETLRGYFDY |
| | (SEQ ID NO: 49) | (SEQ ID NO: 62) | (SEQ ID NO: 75) |
| M13 | ANTIN | RIIPSLNIRDYAQEFQG | DPGSTWSPNQFFPH |
| | (SEQ ID NO: 50) | (SEQ ID NO: 63) | (SEQ ID NO: 76) |
| M15 | SYYMH | IINPSGGSTSYAQKFQG | AQRGGSVYFDY |
| | (SEQ ID NO: 51) | (SEQ ID NO: 64) | (SEQ ID NO: 77) |
| M20 | DSWIA | IIFPGDSNPIYSPSFQG | HAAWGAGWFDP |
| | (SEQ ID NO: 52) | (SEQ ID NO: 65) | (SEQ ID NO: 78) |
| M22 | SYAIS | GIIPIFGTANYAQKFQG | GRSGSYGLY |
| | (SEQ ID NO: 53) | (SEQ ID NO: 66) | (SEQ ID NO: 79) |
| M24 | SYAMS | AISGSGGSTYYADSVKG | GVATFDY |
| | (SEQ ID NO: 48) | (SEQ ID NO: 61) | (SEQ ID NO: 80) |
| M27 | DYWIG | WITPNNGNTNYAPKFQG | RGRNSSGYLYYYSMDV |
| | (SEQ ID NO: 54) | (SEQ ID NO: 67) | (SEQ ID NO: 81) |
| M28 | SYAIS | GIIPIFGTANYAQKFQG | DLGGYSYGHGLDY |
| | (SEQ ID NO: 53) | (SEQ ID NO: 66) | (SEQ ID NO: 82) |
| M31 | SSWMA | NIKQDGSSQYYVDSVKG | DIWYSIDY |
| | (SEQ ID NO: 55) | (SEQ ID NO: 68) | (SEQ ID NO: 83) |
| M32 | GYYLH | IINPSGGRTSMAQKFQG | ADNWNAGSMDV |
| | (SEQ ID NO: 56) | (SEQ ID NO: 69) | (SEQ ID NO: 84) |
| M37 | PYYWT | YIHYSGRTNYNPSLES | VGDPGLFDY |
| | (SEQ ID NO: 57) | (SEQ ID NO: 70) | (SEQ ID NO: 85) |
| Antibody | VL CDR1 | VL CDR2 | VL CDR3 |
| BCMA21 | RASQSVSSNFLA | GASNRAT | QHYDGSPPMYT |
| | SEQ ID NO: 146 | SEQ ID NO: 156 | SEQ ID NO: 167 |
| BCMA22 | TGTSSDVGGYNYVS | EVTNRPS | ISYTSSSTLDYV |
| | SEQ ID NO: 147 | SEQ ID NO: 157 | SEQ ID NO: 168 |
| BCMA23 | SGSSPNIGGNSVN | TNNQRPS | AAWDDSLNGVV |
| | SEQ ID NO: 148 | SEQ ID NO: 158 | SEQ ID NO: 169 |
| BCMA24 | SGSSSNIGSYSVN | SNNQRPS | AAWDDSLNGVV |
| | SEQ ID NO: 149 | SEQ ID NO: 159 | SEQ ID NO: 169 |
| BCMA27 | TGSRSNVGSYNDVS | DVDKRPA | SSYGGTYSLFV |
| | SEQ ID NO: 150 | SEQ ID NO: 160 | SEQ ID NO: 170 |
| BCMA28 | SGSSSNIGQNAVN | YNDLVSS | ATWDDSLNGVV |
| | SEQ ID NO: 151 | SEQ ID NO: 161 | SEQ ID NO: 171 |
| BCMA30 | RASQGISSYLA | AASTLQS | QQLNSYPPWT |
| | SEQ ID NO: 152 | SEQ ID NO: 162 | SEQ ID NO: 172 |
| BCMA31 | TGTSSDVGTYNYVS | DVNQRPS | SSYGGSNNLV |
| | SEQ ID NO: 153 | SEQ ID NO: 163 | SEQ ID NO: 173 |
| BCMA32 | TGTSSDVGGYNYVS | DVSKRPS | SSYTSSSTVV |
| | SEQ ID NO: 147 | SEQ ID NO: 164 | SEQ ID NO: 174 |
| BCMA33 | TGTSSDVGGYNYVS | EVSKRPS | SSYAGSNNFVV |
| | SEQ ID NO: 147 | SEQ ID NO: 165 | SEQ ID NO: 175 |
| BCMA34 | TGNSNNVGNQGAA | RNNNRPS | SAWDSSLSARV |
| | SEQ ID NO: 154 | SEQ ID NO: 166 | SEQ ID NO: 176 |
| BCMA35 | SGSSSNIGSNTVN | SNNQRPS | AAWDDSLNGGV |
| | SEQ ID NO: 155 | SEQ ID NO: 159 | SEQ ID NO: 177 |
| Antibody | VH CDR1 | VH CDR2 | VH CDR3 |
| BCMA21 | SYAMS | AISGSGGSTYYADSVKG | ITAMVTHNFYGMDV |
| | SEQ ID NO: 178 | SEQ ID NO: 189 | SEQ ID NO: 201 |
| BCMA22 | SSNWWS | EIYHSGSTNYNPSLKS | VGSGYSYGYGDRVLDY |
| | SEQ ID NO: 179 | SEQ ID NO: 190 | SEQ ID NO: 202 |
| BCMA23 | SYWIG | IIYPGDSDTRYSPSFQG | RGGALDY |
| | SEQ ID NO: 180 | SEQ ID NO: 191 | SEQ ID NO: 203 |
| BCMA24 | SYAIS | GIIPIFGTANYAQKFQG | GSFYSSVNV |
| | SEQ ID NO: 181 | SEQ ID NO: 192 | SEQ ID NO: 204 |
| BCMA27 | SYGMH | VISYDGSNKYYADSVKG | EEFYGDSSYGMDV |
| | SEQ ID NO: 182 | SEQ ID NO: 193 | SEQ ID NO: 205 |
| BCMA28 | SNSAAWN | RTYYRSKWYNDYAVSVKS | DYYYGMDV |
| | SEQ ID NO: 183 | SEQ ID NO: 194 | SEQ ID NO: 206 |
| BCMA30 | SYWIG | IIYPGDSDTRYSPPFQG | LDASGSQRGGMDV |
| | SEQ ID NO: 180 | SEQ ID NO: 195 | SEQ ID NO: 207 |
| BCMA31 | SYWMS | NIKPDGSDKYYVDSVKG | GATTYGS |
| | SEQ ID NO: 184 | SEQ ID NO: 196 | SEQ ID NO: 208 |
| BCMA32 | RYAIS | GIIPFFGTSDYAQKFQG | RVATTGTGFYYAMDV |
| | SEQ ID NO: 185 | SEQ ID NO: 197 | SEQ ID NO: 209 |
| BCMA33 | DYAMH | GISWNSGSIGYADSVKG | VGASAALGAFDI |
| | SEQ ID NO: 186 | SEQ ID NO: 198 | SEQ ID NO: 210 |
| BCMA34 | NPFVH | IINLSGGDTLYAQKFQG | DQAGFGDSAY |
| | SEQ ID NO: 187 | SEQ ID NO: 199 | SEQ ID NO: 211 |
| BCMA35 | SYDIS | WINAYNGNANYAQKLQG | EYYYFWSNRAFYYGMDV |
| | SEQ ID NO: 188 | SEQ ID NO: 200 | SEQ ID NO: 212 |
| Antibody | VL CDR1 | VL CDR2 | VL CDR3 |
| C1 | TGTSSDIGAYNYVS | DVSHRPS | SSDTNNNTLV |
| | SEQ ID NO: 263 | SEQ ID NO: 293 | SEQ ID NO: 321 |
| C2 | SGSSSNIGNNYVS | DNNKRPS | VTWDTSLSAGV |
| | SEQ ID NO: 277 | SEQ ID NO: 305 | SEQ ID NO: 335 |
| C3 | RASQGIGTYLA | AASNLOS | QQYKGYPLT |
| | SEQ ID NO: 267 | SEQ ID NO: 297 | SEQ ID NO: 326 |
| C4 | SGSSSNIGSNYVN | RNNQRPS | AAWDDSLSGHGV |
| | SEQ ID NO: 278 | SEQ ID NO: 292 | SEQ ID NO: 336 |
| C5 | TRSSGSIAGSYVQ | QDNQRPS | QSYDSNNQV |
| | SEQ ID NO: 283 | SEQ ID NO: 310 | SEQ ID NO: 342 |
| C6 | SGADLGDQYVS | EDDKRPS | QAWDGNAAI |
| | SEQ ID NO: 284 | SEQ ID NO: 311 | SEQ ID NO: 343 |
| C7 | RASQSVSSSYLA | GASSRAT | QQYGSSPLT |
| | SEQ ID NO: 268 | SEQ ID NO: 298 | SEQ ID NO: 327 |
| C8 | TGTSTDIGGYDFVS | DVSSRPS | SSYAGSGTWM |
| | SEQ ID NO: 264 | SEQ ID NO: 294 | SEQ ID NO: 322 |
| C9 | SGSNSNIENNYVY | QNNQRPS | SAWDDSLSAWV |
| | SEQ ID NO: 285 | SEQ ID NO: 312 | SEQ ID NO: 344 |
| C10 | GGNNIGSKSVH | DDSDRPS | QVWDSSSDHWV |
| | SEQ ID NO: 286 | SEQ ID NO: 313 | SEQ ID NO: 345 |
| C11 | SGSSSNIGSNYVY | RNNQRPS | AAWDDSLSGYV |
| | SEQ ID NO: 262 | SEQ ID NO: 292 | SEQ ID NO: 320 |
| C12 | SGSSSNIGNNYVS | DDNKRPS | SAWDDSLSGYV |
| | SEQ ID NO: 277 | SEQ ID NO: 306 | SEQ ID NO: 337 |
| C13 | RSSQGLVFSDGVTYLN | KVSNRDS | MQGTHWPHT |
| | SEQ ID NO: 287 | SEQ ID NO: 314 | SEQ ID NO: 346 |
| C14 | QASQDITNYLN | DASNLET | QQYDDLPLT |
| | SEQ ID NO: 269 | SEQ ID NO: 299 | SEQ ID NO: 328 |
| C15 | RASQSISSWLA | KTSALED | LHYGSYT |
| | SEQ ID NO: 270 | SEQ ID NO: 300 | SEQ ID NO: 329 |
| C16 | SGSSSNIEGNTVS | SNSYRAS | SAWDDSLNGPV |
| | SEQ ID NO: 282 | SEQ ID NO: 309 | SEQ ID NO: 341 |
| C17 | TGTSSDVGTYDYVS | DVTYRPS | QQYNNWPPRYT |
| | SEQ ID NO: 265 | SEQ ID NO: 295 | SEQ ID NO: 323 |
| C18 | TGTSSDVGGYNYVS | DVSNRPS | SSYTSSSTLFV |
| | SEQ ID NO: 261 | SEQ ID NO: 290 | SEQ ID NO: 324 |
| C19 | RASQDIRHDLG | ATSTLQS | LQDGDYPLT |
| | SEQ ID NO: 271 | SEQ ID NO: 301 | SEQ ID NO: 330 |
| C20 | TGTSSDVGGYNYVS | EVSERPS | SSYAGSNILV |
| | SEQ ID NO: 261 | SEQ ID NO: 291 | SEQ ID NO: 319 |
| C21 | SGSSSNVGGNPVY | GHNQRPP | AAWDDSLSGWV |
| | SEQ ID NO: 279 | SEQ ID NO: 307 | SEQ ID NO: 338 |
| C22 | GANNIETKSVH | YDSDRPS | QVWDSTSDHVV |
| | SEQ ID NO: 288 | SEQ ID NO: 315 | SEQ ID NO: 347 |
| C23 | QASQDISNYLN | DASNLET | QQYGNLPLT |
| | SEQ ID NO: 272 | SEQ ID NO: 299 | SEQ ID NO: 331 |
| C24 | RASQSISSYLN | AASSLOS | LQYKTYPYT |
| | SEQ ID NO: 273 | SEQ ID NO: 302 | SEQ ID NO: 332 |
| C25 | TGTSSDVGGYNYVS | DVSNRPS | SSYTSSSTWV |
| | SEQ ID NO: 261 | SEQ ID NO: 290 | SEQ ID NO: 317 |
| C26 | SGSRSNIGSNTVS | LNNQRPS | ATWDDDLNGV |
| | SEQ ID NO: 280 | SEQ ID NO: 308 | SEQ ID NO: 339 |
| C27 | RASQRVSATYLA | GGSRRAT | QQYGSSPLT |
| | SEQ ID NO: 274 | SEQ ID NO: 303 | SEQ ID NO: 327 |
| C28 | TGTSSDVGGYNYVS | DVSNRPS | SSYTSSSNWV |
| | SEQ ID NO: 261 | SEQ ID NO: 290 | SEQ ID NO: 318 |
| C29 | SGSSSNIGSNYVY | RNNQRPS | AAWDDSLSGYV |
| | SEQ ID NO: 262 | SEQ ID NO: 292 | SEQ ID NO: 320 |
| C30 | QASQNINNYLN | DASNLEA | QQNDNLPLT |
| | SEQ ID NO: 275 | SEQ ID NO: 304 | SEQ ID NO: 333 |
| C31 | SGGNSNIGDNSVS | NNNQRPL | SSWDDSLEGAQ |
| | SEQ ID NO: 289 | SEQ ID NO: 316 | SEQ ID NO: 348 |
| C32 | SGSSSNIEGNTVS | SNSYRAS | SAWDDSLNGPV |
| | SEQ ID NO: 282 | SEQ ID NO: 309 | SEQ ID NO: 341 |
| C33 | RASQDISSWIA | AASSLOS | QQGSTFPLA |
| | SEQ ID NO: 276 | SEQ ID NO: 302 | SEQ ID NO: 334 |
| C34 | TGTRSDVGAYNYVS | DVSKRPS | SSYAGSGKYV |
| | SEQ ID NO: 266 | SEQ ID NO: 296 | SEQ ID NO: 325 |
| C35 | SGSSPNIGNNYVS | DNNKRPS | GTWDSRLNIWV |
| | SEQ ID NO: 281 | SEQ ID NO: 305 | SEQ ID NO: 340 |
| Antibody | VH CDR1 | VH CDR2 | VH CDR3 |
| C1 | SYGMH | VIWYDGSNKYYADSVKG | DRDGFFLPMYYFDY |
| | SEQ ID NO: 351 | SEQ ID NO: 372 | SEQ ID NO: 396 |
| C2 | NAWMS | RIKSKTDGGTTDYAAPVKG | DPHGYYYGMDV |
| | SEQ ID NO: 349 | SEQ ID NO: 382 | SEQ ID NO: 409 |
| C3 | SYAIS | GIIPIFGTANYAQKFQG | DSGWFGPRYYFDY |
| | SEQ ID NO: 355 | SEQ ID NO: 376 | SEQ ID NO: 400 |
| C4 | RYYMN | AIDPSDGDTTYGQKFQG | DWELTNSYGLDV |
| | SEQ ID NO: 360 | SEQ ID NO: 383 | SEQ ID NO: 410 |
| C5 | DYGMS | GINWNGGRTGYADSVKG | ARGSGSYFGYMDV |
| | SEQ ID NO: 359 | SEQ ID NO: 389 | SEQ ID NO: 417 |
| C6 | RYSMN | YISSSSSTIYYADSVKG | DGYGGNSDYYGMDV |
| | SEQ ID NO: 366 | SEQ ID NO: 388 | SEQ ID NO: 418 |
| C7 | SYGIS | WISAYNGNTNYAQKLQG | DPYDDYGGNSRDEEDAFDI |
| | SEQ ID NO: 356 | SEQ ID NO: 377 | SEQ ID NO: 401 |
| C8 | NAWMS | RIKSKTDGGTTDSAAPVKG | YYFYSSGDMTDY |
| | SEQ ID NO: 349 | SEQ ID NO: 370 | SEQ ID NO: 394 |
| C9 | DYAMN | FIRSKASGGTTDYAASVKG | ALRGYSYGLFDY |
| | SEQ ID NO: 367 | SEQ ID NO: 390 | SEQ ID NO: 419 |
| C10 | SYAIS | GIIPIFGTANYAQKFQG | EKWGYYGDYYYGMDV |
| | SEQ ID NO: 355 | SEQ ID NO: 376 | SEQ ID NO: 420 |
| C11 | DYAMS | FIRSKAYGGTTEYAASVKG | DIGYYGSGSYSPFDY |
| | SEQ ID NO: 350 | SEQ ID NO: 371 | SEQ ID NO: 395 |
| C12 | SYNIN | SISSSSSYRYYADSVKG | DKTGGYGTAFDY |
| | SEQ ID NO: 361 | SEQ ID NO: 384 | SEQ ID NO: 411 |
| C13 | SYSMN | FISHDASKKFYADSVKG | DRNWGFSDF |
| | SEQ ID NO: 365 | SEQ ID NO: 391 | SEQ ID NO: 421 |
| C14 | SSSHYWG | SIYYSGSTYYNPSLKS | RVWVWPTYGMDV |
| | SEQ ID NO: 357 | SEQ ID NO: 378 | SEQ ID NO: 402 |
| C15 | DYYIS | YISGSSTYTDFADSVKG | DRRGRFDP |
| | SEQ ID NO: 358 | SEQ ID NO: 379 | SEQ ID NO: 403 |
| C16 | SYSMN | YISSSSSTIYYADSVKG | DIYGDWDYYYYGMDV |
| | SEQ ID NO: 365 | SEQ ID NO: 388 | SEQ ID NO: 416 |
| C17 | SYYMH | LINPTNGRTTYAQKFQG | GSPPHIFDPDFDY |
| | SEQ ID NO: 352 | SEQ ID NO: 373 | SEQ ID NO: 397 |
| C18 | SGDYYWS | YIHSTGTTYYNPSLKS | GGVGTIFNY |
| | SEQ ID NO: 353 | SEQ ID NO: 374 | SEQ ID NO: 398 |
| C19 | SYGMH | VISYDGSNKYYADSVKG | DPSLIQYYYYGMDV |
| | SEQ ID NO: 351 | SEQ ID NO: 380 | SEQ ID NO: 404 |
| C20 | NAWMS | RIKSKTDGGTTDSAAPVKG | YYFYSSGDMTDY |
| | SEQ ID NO: 349 | SEQ ID NO: 370 | SEQ ID NO: 394 |
| C21 | DYAMH | GISWNSGSIGYADSVKG | GRYVDIVATINDAFDI |
| | SEQ ID NO: 362 | SEQ ID NO: 385 | SEQ ID NO: 412 |
| C22 | SYWIG | IINPSGGSTSYAQKFQG | VVTPYYPLTN |
| | SEQ ID NO: 368 | SEQ ID NO: 392 | SEQ ID NO: 422 |
| C23 | SYGIS | WISAYNGNTNYAQKLQG | DRIAAAEVYYYYGMDV |
| | SEQ ID NO: 356 | SEQ ID NO: 377 | SEQ ID NO: 405 |
| C24 | SYAIS | GIIPIFGTANYAQKFOG | DSPSGSTNYYYYYMDV |
| | SEQ ID NO: 355 | SEQ ID NO: 376 | SEQ ID NO: 406 |
| C25 | NAWMS | RIKSKTDGGTTDSAAPVKG | YYFYSSGDMTDY |
| | SEQ ID NO: 349 | SEQ ID NO: 370 | SEQ ID NO: 394 |
| C26 | NFEMN | YISATTSATSYADSVKG | VRDGYNFGYYYYYMDV |
| | SEQ ID NO: 363 | SEQ ID NO: 386 | SEQ ID NO: 413 |
| C27 | SYGIS | WISAYNGNTNYAQKLQG | DPYDDYGGNSRDEEDAFDI |
| | SEQ ID NO: 356 | SEQ ID NO: 377 | SEQ ID NO: 401 |
| C28 | NAWMS | RIKSKTDGGTTDSAAPVKG | YYFYSSGDMTDY |
| | SEQ ID NO: 349 | SEQ ID NO: 370 | SEQ ID NO: 394 |
| C29 | DYAMS | FIRSKAYGGTTEYAASVKG | DIGYYGSGSYSPFDY |
| | SEQ ID NO: 350 | SEQ ID NO: 371 | SEQ ID NO: 395 |
| C30 | DYGMS | GINWNGGSTGYADSVKG | DRAPDYGDYLQVGTTDY |
| | SEQ ID NO: 359 | SEQ ID NO: 381 | SEQ ID NO: 407 |
| C31 | DYYMS | YISSSGSTIYYADSVKG | GHQYYDFWSGYSNWFDP |
| | SEQ ID NO: 369 | SEQ ID NO: 393 | SEQ ID NO: 423 |
| C32 | SYAMN | DISSSSSYIYYADSVKG | DLYSGYDYGYYYGMDV |
| | SEQ ID NO: 364 | SEQ ID NO: 387 | SEQ ID NO: 415 |
| C33 | SYAIS | GIIPIFGTANYAQKFOG | GLDNWNDVGWFDP |
| | SEQ ID NO: 355 | SEQ ID NO: 376 | SEQ ID NO: 408 |
| C34 | DTWMS | RIKSKTDGGTTNYAAPVKG | GDYPLFAY |
| | SEQ ID NO: 354 | SEQ ID NO: 375 | SEQ ID NO: 399 |
| C35 | NAWMS | RIKSKTDGGTTDYAAPVKG | EAAGFYYYYYGMDV |
| | SEQ ID NO: 349 | SEQ ID NO: 382 | SEQ ID NO: 414 |

TABLE 4
Corresponding relationship between sequences and SEQ ID NO.

| SEQ | Amino acid | SEQ | Amino acid | SEQ | Amino acid |
|---|---|---|---|---|---|
| ID NO | sequence | ID NO | sequence | ID NO | sequence |
| 86 | M6 VL | 106 | M13 VH | 126 | M27 scFv |
| 87 | M7 VL | 107 | M15 VH | 127 | M28 scFv |
| 88 | M8 VL | 108 | M20 VH | 128 | M31 scFv |
| 89 | M10 VL | 109 | M22 VH | 129 | M32 scFv |
| 90 | M12 VL | 110 | M24 VH | 130 | M37 scFv |
| 91 | M13 VL | 111 | M27 VH | 131 | M6 CAR |
| 92 | M15 VL | 112 | M28 VH | 132 | M7 CAR |
| 93 | M20 VL | 113 | M31 VH | 133 | M8 CAR |
| 94 | M22 VL | 114 | M32 VH | 134 | M10 CAR |
| 95 | M24 VL | 115 | M37 VH | 135 | M12 CAR |
| 96 | M27 VL | 116 | M6 scFv | 136 | M13 CAR |
| 97 | M28 VL | 117 | M7 scFv | 137 | M15 CAR |
| 98 | M31 VL | 118 | M8 scFv | 138 | M20 CAR |
| 99 | M32 VL | 119 | M10 scFv | 139 | M22 CAR |
| 100 | M37 VL | 120 | M12 scFv | 140 | M24 CAR |
| 101 | M6 VH | 121 | M13 scFv | 141 | M27 CAR |
| 102 | M7 VH | 122 | M15 scFv | 142 | M28 CAR |
| 103 | M8 VH | 123 | M20 scFv | 143 | M31 CAR |
| 104 | M10 VH | 124 | M22 scFv | 144 | M32 CAR |
| 105 | M12 VH | 125 | M24 scFv | 145 | M37 CAR |
| 213 | BCMA21 | 229 | BCMA27 | 245 | BCMA32 |
| | VL | | VH | | scFv |
| 214 | BCMA22 | 230 | BCMA28 | 246 | BCMA33 |
| | VL | | VH | | scFv |
| 215 | BCMA23 | 231 | BCMA30 | 247 | BCMA34 |
| | VL | | VH | | scFv |
| 216 | BCMA24 | 232 | BCMA31 | 248 | BCMA35 |
| | VL | | VH | | scFv |
| 217 | BCMA27 | 233 | BCMA32 | 249 | BCMA21 |
| | VL | | VH | | CAR |
| 218 | BCMA28 | 234 | BCMA33 | 250 | BCMA22 |
| | VL | | VH | | CAR |
| 219 | BCMA30 | 235 | BCMA34 | 251 | BCMA23 |
| | VL | | VH | | CAR |
| 220 | BCMA31 | 236 | BCMA35 | 252 | BCMA24 |
| | VL | | VH | | CAR |
| 221 | BCMA32 | 237 | BCMA21 | 253 | BCMA27 |
| | VL | | scFv | | CAR |
| 222 | BCMA33 | 238 | BCMA22 | 254 | BCMA28 |
| | VL | | scFv | | CAR |
| 223 | BCMA34 | 239 | BCMA23 | 255 | BCMA30 |
| | VL | | scFv | | CAR |
| 224 | BCMA35 | 240 | BCMA24 | 256 | BCMA31 |
| | VL | | scFv | | CAR |
| 225 | BCMA21 | 241 | BCMA27 | 257 | BCMA32 |
| | VH | | scFv | | CAR |
| 226 | BCMA22 | 242 | BCMA28 | 258 | BCMA33 |
| | VH | | scFv | | CAR |
| 227 | BCMA23 | 243 | BCMA30 | 259 | BCMA34 |
| | VH | | scFv | | CAR |
| 228 | BCMA24 | 244 | BCMA31 | 260 | BCMA35 |
| | VH | | scFv | | CAR |
| 424 | C1 VL | 460 | C2 VH | 496 | C11 CAR |
| 425 | C2 VL | 461 | C3 VH | 497 | C1 scFv |
| 426 | C3 VL | 462 | C4 VH | 498 | C2 scFv |
| 427 | C4 VL | 463 | C5 VH | 499 | C3 scFv |
| 428 | C5 VL | 464 | C6 VH | 500 | C4 scFv |
| 429 | C6 VL | 465 | C7 VH | 501 | C5 scFv |
| 430 | C7 VL | 466 | C8 VH | 502 | C6 scFv |
| 431 | C8 VL | 467 | C9 VH | 503 | C7 scFv |
| 432 | C9 VL | 468 | C10 VH | 504 | C8 scFv |
| 433 | C10 VL | 469 | C11 VH | 505 | C9 scFv |
| 434 | C11 VL | 470 | C12 VH | 506 | C10 scFv |
| 435 | C12 VL | 471 | C13 VH | 507 | C11 scFv |
| 436 | C13 VL | 472 | C14 VH | 508 | C12 scFv |
| 437 | C14 VL | 473 | C15 VH | 509 | C13 scFv |
| 438 | C15 VL | 474 | C16 VH | 510 | C14 scFv |
| 439 | C16 VL | 475 | C17 VH | 511 | C15 scFv |
| 440 | C17 VL | 476 | C18 VH | 512 | C16 scFv |
| 441 | C18 VL | 477 | C19 VH | 513 | C17 scFv |
| 442 | C19 VL | 478 | C20 VH | 514 | C18 scFv |
| 443 | C20 VL | 479 | C21 VH | 515 | C19 scFv |
| 444 | C21 VL | 480 | C22 VH | 516 | C20 scFv |
| 445 | C22 VL | 481 | C23 VH | 517 | C21 scFv |
| 446 | C23 VL | 482 | C24 VH | 518 | C22 scFv |
| 447 | C24 VL | 483 | C25 VH | 519 | C23 scFv |
| 448 | C25 VL | 484 | C26 VH | 520 | C24 scFv |
| 449 | C26 VL | 485 | C27 VH | 521 | C25 scFv |
| 450 | C27 VL | 486 | C28 VH | 522 | C26 scFv |
| 451 | C28 VL | 487 | C29 VH | 523 | C27 scFv |
| 452 | C29 VL | 488 | C30 VH | 524 | C28 scFv |
| 453 | C30 VL | 489 | C31 VH | 525 | C29 scFv |
| 454 | C31 VL | 490 | C32 VH | 526 | C30 scFv |
| 455 | C32 VL | 491 | C33 VH | 527 | C31 scFv |
| 456 | C33 VL | 492 | C34 VH | 528 | C32 scFv |
| 457 | C34 VL | 493 | C35 VH | 529 | C33 scFv |
| 458 | C35 VL | 494 | C5 CAR | 530 | C34 scFv |
| 459 | C1 VH | 495 | C7 CAR | 531 | C35 scFv |

### Example 5: Assay of Cells Transduced with CAR or Co-Transduced with CAR and a LACOSTIM

CARs targeting different targets and CARTs are constructed as described in previous Examples 3-4, unless otherwise stated.

5.1 Mesothelin CART or Mesothelin CART Co-Transduced with CAR and a LACOSTIM
5.1.1: Cytokines Production of T Cells Co-Transduced with MSLN CAR and a LACOSTIM

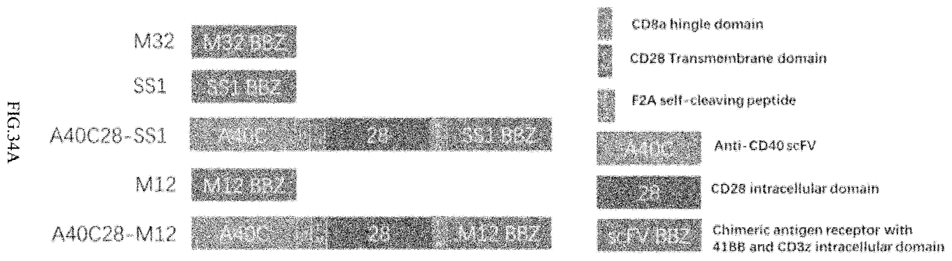

Lentiviral vectors co-expressing a LACOSTIM (A40C28, SEQ ID NO:600) and M12 or ss1, as well M12, M32 or ss1 CAR alone were constructed (FIG. 34A). T cells were transduced with these lentiviral vectors and the CAR expression was detected by flow cytometry (FIG. 34B). The function of the T cells transduced with the lentiviral vectors was tested by co-culturing with MSLN negative tumors PC3 expressing lower levels or higher levels MSLN (FIG. 34C Upper panel) or MSLN positive tumor lines OVCAR3 and H266 (FIG. 34C Lower panel) for the specificity and activity test. The results shown that background cytokine secretions of A40C28-M12 was slightly higher than that of M12 alone, but was lower than that of ss1 alone, when the T cells were tested on MSLN lower expressing tumors PC3 or PC3+Meso 0.5 ug. Indicating that T cells co-expressing LACO-STIM and M12 CAR is safer and more specifically recognize MSLN high expressing tumors than previously clinically used ss1 CAR.
5.1.2: Specific Activation of Anti-Mesothelin CART Cells and LACO-Expressing Mesothelin CART Cells by Mesothelin-Expression Cancer Cells

CD107a is an early phase-activating marker for T cells. Activation of mesothelin CARTs by mesothelin-expressing tumor cells was measured by CD107a staining with the following procedures: 20 μl PE-CD107a mAb was added to each well of a 96-well plate; tumor cells were diluted to 2×10e6/ml and seeded on 96-well round plates (100 μl/well); CAR-T cells were diluted to 1×10e6/ml and seeded in 96-well round plates (100 μl/well); the plates were centrifuged at 500 rpm×5 min to attach cells and cultured at 37° C. for 1 hour; Golgi stop was diluted by 1500× with medium and added to each well (20 μl/well); cells were cultured at 37° C. for another 2.5 hours, stained with anti-CD3-APC and anti-CD8-FITC antibodies at 37° C. for 30 min, washed and analyze by flow cytometry.

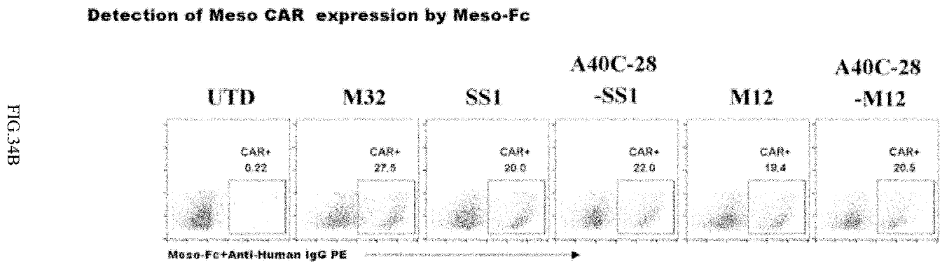

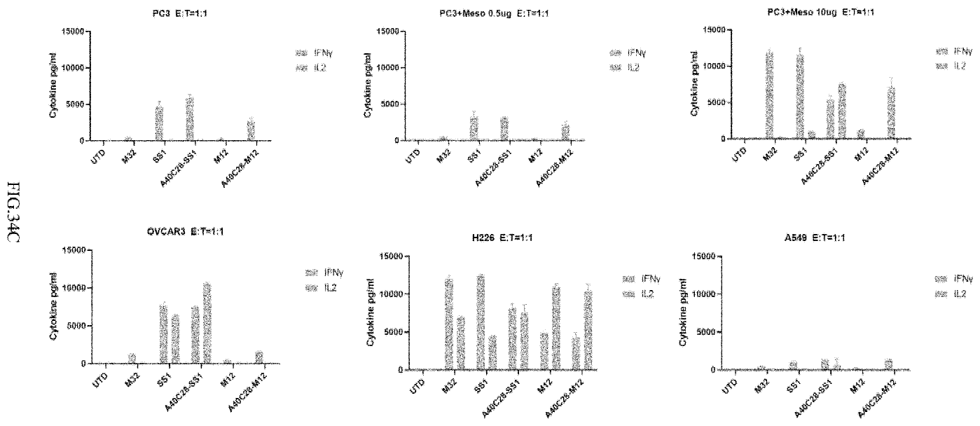

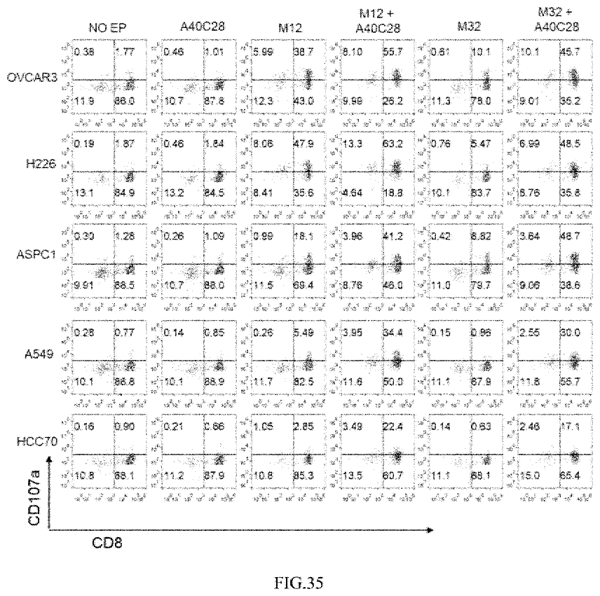

CART cells co-expressing a LACOSTIM (e.g., A40C28; SEQ ID NO:600) were also prepared and their activation by tumor cells were confirmed by CD107a staining. Various mRNA-based CART cells were prepared, including mock T cells (NO EP), T cells with A40C28, anti-mesothelin M12 CART cells (M12 CART), M12 CART cells co-expressing A40C28 (M12+A40C28 CART), M32 CART cells, and M32+A40C28 CART cells. These cells were cocultured with various cancer cell lines (OVCAR3, H226, ASPC1, A549 and HCC70) and CD107a expression was measured by flow cytometry. As shown in FIG. 35, anti-mesothelin M12 and M32 CAR-T cells were specifically activated by OVCAR3, H226, and ASPC1 (tumor cells with high mesothelin-expression level), but not A549 and HCC70 (tumor cell lines with low or no mesothelin expression).

### 5.1.3: Tumor Killing of LACO-Expressing Mesothelin CART Cells

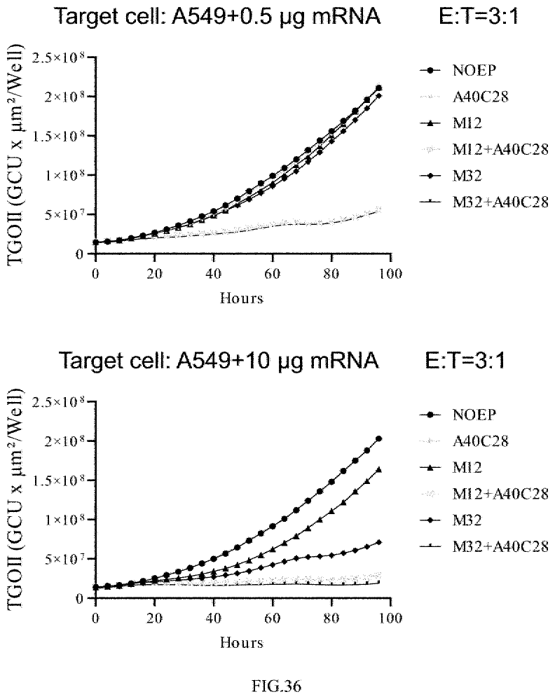

The tumor killing effects of the provided mesothelin CARTs cells were measured in the tumor killing assay. Various mRNA-based anti-mesothelin CAR-T cells, including mock T cells (NO EP), T cells with A40C28, anti-mesothelin M12 CART cells, M12 CART cells co-expressing A40C28, M32 CART cells, and M32 CART cells co-expressing A40C28 were co-cultured with A549-GFP tumor cells that were electroporated with 0, 0.5 μg and 10 μg mesothelin mRNA at E/T ratio=3:1. As shown in FIG. 36, anti-mesothelin scFv-M12 and -M32 CART cells had low killing effect toward the A549 tumor cells with low mesothelin expression (0.5 μg group) and strong killing effect toward A549 tumor cells with high mesothelin expression (10 μg group), while A40C28 greatly improved the killing efficiency of CART cells to mesothelin-expressing tumor cells.

Lentivirus-based CART cells were generated using the following procedures: T cells were isolated from PBMC and activated by anti-CD3/CD28 beads (T cell:beads=1:3). At day 1, the activated T cells were transduced with lentivirus at a multiplicity of infection (MOI) of 3. At day 7, the transduction efficiency of T cell was evaluated by FACS staining. Generally, the transduction efficiency was between 10% to 70%. The CART cells were cultured up to day 14, which were used for functional study immediately or frozen and stored using liquid nitrogen.

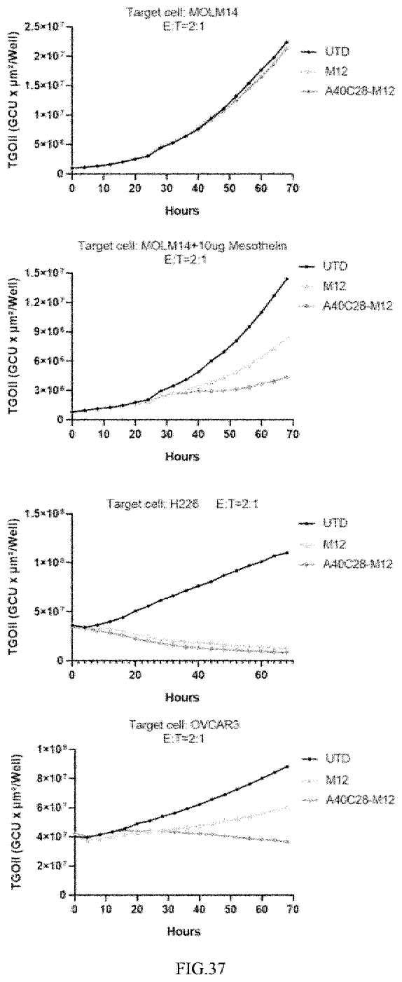

The tumor killing effects of the lentivirus-based anti-mesothelin CART cells were measured. CART cells including mock T cells (UTD), M12 CART cells, M12+A40C28 CART cells were co-cultured with different cancer cells, including H226, OVCAR3 and MOLM14 that electroporated with 0 or μg ug mesothelin mRNA at E/T ratio=2:1. As shown in FIG. 37, both M12 CART cells and M12+A40C28 CART cells showed strong killing effects toward mesothelin-expressing cancer cells, and the co-expression of A40C28 greatly improved the killing efficiency.

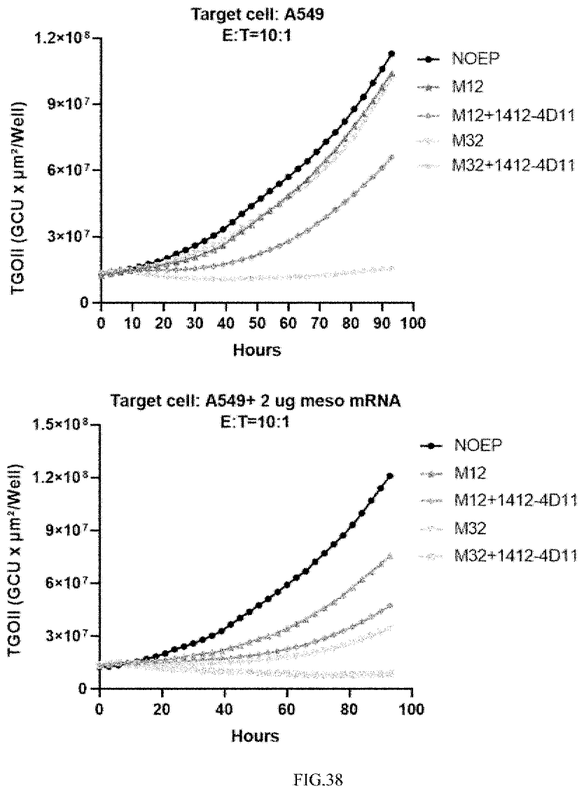

A second molecule LACOSTIM (1412-4D11, SEQ ID NO: 813) was also prepared and used in the studies. Various mRNA-based anti-mesothelin CART cells were prepared, including mock T cells (NO EP), M12 CART cells, M12+1412-4D11 CART cells, M32 CART cells, and M32+1412-4D11 CART cells. These CART cells were co-cultured with A549-GFP tumor cells that were electroporated with 0 or 2 μg mesothelin mRNA at E/T ratio=10:1. As shown in FIG. 38, both M12 and M32 CART cells demonstrated effective killing toward the mesothelin-expressing A549 tumor cells (2 μg group), and the co-expression of 1412-4D11 greatly improved the killing efficiency of the CART cells.

5.1.4: Lytic Activity of T Cells Co-Transduced with MSLN CAR and a LACOSTIM

The function of the T cells transduced with the lentiviral vectors was also tested for their killing ability against tumor cell lines expressing MSLN at different levels (0.2 ug MSLN RNA transferred PC3 or MOLM14, 10 ug MSLN RNA transferred PC3 or MOLM14, MSLN positive tumor OVCAR3 and H226). As shown in FIG. 39, For MSLN negative tumors PC3 or MOLM14, M12 and M12-A40C28 showed less background killings than M32 CAR, ss1 CAR and ss1 CAR+A40C28. For lower MSLN expressing tumor PC3+0.5 ug MSLN and MOLM14+0.5 ug MSLN, M12 and M12-A40C28 also shown much less background killings than M32 CAR, ss1 CAR and ss1 CAR+A40C28. While for MSLN high expressing tumors, M12+A40C28 shown effective tumor killing, albeit less effective than ss1 or ss1+A40C28. Therefore, M12+A40C28 CAR showed better safety profile than ss1 CAR.

### 5.1.5: Effective Killing of a Variety of Tumor Cells by Mesothelin CARTs

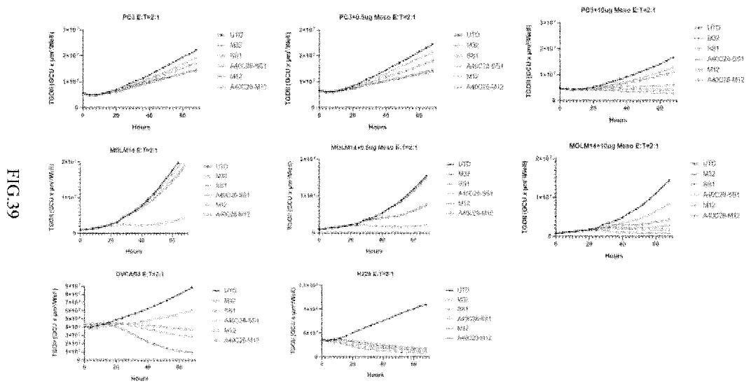

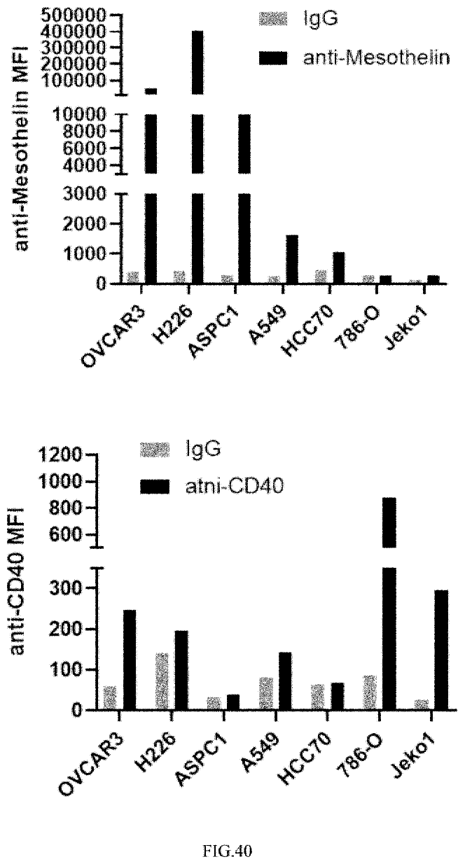

The tumor killing effects of the mesothelin CART cells were further confirmed in additional tumor cells. The mesothelin expression and CD40 expression in tumor cells (OVCAR3, H226, ASPC1, A549, HCC70, 786-0 and Jeko1) were measured by FACS staining using isotype control, anti-mesothelin mAb and anti-CD40 mAb. As shown in FIG. 40, OVCAR3, H226 and ASPC1 expressed mesothelin at high levels; A549, HCC70, 786-0 and Jeko1 expressed mesothelin at low levels. Also, OVCAR3, 786-0 and Jeko1 expressed CD40 at high levels, while H226, A549, HCC70 and ASPC1 expressed CD40 at low or no expression level.

Various mRNA-based anti-mesothelin CART cells, including mock T cells (NO EP), T cells with A40C28 alone, T cells with 1412-4D11 alone, M12 CART cells, M12+A40C28 CART cells, M12+1412-4D11 CART cells, M32 CART cells, M32+A40C28 CART cells, and M32+1412-4D11 CAR-T cells, were separately cocultured with OVCAR3, H226, ASPC1, A549, HCC70, 786-0 or Jeko1 tumor cell lines. As shown in CD107a staining results provided in FIG. 41, anti-mesothelin M12 and M32 CART cells were specifically activated by tumor cells with high mesothelin-expression level, but not tumor cell lines with low or no mesothelin expression. Both A40C28 and 1412-4D11 significantly improved the killing efficiency of CART cells against mesothelin and/or CD40 expressing tumor cells.

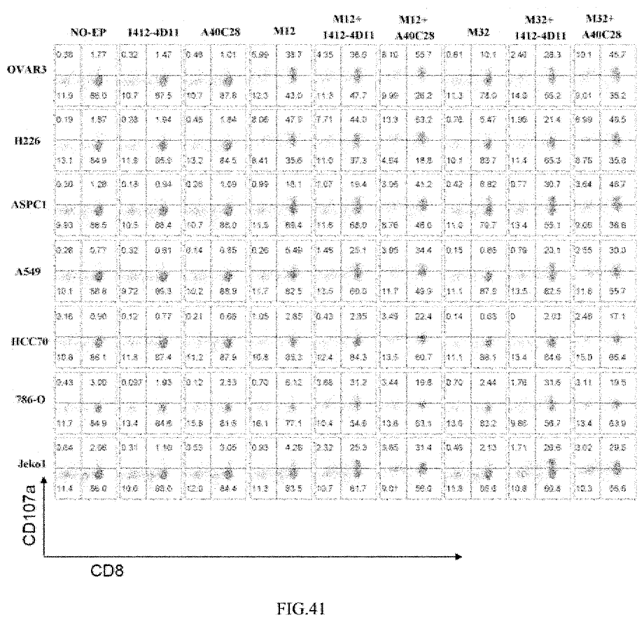

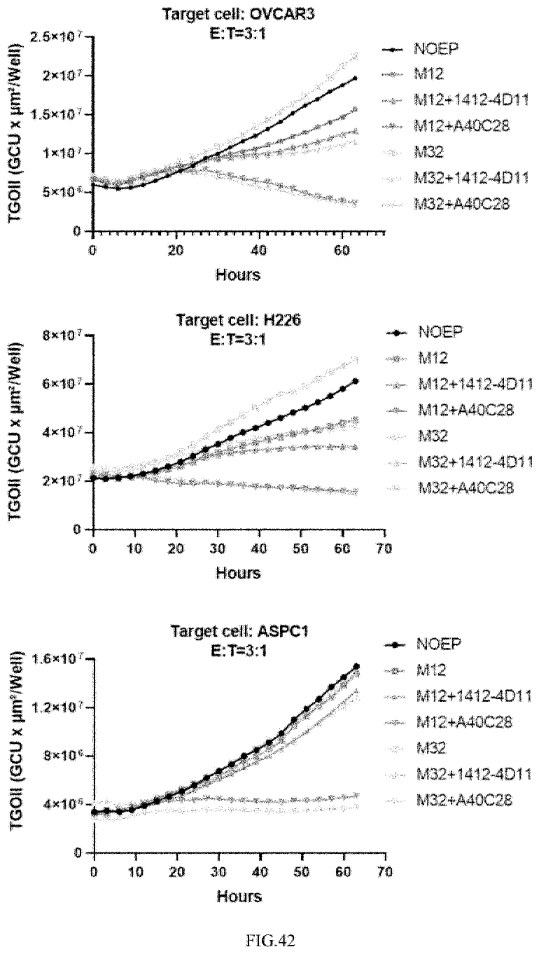

As further shown in the killing curves on FIG. 42, various mRNA-based anti-mesothelin CART cells, including mock T cells (NO EP), M12 CART cells, M12+A40C28 CART cells, M12+1412-4D11 CAR-T cells, M32 CART cells, M32+A40C28 CART cells, and M32+1412-4D11 CART cells were separately cocultured with OVCAR3-GFP, H226-GFP or ASPC1 tumor cells at E/T ratio=3:1. As shown, anti-mesothelin scFv-M12 and -M32 CART cells had moderate killing effect toward mesothelin overexpressing A549 tumor cells, and co-expression of either 1412-4D11 or A40C28 greatly improved the killing efficiency of these mesothelin CART cells against mesothelin and/or CD40 expression tumor cells.

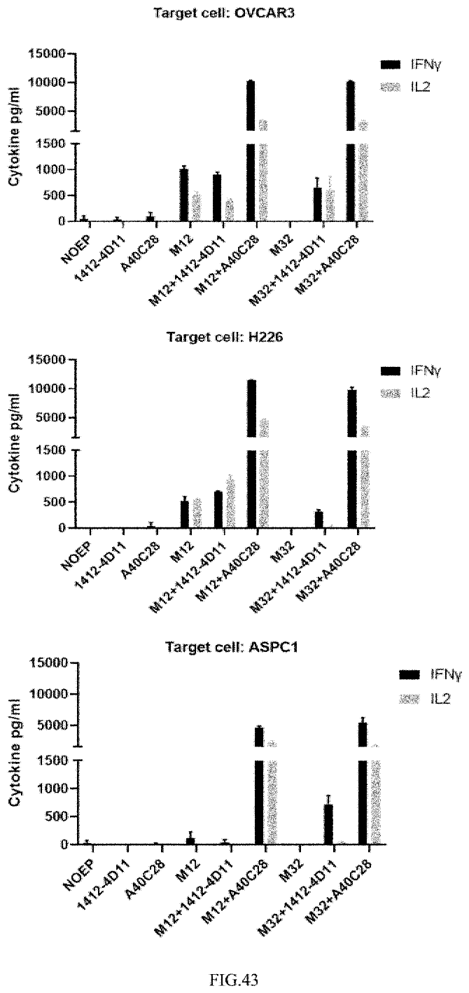

IFN-γ and IL2 release was detected by ELISA in the CART killing assay. Various mRNA-based anti-mesothelin CART cells, including mock T cells (NO EP), M12 CART cells, M12+A40C28 CART cells, M12+1412-4D11 CART cells, M32 CART cells, M32+A40C28 CART cells, and M32+1412-4D11 CAR-T cells, were separately cocultured with OVCAR3-GFP, H226-GFP or ASPC1 tumor cells at E/T ratio=1:1. As shown in FIG. 43, co-expression with LACO, either A40C28 or 1412-4D11, significantly enhanced the release of IFN-γ and IL2 by the CART cells when stimulated by all tested cancer cells.

5.1.6: Specificity Test of T Cells Co-Transduced with MSLN CAR and a LACOSTIM

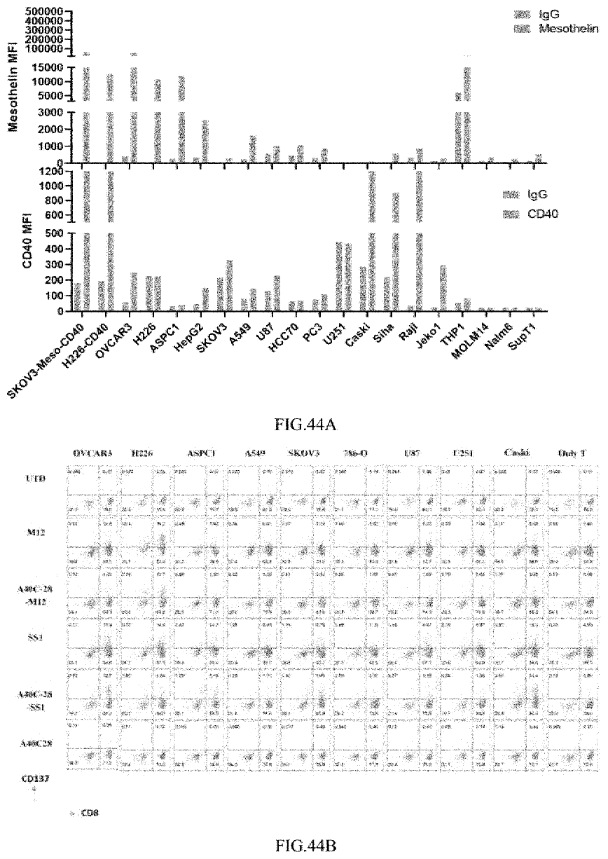

The expression of MLSN or CD40 of a panel of tumors was examined by flow cytometry (FIG. 44A). T cells transduced with the lentiviral vectors were stimulated with tumor cell lines expressing MSLN and CD40 at different levels and CD137 upregulation was examined by flow cytometry. It was found that M12 or A40C28-M12 CART cells were only reactive to MSLN high expression tumor OVCAR3 and H266, while the background activity of ss1 or ss1-A40C28 CART cells against some of MSLN low expressing tumors was higher than the M12 based CART cells (FIG. 44B).

### 5.1.7: Anti-Tumor Efficacy of A40C28-M12 LVV CART in H226 Xenograft NOG Tumor Model

To verify the anti-tumor potency and efficacy of A40C28-M12 lentiviral vector (LVV) CART in vivo, the NSG mice were subcutaneously injected (s.i.) with the 5E6 H226 tumor cells transduced with click beetle green (H226-CBG). 11 days later, mice were infused with 1E6 or 5E6 CAR positive T cells (i.v.) as indicated (FIG. 45A-45B). Tumor volumes (FIG. 45C) and average radiance of Bioluminescence (FIG. 45D) were measured at different time points. The results showed that both M12 CAR and A40C28-M12 CAR groups at 5M do control tumor growth efficiently, with A40C28-M12 CAR slightly more efficient than M12 CAR alone. At 1M dose, A40C28-M12 CART slightly and transiently controlled the tumor growth, whereas M12 failed to control tumor growth as non-transduced T cells (UTD) and A40C28 alone groups.

### 5.1.8: Anti-Tumor Efficacy of A40C28-M12 LVV CART in H226 Xenograft NOG Tumor Model

Tumor-infiltrating lymphocytes (TILs), located in the tumor microenvironment, are the fundamental elements of the specific immunological response against tumor cells and have prognostic importance in many types of cancer. To discovery the influence of A40C28 LACOSTIM on the TILs, TILs were isolated from the mice that were treated for 2 weeks (FIG. 46A). Distribution of CD4+ T cell, CD8+ T cell, Granzyme B+ T cell and MSLN+ target cells in tumor tissue were detected by multiplexed immunohistochemical (mIHC) (FIG. 46B). The results indicated that A40C28 LACO promote TIL (CD4+ and CD8+) infiltration and/or proliferation in treated H226-xenograft model (FIG. 46C).

5.2 BCMA CART or BCMA CART Co-Transduced with CAR and a LACOSTIM

### 5.2.1: Expression of BCMA by Tumor Cells

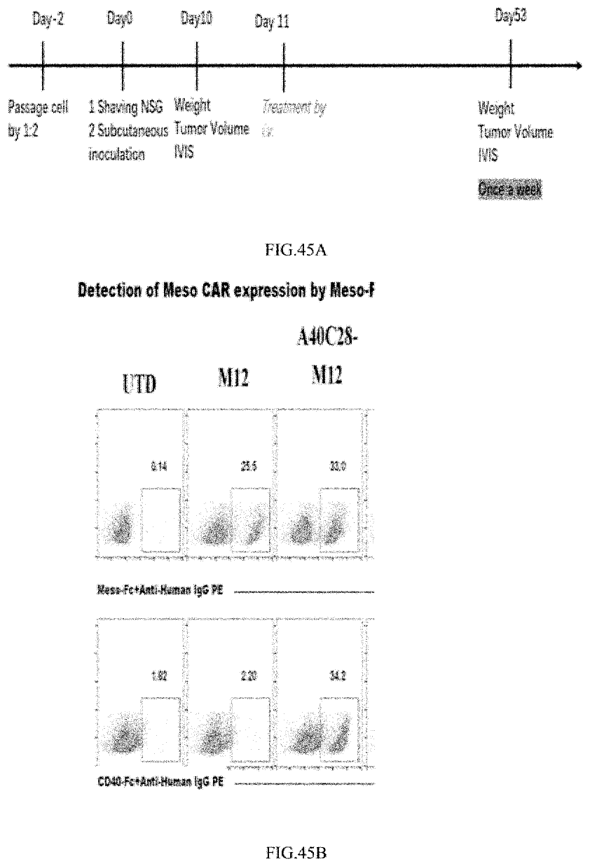

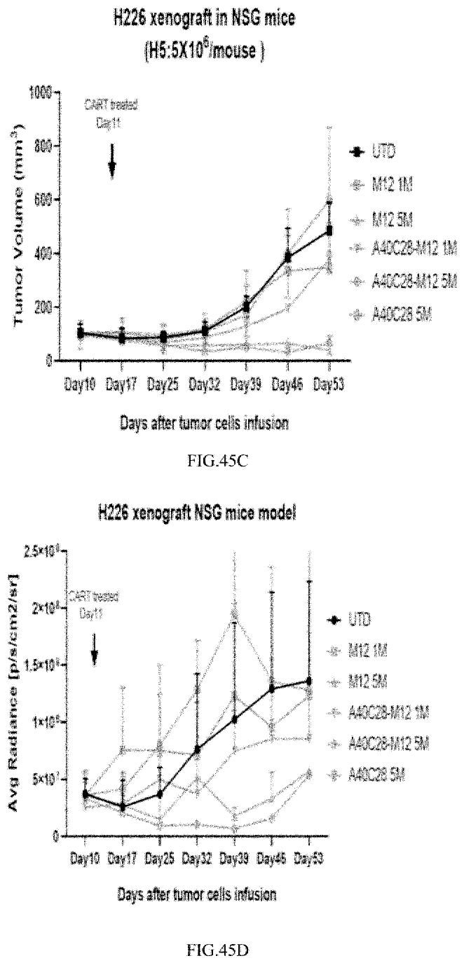

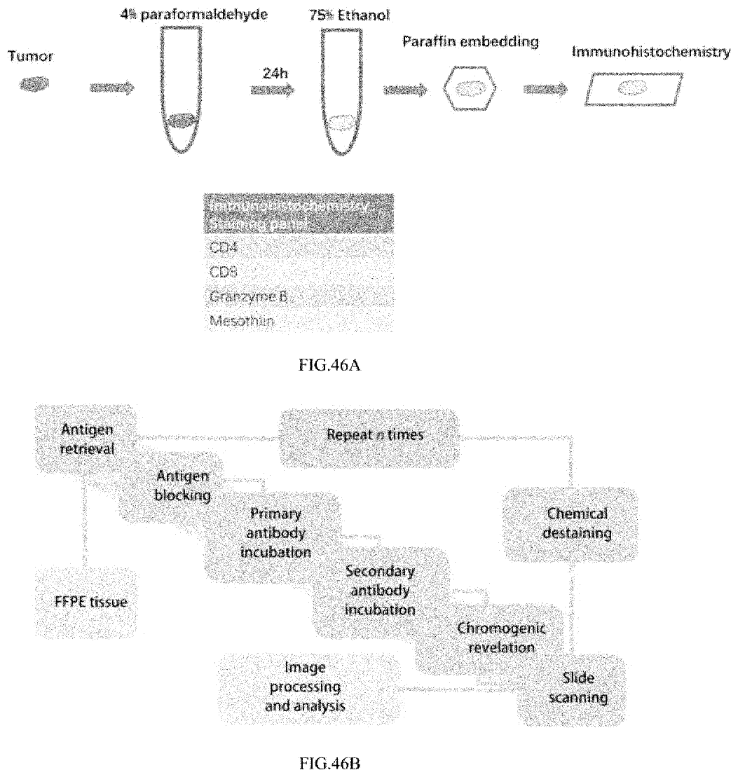

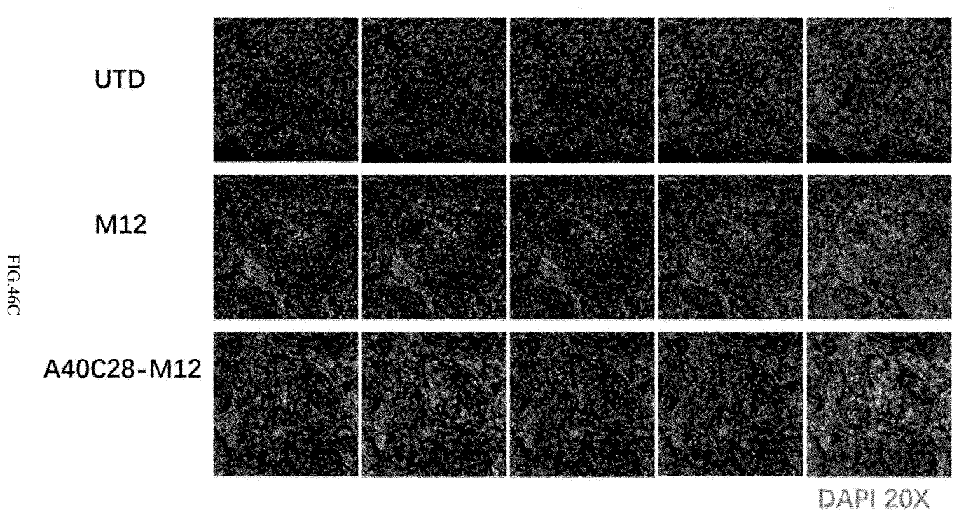

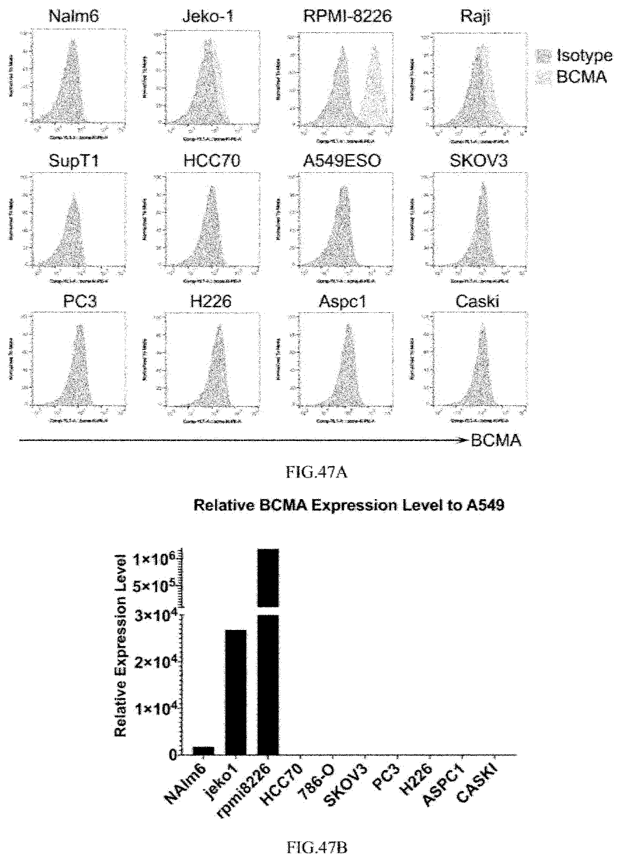

As shown in FIGS. 47A and 47B, different tumor cell lines were examined for the expression of BCMA by FACS staining (FIG. 47A) and RT-PCR (FIG. 47B). BCMA expression was detected in Jeko-1 (low level), Raji (intermediate level) and RPMI-8226 cells (high level) by FACS staining. Although BCMA expression was not detected in Nalm6 by FACS, RT-PCR analysis showed it was expressed, albeit at a very low level.

### 5.2.2: BCMA CART Showed Cytotoxicity Against Tumor Cells

The CART cells were cocultured with Jeko-1 cells and RPMI-8226 tumor cells. The production of INF-γ and IL-2 were examined. As shown in FIGS. 48A (INF-γ) and 48B (IL-2), of the 12 CARTs that we generated, BCMA23 (#5; SEQ ID NO:251), BCMA24 (#6; SEQ ID NO:252), BCMA27 (#7; SEQ ID NO:253), BCMA31 (#10; SEQ ID NO:256), and BCMA33 (#12; SEQ ID NO:258) produced more cytokines than the others, including NBC10 and B38M CAR T cells.

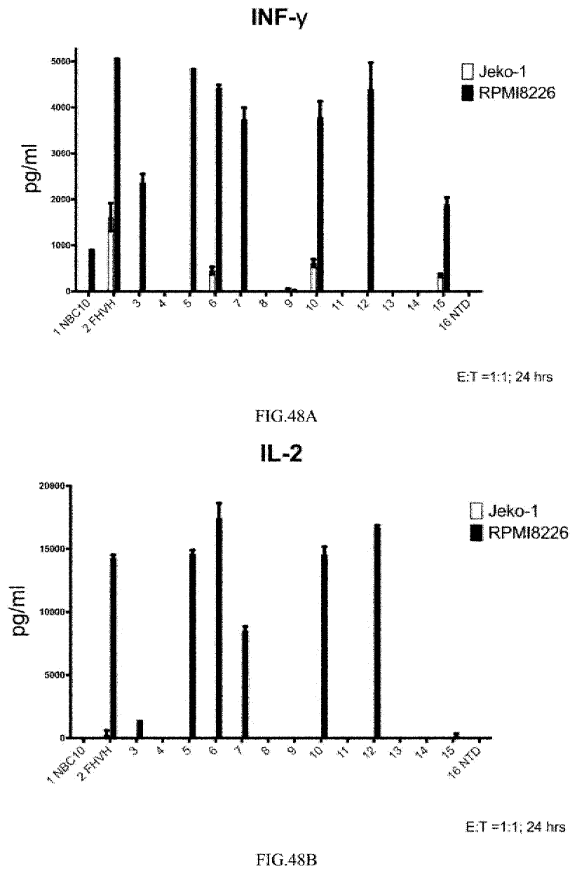

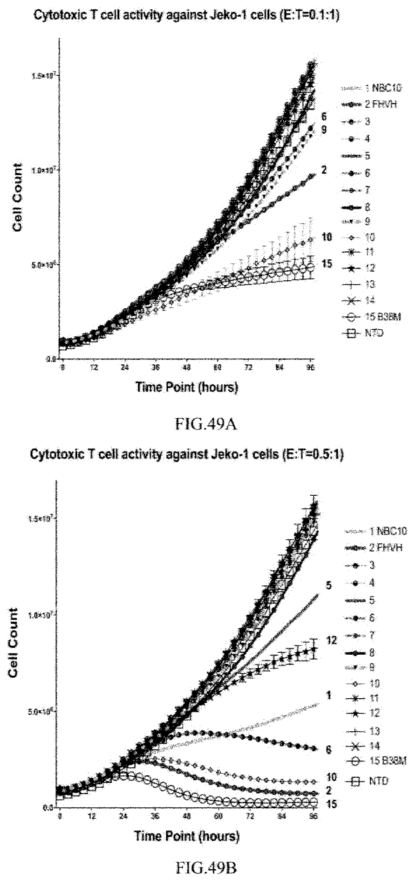

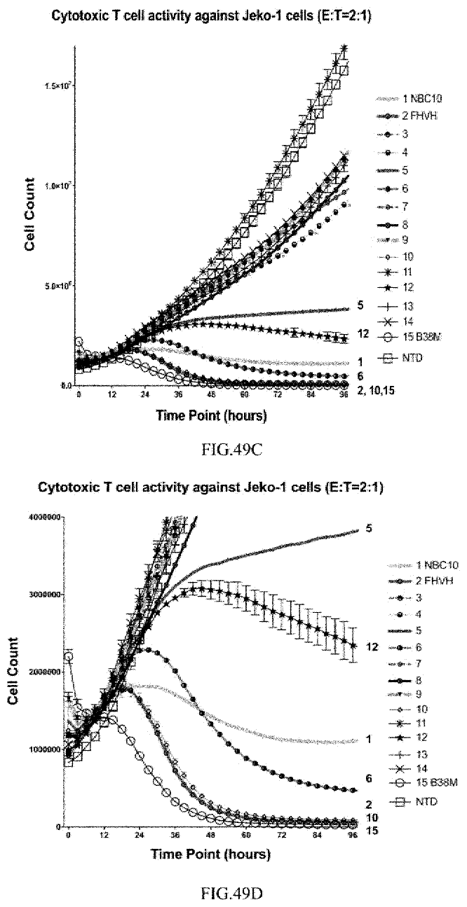

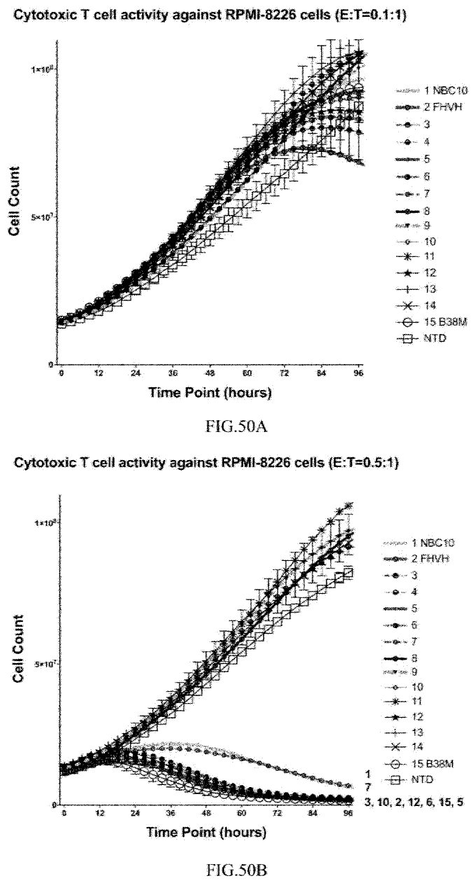

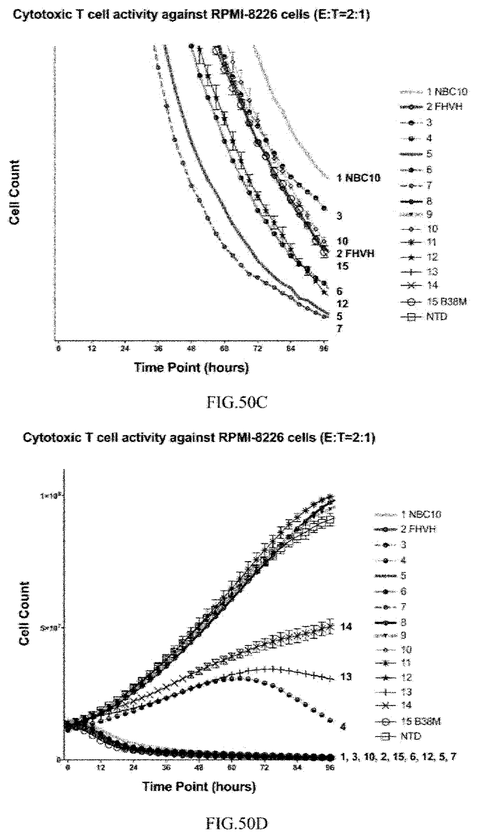

We also examined the cytolytic activities of the CART cells against Jeko-1 (FIGS. 49A-49D) and RPMI-8226 cells (FIGS. 50A-50E), respectively. BCMA23 (#5), BCMA24 (#6), BCMA31 (#10), and BCMA33 (#12) CART cells showed different levels of cytotoxicity against Jeko-1 cells, with BCMA31 (#10) being the highest, which efficiently eliminated Jeko-1 (FIGS. 49C-49D). Additionally, BCMA21 (#3; SEQ ID NO:249), BCMA22 (#4; SEQ ID NO:250), BCMA23 (#5; SEQ ID NO:251), BCMA24 (#6; SEQ ID NO:252), BCMA27 (#7; SEQ ID NO:253), BCMA31 (#10; SEQ ID NO:256), BCMA33 (#12; SEQ ID NO:258), BCMA34 (#13; SEQ ID NO:259), and BCMA35 (#14; SEQ ID NO:260) showed different levels of cytotoxicity against RPMI-8226 cells, of which BCMA21 (#3), BCMA23 (#5), BCMA24 (#6), BCMA27 (#7), BCMA31 (#10), and BCMA33 (#12) efficiently eliminated RPMI-8226 cells.

### 5.2.2: Armored BCMA31 CAR T Cells Showed Superior Anti-Tumor Activities

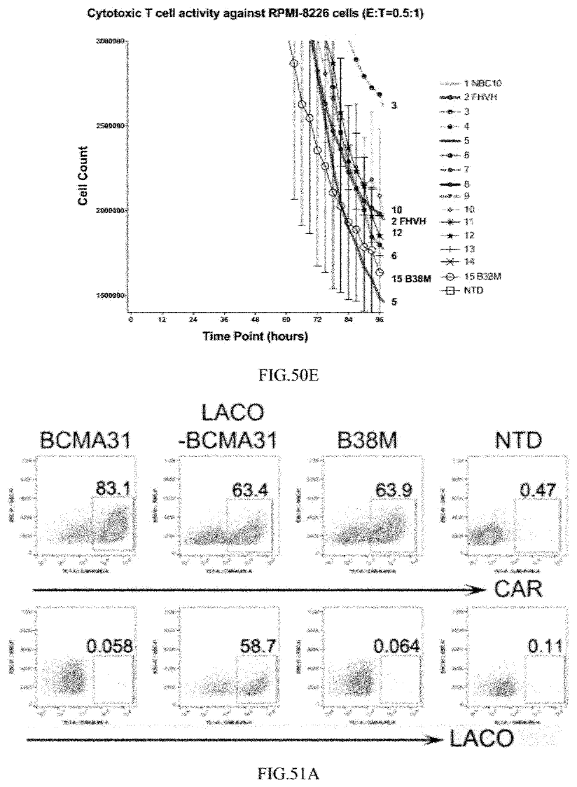

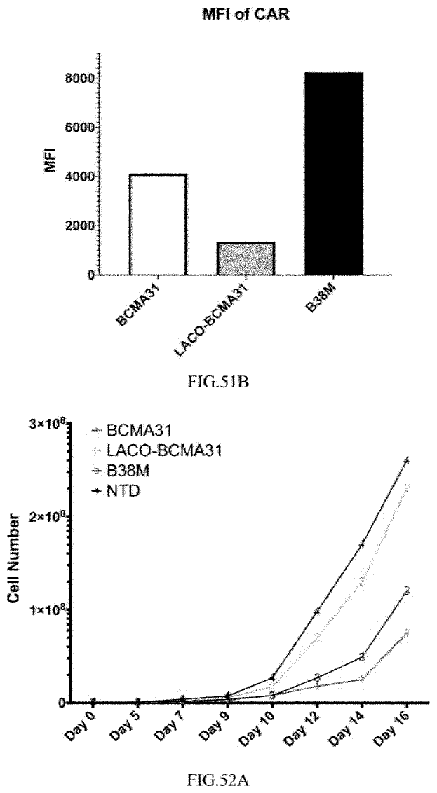

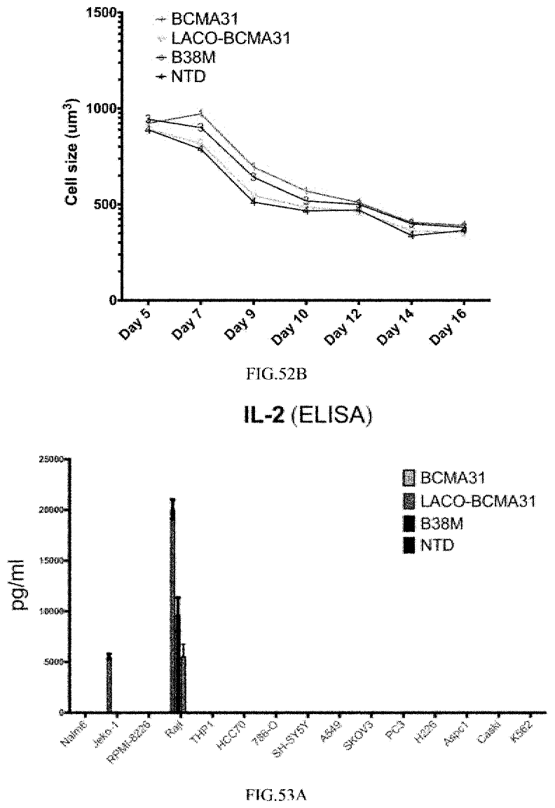

LACO (e.g., A40C.CD28 as used in this study) is a switch receptor that can engage CD40 antigen and then activate CD28 signaling in T cells. In this study, LACO-BCMA31 CART cells, BCMA31 CAR T cells and B38M CAR T were generated as follows. First, lentiviruses were generated and transduced to T cells to express BCMA31.BBz (SEQ ID NO:256), LACO-BCMA31.BBz (SEQ ID NO:601), and B38M.BBz. As shown in FIGS. 51A-51B, the expression level of B38M.BBz was the highest. The expression level of BCMA31.BBz in the LACO-BCMA31.BBz construct was lower than that of BCMA31.BBz-only construct. Also, the expansion of LACO-BCMA31 CART was much faster than that of BCMA31 CART and B38M CART (FIG. 52A), and the size of BCMA31 and B38M CART cells were larger than LACO-BCMA31 and NTD T cells (FIG. 52B).

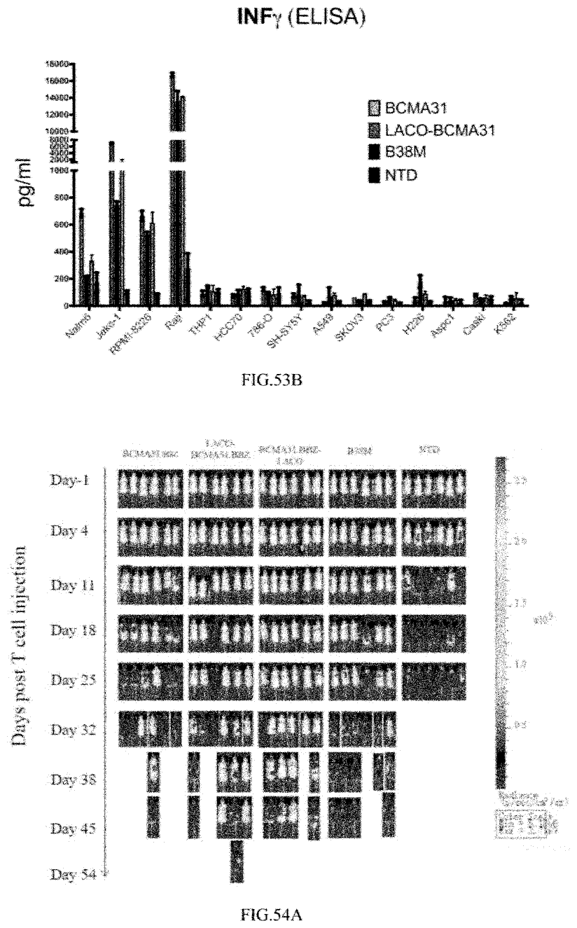

We cocultured these T cells with a panel of tumor cells and examined the production of INF-γ and IL-2 by the T cells. LACO-BCMA31 T cells produced significantly more IL-2 than other T cell types when cocultured with Jeko-1 and Raji (FIG. 53A). LACO-BCMA31 T cells also produced significantly more INF-γ than other T cell types when cocultured with Nalm6, Jeko-1 and Raji (FIG. 53B).

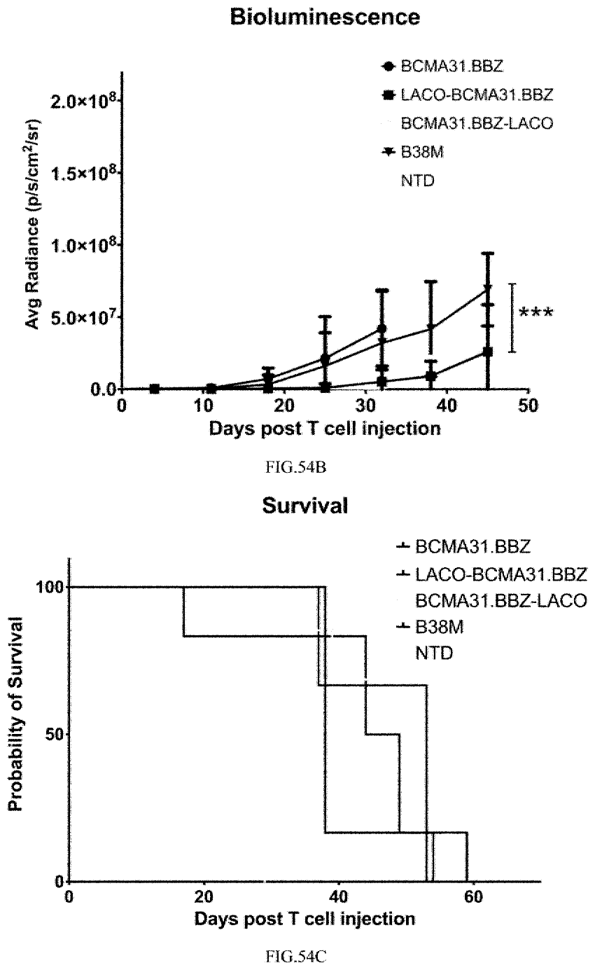

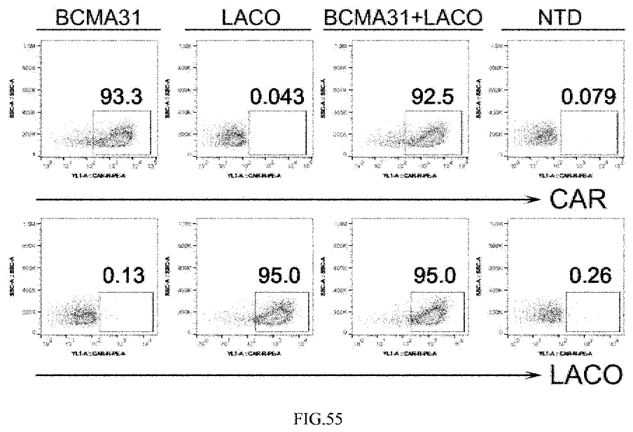

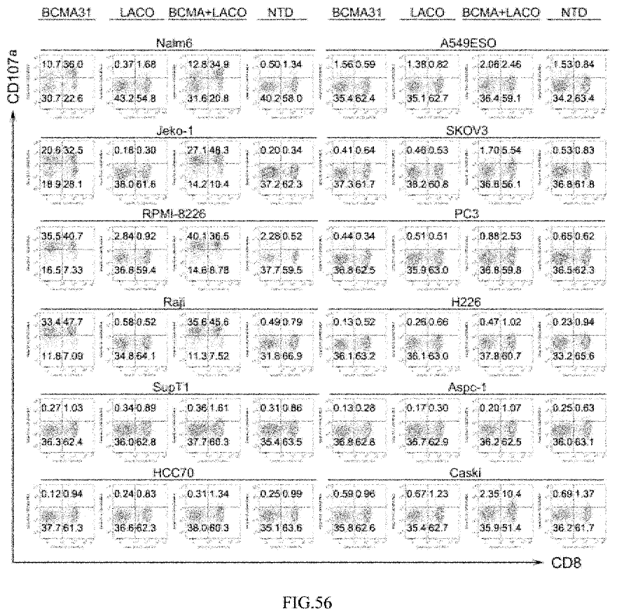

We evaluated the function of these CAR T cells in vivo. Jeko-1 tumor cells were established in NSG mice by intravenous injection. Nine days later, T cells were injected intravenously. Bioluminescence imaging showed that BCMA31 (SEQ ID NO:256), LACO-BCMA31 (SEQ ID NO:601), BCMA31-LACO (SEQ ID NO:602), and B38M T cells significantly reduced tumor growth. LACO-BCMA31 and BCMA31-LACO T cells had the greatest anti-tumor effect (FIGS. 55A-54C).

Next, we electroporated BCMA31.BBz, LACO, or both BCMA31.BBz and LACO mRNA into T cells for transient expression. The expression of CAR and LACO in the T cells was shown in FIG. 55 and Table 5 below.

TABLE 5
T cells electroporated with mRNA products

| | | BCMA | CAR | LACO | LACO |
|---|---|---|---|---|---|
| | Constructs | CAR % | MFI | % | MFI |
| 1 | BCMA31.BBz (10 μg) | 91.3% | 2238 | | |
| 2 | A40C.CD28 (10 μg) | | | 95.8% | 1244 |
| 3 | A40C.CD28-BCMA31.BBz | 90.5% | 2766 | 95.8% | 1234 |
| | (10 μg + 10 μg) | | | | |
| 4 | NTD | | | | |

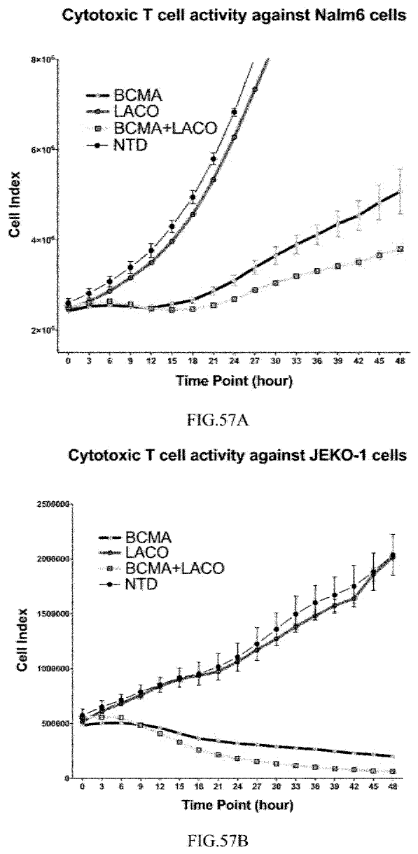

We cocultured T cells with different tumor cells for four hours and then examined the activation of the T cells by tumor cells. CD107a were strongly activated in the BCMA31 T cells and BCMA31+LACO T cells when they were cocultured with BCMA+ tumor cells, including Nalm6, Jeko-1, RPMI-8226, and Raji (FIG. 56).

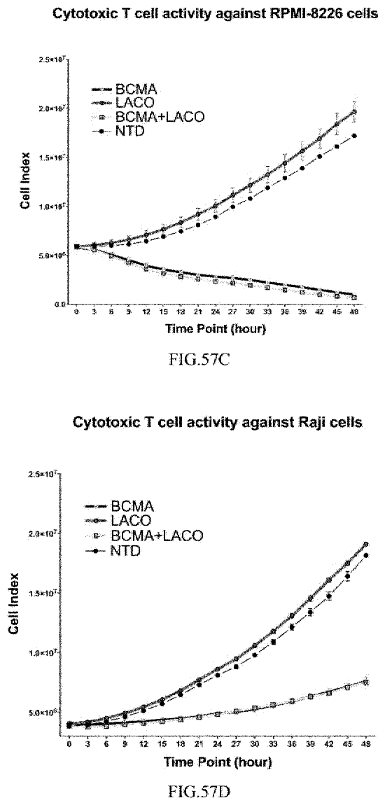

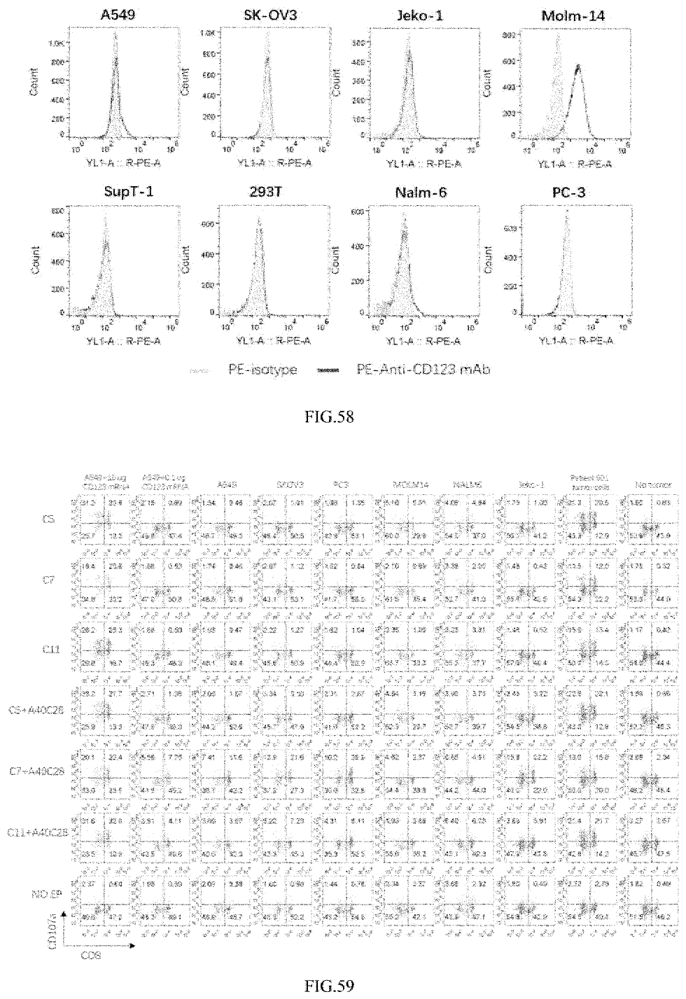

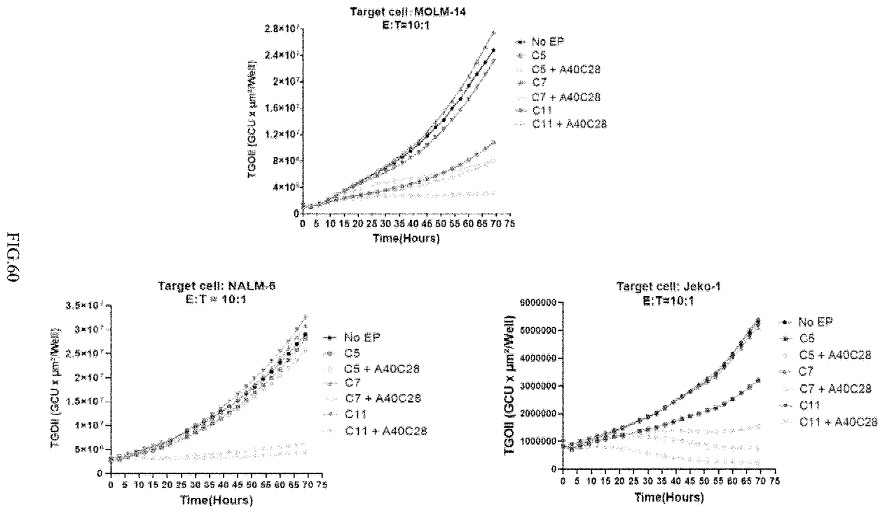

The cytotoxic T cell activities against the tumor cells were further confirmed by Incucyte Live-Cell Analysis System. BCMA31 T cells and BCMA+LACO T cells effectively controlled the growth of BCMA+ tumor cells compared with NTD and LACO alone T cells (FIGS. 57A-57D).

5.3 CD123 CART or CD123 CART Co-Transduced with CAR and a LACOSTIM

### 5.3.1: CD107a Staining of CART Cells.

The CD123 expression by various tumor cell lines were measured by FACS staining. FIG. 58 shows the FACS staining of A549, SK-OV3, Jeko-1, Molm-14, SupT-1, 293T, Nalm-6 and PC-3 cells with PE-isotype control and PE-anti-CD123 mAb. As shown, most tested tumor cell lines did not express CD123, with only Molm-14 expressing CD123 at a relatively high level.

CD107a is an early phase-activating marker for T cells. Activation of CD123 CARTs by CD123-expressing tumor cells was measured by CD107a staining with the following procedures: 20 μl PE-CD107a mAb was added to each well of a 96-well plate; tumor cells were diluted to 2×10e6/ml and seeded in 96-well round plates (100 μl/well); CART cells were diluted to 1×10e6/ml and seeded in 96-well round plates (100 μl/well); the plates were centrifuged at 500 rpm×5 min to attach cells well and cultured at 37° C. for 1 hour; Golgi stop was diluted by 1500× with medium and added to each well (20 μl/well); cells were cultured at 37° C. for another 2.5 hours, stained with anti-CD3-APC and anti-CD8-FITC antibodies at 37° C. for 30 min, washed and analyze by flow cytometry.

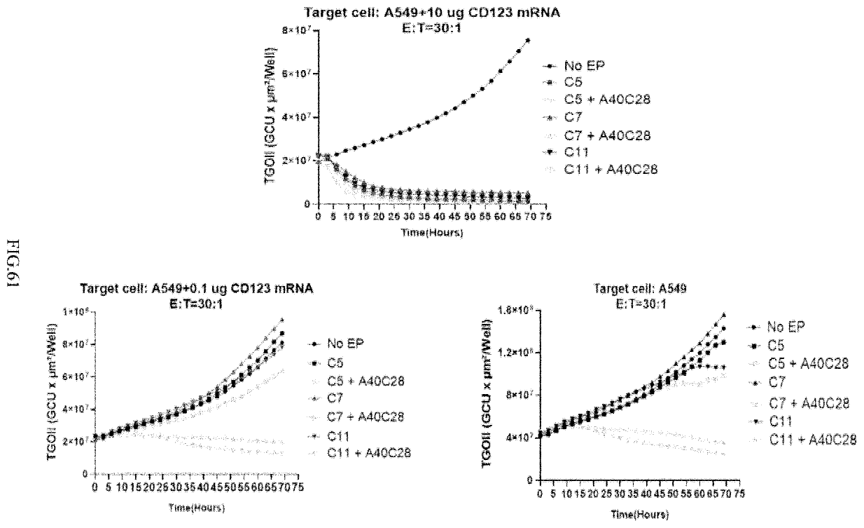

In our studies, activation of CARTs (expressing anti-CD123-C5, anti-CD123-C7, anti-CD123-C11) by CD123-expressing tumor cells was measured by CD107a staining. Tested cells including A549 electroporated with 10 μg, 0.1 μg and 0 μg CD123 mRNA, SK-OV3, PC-3, cord blood derived CD34+ hematopoietic stem cells (CD34+ cord), bone marrow derived hematopoietic stem cells (CD34+M), Molm-14, Nalm6, Jeko-1 tumor cell lines and fresh isolated patient AML tumor cells (CD123+). As shown in FIG. 59, CART cells expressing anti-CD123-C5, -C7 and -C11 were specifically activated by tumor cells having relatively high CD123 expression, especially the CD123+ AML tumor cells, but not tumor cell lines with low CD123 expression. These results indicated that CD123 expression by tumor cells could activate CD123 CARTs.

### 5.3.2: Tumor Killing of LACO-Expressing CD123 CART Cells

The cytolytic activities of the provided CD123 CARTs cells were measured in the tumor killing assay. LACOSTIM A40C28 was used in this study. Various mRNA-based anti-CD123 CART cells, including mock T cells (NO EP), T cells expressing C5 CAR (SEQ ID NO:494), C5 CAR with A40C28 (SEQ ID NO:600), C7 CAR (SEQ ID NO:495), C7 CAR with A40C28, C11 CAR (SEQ ID NO:496), and C11 CAR with A40C28, were co-cultured with tumor cells Molm-14, Nalm6, Jeko-1, at E/T ratio=10:1. As shown in FIG. 60, the co-expression of LACO (A40C28) improved the killing efficiency of provided CART cells against all tumor cells.

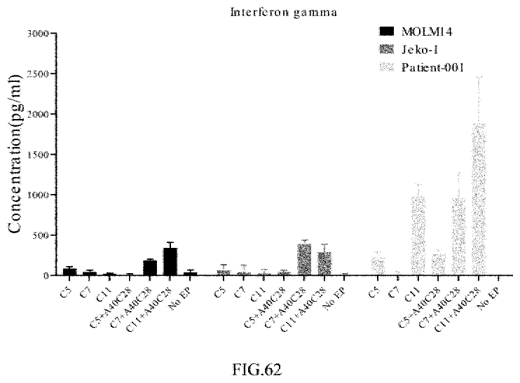

The cytolytic activity of the provided CD123 CARTs cells was further examined in A549 cells electroporated with 0, 0.1 μg or 10 μg CD123 mRNA. As shown in FIG. 62, the ectopic expression levels of CD123 in A549 cells correlated with cytolytic activities of the CD123 CARTs against such tumors. Again, the co-expression of LACO consistently enhanced the anti-tumor effects of the CART cells (FIG. 61).

IFN-γ release was detected by ELISA in the CART killing assays. As shown in FIG. 62, CD123 expressing cancer cells, such as MOLM14 cells, and especially AML cells (patient-001), promoted the release of IFN-γ by the CART cells; and the co-expression of LACO (A40C28) further enhanced such release.

### Example 6: CD40 LACO

### 6.1 Preparation of Anti-Human CD40 Monoclonal Antibodies

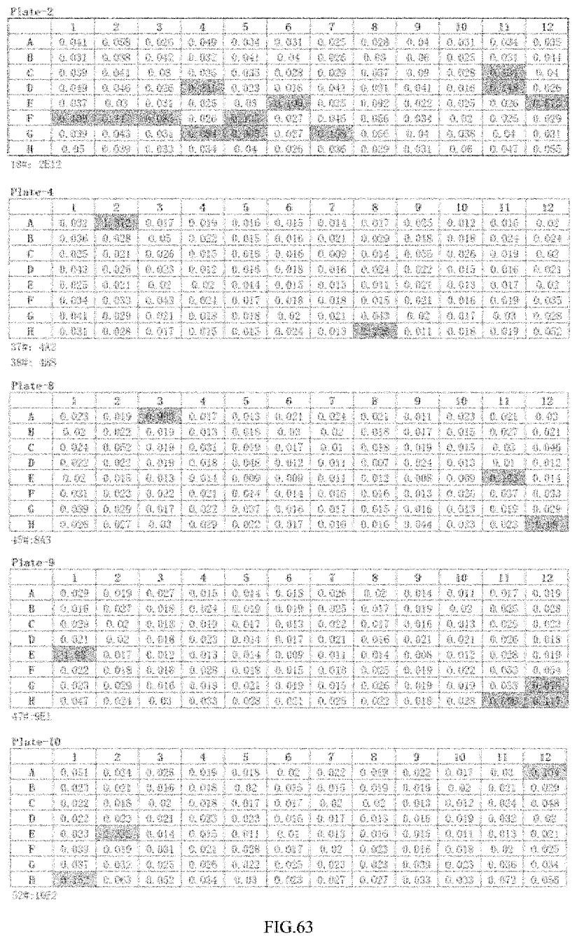

Anti-CD40 antibodies were prepared using fully human antibody phage display library following the steps below: (1) Expression and purification of phage display library; (2) Selection of CD40-specific scFv-phages; (3) mpELISA screening: after three round selection, positive colonies were selected for monoclonal phage ELISA (mpELISA) screening. Phage supernatant was generated from individual bacterial clones and tested for the binding to CD40-6His protein. The supernatant was incubated with pre-blocked Maxisorp plate coated with 2 g/ml CD40-6His protein. After three washes, 100 μl/well of HRP-conjugated anti-M13 antibody diluted 1:5000 in blocking buffer (5% milk+1% BSA in 1×PBS) was added and incubate for 60 min at RT. After washing plate 5 times with PBST, 100 μl/well TMB substrate solution was added and incubated for 10-30 min until blue color had appeared. Reaction was stopped by adding 50 μl/well of stop solution (2N H2SO4). Absorbance was read at 450 nm in a microplate reader. FIG. 63 shows five representative 96-well plates of anti-human CD40-Fc monoclonal phage ELISA.

Cloning and sequence analysis: A total of 56 positive clones were selected according to the ELISA results, and used as templates for PCR cloning of the scFv sequence. The CDR regions of scFv were analyzed through abysis website (http://abysis.org/) according to Kabat numbering scheme, and are provided in list of SEQ ID NOs: 828-863.

6.2 Preparation of CD40 scFv-CD28 Fusion (LACO)

The CD40 scFv-CD28 fusion was synthesized by Sangon Biotech (Shanghai, China). Next, the pUC57-CAR plasmid was linearized by digestion with Spe1 enzyme. The completeness of the digestion was checked by running agarose DNA gel. The linearized vector was purified using PCR Cleanup kit (#28106, QIAGEN) and eluted with EB from the kit water. The concentration of DNA was measured by nanodrop. Then, in vitro transcription (IVT) was performed following the protocol of manufacturer (Thermofisher, Cat No: AM13455). For one reaction, 1 μg template DNA, NTP/ARCA buffer, T7 buffer, GTP, T7 enzyme and Rnase free H2O were added to 0.2 ml PCR tube and incubated at 37° C. for 3 hours. 3 hours later, 2 μl Dnase was added per reaction, and incubated at 37° C. for 15 min. The tailing procedure was performed according to the manufacturer's suggestion. The IVT mRNA was purified using the Rneasy Mini kit (#74106, QIAGEN), and eluted with Rnase-free water. The concentration of RNA was measured by nanodrop. RNA integrity and size were examined by agarose gel electrophoresis.

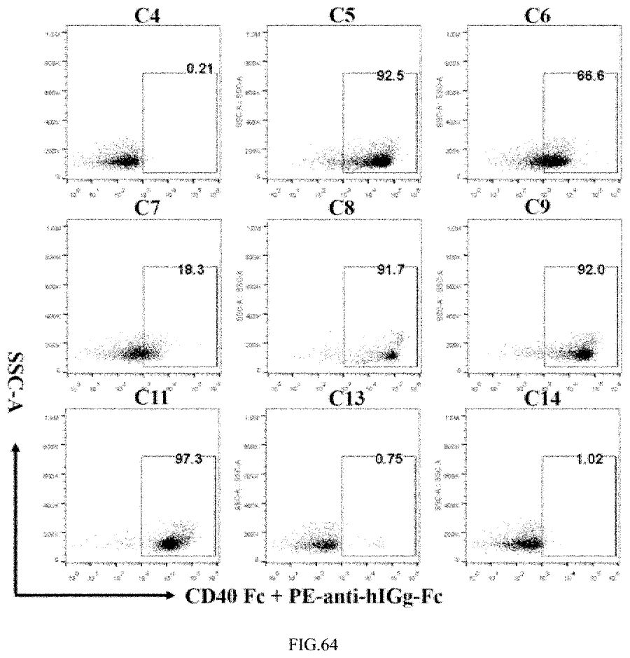

Binding of the anti-CD40 scFv expressed on CARTs cells to CD40-Fc protein was measured by FACS staining. As shown in FIG. 64, C5, C7, C8, and C9 showed strong binding to CD40-Fc recombinant protein.

### 6.3 Tumor Cell Lines and Primary Human Lymphocytes

A549-ESO-CBG cell line was generated by using lentiviral transduction of A549 cells with Click beetle green (CBG) and EGFP, followed by lentiviral transduction of HLA-A2. Primary lymphocytes from normal donors were stimulated with anti-CD3/CD28 Dynabeads (Life Technologies) and cultured in R10 medium (RPMI-1640 supplemented with 10% FCS; Invitrogen). T cells were cryopreserved at day 10 after stimulation in a solution of 90% FCS and 10% DMSO at 1e8 cells/vial.

### 6.4 Preparation and Characterization of LACO-Expressing CART Cells

CART cells expressing LACO provided herein were prepared by electroporation with the following procedures: T cells were collected and washed with Opti-MEM medium for 3 times. The cell pellets were resuspended with Opti-MEM medium, and the cell concentration was adjusted to 5×10⁷/ml. Certain amount of RNA was aliquoted to 1.5 ml EP tube, added with 100 μl T cells (>5×10⁶ cells), and mixed gently to avoid bubbles. Electroporation was performed using BTX machine at the following parameters for T cells: 500 voltage, 0.7 ms, for one pulse. The cells were then transferred to pre-warmed culture medium and cultured at 37° C.

TABLE 6
RNA Used in this study.

| ID | RNA (10 μg each) |
|---|---|
| C4 | 40-18.28 + 4D5.BBZ |
| C5 | 40-37.28 + 4D5.BBZ |
| C6 | 40-38.28 + 4D5.BBZ |
| C7 | 40-45.28 + 4D5.BBZ |
| C8 | 40-47.28 + 4D5.BBZ |
| C9 | 40-52.28 + 4D5.BBZ |
| C11 | A40C28 + 4D5.BBZ |
| C13 | 4D5.BBZ |
| C14 | NO EP |

4D5: anti-Her2 scFv; 4D5.BBZ: anti-Her2 CAR having 4D5, 4-1BB costimulatory domain and CD3ζ signaling domain; 40-18.28: the LACOSTIM having the anti-CD40 scFv 40-18 fused with the intracellular domain of CD28 (same for the other listed LACOSTIMs 40-37.28, 40-37.28, 40-37.28, 40-37.28, 40-37.28, 40-37.28); A40C28: the LACOSTIM having anti-CD40 scFv A40C fused with the intracellular domain of CD28; NO EP: T cells without CAR.

The cytotoxicity of the LACO-expressing CART cells against tumor cells was measured in in vitro cytotoxicity assay. A549-ESO-CGB cells were adjusted to 30,000/ml and seeded to flat-bottomed 96-well plate at 3000 cells/100 μl/well. CART cells were diluted to appropriate concentration, seeded at 100 μl/well with tumor cells at different E/T ratios, such as 10:1, 3:1, 1:1, or 0.3:1. Care was taken to avoid bubbles. The co-culture plates were placed into IncuCyte S3 machine, and scanning parameters were set. After 3 days of scanning, the Total Green Object Integrated Intensity (GCU×μm² μl/well) was analyzed to calculate the killing efficiency.

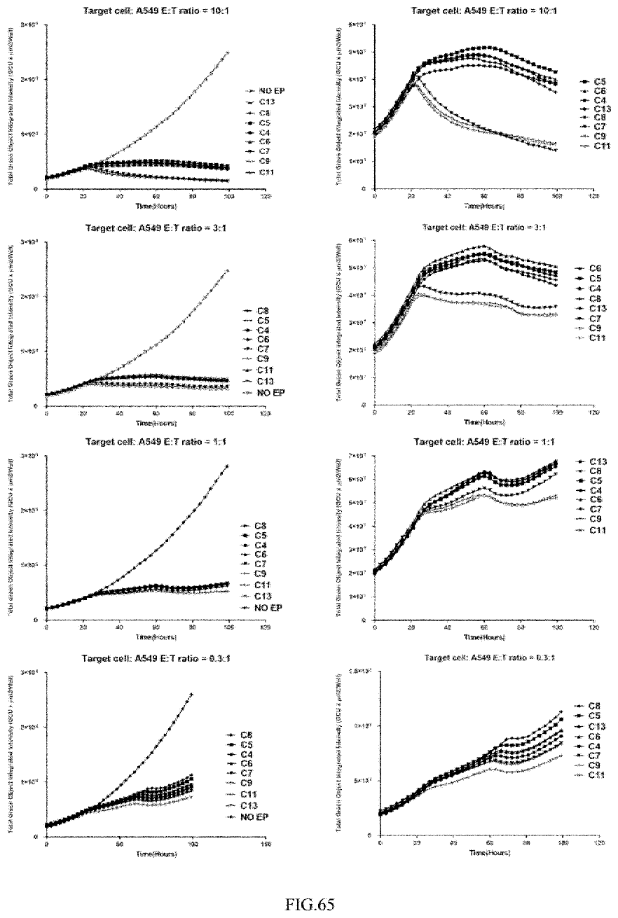

As shown in FIG. 65, T cells C7, C9 or C11 showed significantly enhanced killing effect against tumor cells compared to T cells expressing Her2 CAR alone, confirming that co-expression of respective LACOSTIMs enhanced tumor killing effect of the CART cells.

### 6.5 Specific Activation of LACO-Expressing CART Cells by Cancer Cells

CD107a is an early phase-activating marker for T cells. Activation of CART cells by tumor cells was measured by CD107a staining with the following procedures: 20 μl PE-CD107a mAb was added to each well of a 96-well plate; tumor cells were diluted to 2×10⁶/ml and seeded on 96-well round plates (100 μl/well); CART cells were diluted to 1×10⁶/ml and seeded in 96-well round plates (100 μl/well); the plates were centrifuged at 500 rpm×5 min to attach cells and cultured at 37° C. for 1 hour; Golgi stop was diluted by 1500× with medium and added to each well (20 μl/well); cells were cultured at 37° C. for another 2.5 hours, stained with anti-CD3-APC and anti-CD8-FITC antibodies at 37° C. for 30 min, washed and analyze by flow cytometry.

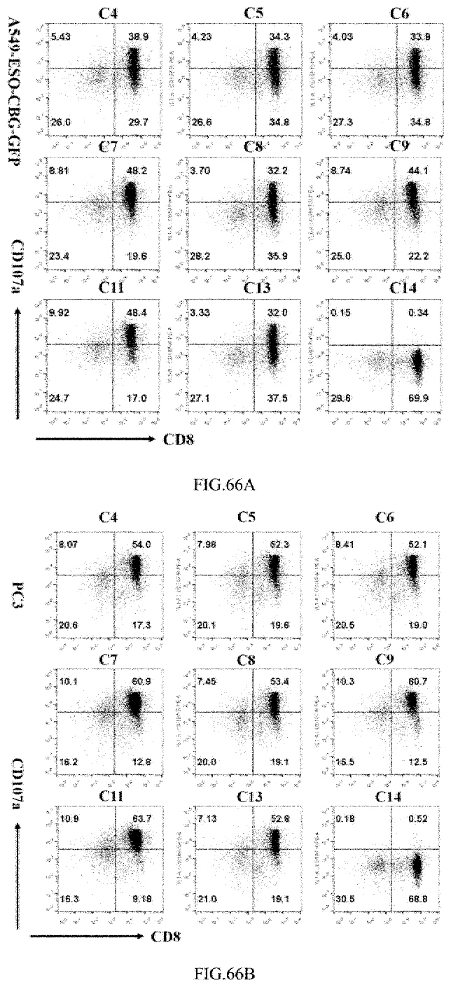

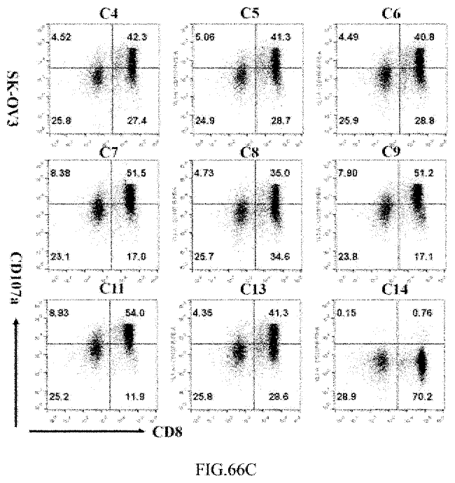

FIGS. 66A-66C show CD107a staining of CAR-T cells in the coculture and killing assay with A549 (FIG. 66A), PC-3 (FIG. 66B), and SK-OV3 (FIG. 66C). As shown, higher percentages of T cells C7, C9 and C11 were activated by the coculture with tumor cells, confirming that co-expression of the respectively LACOSTIMs enhanced tumor-induced activation of CAR T cells.

The sequences of the screened anti-CD40 antibodies (40-18, 40-37, 40-38, 40-45, 40-47 and 40-52) and the corresponding LACOSTIMs in Example 4 shown in Table 7.

TABLE 7
(Kabat)

| SEQ ID | Amino acid |
|---|---|
| NO | sequence |
| 828 | 40-18 VL |
| | CDR1 |
| 829 | 40-37 VL |
| | CDR1 |
| 830 | 40-38 VL |
| | CDR1 |
| 831 | 40-45 VL |
| | CDR1 |
| 832 | 40-47 VL |
| | CDR1 |
| 833 | 40-52 VL |
| | CDR1 |
| 834 | 40-18 VL |
| | CDR2 |
| 835 | 40-37 VL |
| | CDR2 |
| 836 | 40-38 VL |
| | CDR2 |
| 837 | 40-45 VL |
| | CDR2 |
| 838 | 40-47 VL |
| | CDR2 |
| 839 | 40-52 VL |
| | CDR2 |
| 840 | 40-18 VL |
| | CDR3 |
| 841 | 40-37 VL |
| | CDR3 |
| 842 | 40-38 VL |
| | CDR3 |
| 843 | 40-45 VL |
| | CDR3 |
| 844 | 40-47 VL |
| | CDR3 |
| 845 | 40-52 VL |
| | CDR3 |
| 846 | 40-18 VH |
| | CDR1 |
| 847 | 40-37 VH |
| | CDR1 |
| 848 | 40-38 VH |
| | CDR1 |
| 849 | 40-45 VH |
| | CDR1 |
| 850 | 40-47 VH |
| | CDR1 |
| 851 | 40-52 VH |
| | CDR1 |
| 852 | 40-18 VH |
| | CDR2 |
| 853 | 40-37 VH |
| | CDR2 |
| 854 | 40-38 VH |
| | CDR2 |
| 855 | 40-45 VH |
| | CDR2 |
| 856 | 40-47 VH |
| | CDR2 |
| 857 | 40-52 VH |
| | CDR2 |
| 858 | 40-18 VH |
| | CDR3 |
| 859 | 40-37 VH |
| | CDR3 |
| 860 | 40-38 VH |
| | CDR3 |
| 861 | 40-45 VH |
| | CDR3 |
| 862 | 40-47 VH |
| | CDR3 |
| 863 | 40-52 VH |
| | CDR3 |
| 864 | 40-18 VL |
| 865 | 40-37 VL |
| 866 | 40-38 VL |
| 867 | 40-45 VL |
| 868 | 40-47 VL |
| 869 | 40-52 VL |
| 870 | 40-18 VH |
| 871 | 40-37 VH |
| 872 | 40-38 VH |
| 873 | 40-45 VH |
| 874 | 40-47 VH |
| 875 | 40-52 VH |
| 876 | 40-18 VL NT |
| 877 | 40-37 VL NT |
| 878 | 40-38 VL NT |
| 879 | 40-45 VL NT |
| 880 | 40-47 VL NT |
| 881 | 40-52 VL NT |
| 882 | 40-18 VH NT |
| 883 | 40-37 VH NT |
| 884 | 40-38 VH NT |
| 885 | 40-45 VH NT |
| 886 | 40-47 VH NT |
| 887 | 40-52 VH NT |
| 888 | 40-18 scFv |
| 889 | 40-37 scFv |
| 890 | 40-38 scFv |
| 891 | 40-45 scFv |
| 892 | 40-47 scFv |
| 893 | 40-52 scFv |
| 894 | 40-18.28 |
| 895 | 40-37.28 |
| 896 | 40-38.28 |
| 897 | 40-45.28 |
| 898 | 40-47.28 |
| 899 | 40-52.28 |
| 900 | 40-18.28 NT |
| 901 | 40-37.28 NT |
| 902 | 40-38.28 NT |
| 903 | 40-45.28 NT |
| 904 | 40-47.28 NT |
| 905 | 40-52.28 NT |

1. A circular RNA comprising, in the following order, an internal ribosome entry site (IRES) element, a protein coding sequence and a poly A.

2. The circular RNA of claim 1, wherein the polyA is at least 45 or at least 70 nucleotides in length.

3.-4. (canceled)

5. The circular RNA of claim 1, wherein the protein comprises an antigen, an antibody, a chimeric antigen receptor (CAR) or a T cell receptor (TCR).

6. (canceled)

7. The circular RNA of claim 5, wherein the protein comprises an antibody or a CAR comprising the antibody as a binding domain, wherein the antibody specifically binds to mesothelin, CD123, BCMA, CD19, HER2, IL13Ra2, B7H3 or CD40.

8. The circular RNA of claim 7, wherein the antibody specifically binding to mesothelin includes a light chain variable region comprising LCDR1, LCDR2 and LCDR3 and a heavy chain variable region comprising HCDR1, HCDR2, and HCDR3, wherein LCDR1, LCDR2, LCDR3, HCDR1, HCDR2, and HCDR3 are selected from the following group:
(1) a LCDR1 as set forth in SEQ ID NO: 1, a LCDR2 as set forth in SEQ ID NO: 16, a LCDR3 as set forth in SEQ ID NO: 30, a HCDR1 as set forth in SEQ ID NO: 45, a HCDR2 as set forth in SEQ ID NO: 58 and a HCDR3 as set forth in SEQ ID NO: 71;
(2) a LCDR1 as set forth in SEQ ID NO: 2, a LCDR2 as set forth in SEQ ID NO: 17, a LCDR3 as set forth in SEQ ID NO: 31, a HCDR1 as set forth in SEQ ID NO: 46, a HCDR2 as set forth in SEQ ID NO: 59 and a HCDR3 as set forth in SEQ ID NO: 72;
(3) a LCDR1 as set forth in SEQ ID NO: 3, a LCDR2 as set forth in SEQ ID NO: 18, a LCDR3 as set forth in SEQ ID NO: 32, a HCDR1 as set forth in SEQ ID NO: 47, a HCDR2 as set forth in SEQ ID NO: 60 and a HCDR3 as set forth in SEQ ID NO: 73;
(4) a LCDR1 as set forth in SEQ ID NO: 4, a LCDR2 as set forth in SEQ ID NO: 19, a LCDR3 as set forth in SEQ ID NO: 33, a HCDR1 as set forth in SEQ ID NO: 48, a HCDR2 as set forth in SEQ ID NO: 61 and a HCDR3 as set forth in SEQ ID NO: 74;
(5) a LCDR1 as set forth in SEQ ID NO: 5, a LCDR2 as set forth in SEQ ID NO: 20, a LCDR3 as set forth in SEQ ID NO: 34, a HCDR1 as set forth in SEQ ID NO: 49, a HCDR2 as set forth in SEQ ID NO: 62 and a HCDR3 as set forth in SEQ ID NO: 75;
(6) a LCDR1 as set forth in SEQ ID NO: 6, a LCDR2 as set forth in SEQ ID NO: 21, a LCDR3 as set forth in SEQ ID NO: 35, a HCDR1 as set forth in SEQ ID NO: 50, a HCDR2 as set forth in SEQ ID NO: 63 and a HCDR3 as set forth in SEQ ID NO: 76;
(7) a LCDR1 as set forth in SEQ ID NO: 7, a LCDR2 as set forth in SEQ ID NO: 22, a LCDR3 as set forth in SEQ ID NO: 36, a HCDR1 as set forth in SEQ ID NO: 51, a HCDR2 as set forth in SEQ ID NO: 64 and a HCDR3 as set forth in SEQ ID NO: 77;
(8) a LCDR1 as set forth in SEQ ID NO: 8, a LCDR2 as set forth in SEQ ID NO: 23, a LCDR3 as set forth in SEQ ID NO: 37, a HCDR1 as set forth in SEQ ID NO: 52, a HCDR2 as set forth in SEQ ID NO: 65 and a HCDR3 as set forth in SEQ ID NO: 78;
(9) a LCDR1 as set forth in SEQ ID NO: 9, a LCDR2 as set forth in SEQ ID NO: 24, a LCDR3 as set forth in SEQ ID NO: 38, a HCDR1 as set forth in SEQ ID NO: 53, a HCDR2 as set forth in SEQ ID NO: 66 and a HCDR3 as set forth in SEQ ID NO: 79;
(10) a LCDR1 as set forth in SEQ ID NO: 10, a LCDR2 as set forth in SEQ ID NO: 25, a LCDR3 as set forth in SEQ ID NO: 39, a HCDR1 as set forth in SEQ ID NO: 48, a HCDR2 as set forth in SEQ ID NO: 61 and a HCDR3 as set forth in SEQ ID NO: 80;
(11) a LCDR1 as set forth in SEQ ID NO: 11, a LCDR2 as set forth in SEQ ID NO: 26, a LCDR3 as set forth in SEQ ID NO: 40, a HCDR1 as set forth in SEQ ID NO: 54, a HCDR2 as set forth in SEQ ID NO: 67 and a HCDR3 as set forth in SEQ ID NO: 81;
(12) a LCDR1 as set forth in SEQ ID NO: 12, a LCDR2 as set forth in SEQ ID NO: 27, a LCDR3 as set forth in SEQ ID NO: 41, a HCDR1 as set forth in SEQ ID NO: 53, a HCDR2 as set forth in SEQ ID NO: 66 and a HCDR3 as set forth in SEQ ID NO: 82;
(13) a LCDR1 as set forth in SEQ ID NO: 13, a LCDR2 as set forth in SEQ ID NO: 28, a LCDR3 as set forth in SEQ ID NO: 42, a HCDR1 as set forth in SEQ ID NO: 55, a HCDR2 as set forth in SEQ ID NO: 68 and a HCDR3 as set forth in SEQ ID NO: 83;
(14) a LCDR1 as set forth in SEQ ID NO: 14, a LCDR2 as set forth in SEQ ID NO: 29, a LCDR3 as set forth in SEQ ID NO: 43, a HCDR1 as set forth in SEQ ID NO: 56, a HCDR2 as set forth in SEQ ID NO: 69 and a HCDR3 as set forth in SEQ ID NO: 84; and
(15) a LCDR1 as set forth in SEQ ID NO: 15, a LCDR2 as set forth in SEQ ID NO: 18, a LCDR3 as set forth in SEQ ID NO: 44, a HCDR1 as set forth in SEQ ID NO: 57, a HCDR2 as set forth in SEQ ID NO: 70 and a HCDR3 as set forth in SEQ ID NO: 85.

9. The circular RNA of claim 8, wherein the antibody specifically binding to mesothelin comprises a light chain variable region and a heavy chain variable region selected from the following group:
(1) a light chain variable region as set forth in SEQ ID NO: 86 and a heavy chain variable region as set forth in SEQ ID NO: 101;
(2) a light chain variable region as set forth in SEQ ID NO: 87 and a heavy chain variable region as set forth in SEQ ID NO: 102;
(3) a light chain variable region as set forth in SEQ ID NO: 88 and a heavy chain variable region as set forth in SEQ ID NO: 103;
(4) a light chain variable region as set forth in SEQ ID NO: 89 and a heavy chain variable region as set forth in SEQ ID NO: 104;
(5) a light chain variable region as set forth in SEQ ID NO: 90 and a heavy chain variable region as set forth in SEQ ID NO: 105;
(6) a light chain variable region as set forth in SEQ ID NO: 91 and a heavy chain variable region as set forth in SEQ ID NO: 106;
(7) a light chain variable region as set forth in SEQ ID NO: 92 and a heavy chain variable region as set forth in SEQ ID NO: 107;
(8) a light chain variable region as set forth in SEQ ID NO: 93 and a heavy chain variable region as set forth in SEQ ID NO: 108;
(9) a light chain variable region as set forth in SEQ ID NO: 94 and a heavy chain variable region as set forth in SEQ ID NO: 109;
(10) a light chain variable region as set forth in SEQ ID NO: 95 and a heavy chain variable region as set forth in SEQ ID NO: 100;
(11) a light chain variable region as set forth in SEQ ID NO: 96 and a heavy chain variable region as set forth in SEQ ID NO: 111;
(12) a light chain variable region as set forth in SEQ ID NO: 97 and a heavy chain variable region as set forth in SEQ ID NO: 112;
(13) a light chain variable region as set forth in SEQ ID NO: 98 and a heavy chain variable region as set forth in SEQ ID NO: 113;
(14) a light chain variable region as set forth in SEQ ID NO: 99 and a heavy chain variable region as set forth in SEQ ID NO: 114; and
(15) a light chain variable region as set forth in SEQ ID NO: 100 and a heavy chain variable region as set forth in SEQ ID NO: 115.

10.-13. (canceled)

14. The circular RNA of claim 7, wherein the antibody specifically binding to BCMA includes a light chain variable region comprising LCDR1, LCDR2 and LCDR3 and a heavy chain variable region comprising HCDR1, HCDR2, and HCDR3, wherein LCDR1, LCDR2, LCDR3, HCDR1, HCDR2, and HCDR3 are selected from the following group:
(1) a LCDR1 as set forth in SEQ ID NO: 146, a LCDR2 as set forth in SEQ ID NO: 156, a LCDR3 as set forth in SEQ ID NO: 167, a HCDR1 as set forth in SEQ ID NO: 178, a HCDR2 as set forth in SEQ ID NO: 189 and a HCDR3 as set forth in SEQ ID NO: 201;
(2) a LCDR1 as set forth in SEQ ID NO: 147, a LCDR2 as set forth in SEQ ID NO: 157, a LCDR3 as set forth in SEQ ID NO: 168, a HCDR1 as set forth in SEQ ID NO: 179, a HCDR2 as set forth in SEQ ID NO: 190 and a HCDR3 as set forth in SEQ ID NO: 202;
(3) a LCDR1 as set forth in SEQ ID NO: 148, a LCDR2 as set forth in SEQ ID NO: 158, a LCDR3 as set forth in SEQ ID NO: 169, a HCDR1 as set forth in SEQ ID NO: 180, a HCDR2 as set forth in SEQ ID NO: 191 and a HCDR3 as set forth in SEQ ID NO: 203;
(4) a LCDR1 as set forth in SEQ ID NO: 149, a LCDR2 as set forth in SEQ ID NO: 159, a LCDR3 as set forth in SEQ ID NO: 169, a HCDR1 as set forth in SEQ ID NO: 181, a HCDR2 as set forth in SEQ ID NO: 192 and a HCDR3 as set forth in SEQ ID NO: 204;
(5) a LCDR1 as set forth in SEQ ID NO: 150, a LCDR2 as set forth in SEQ ID NO: 160, a LCDR3 as set forth in SEQ ID NO: 170, a HCDR1 as set forth in SEQ ID NO: 182, a HCDR2 as set forth in SEQ ID NO: 193 and a HCDR3 as set forth in SEQ ID NO: 205;
(6) a LCDR1 as set forth in SEQ ID NO: 151, a LCDR2 as set forth in SEQ ID NO: 161, a LCDR3 as set forth in SEQ ID NO: 171, a HCDR1 as set forth in SEQ ID NO: 183, a HCDR2 as set forth in SEQ ID NO: 194 and a HCDR3 as set forth in SEQ ID NO: 206;
(7) a LCDR1 as set forth in SEQ ID NO: 152, a LCDR2 as set forth in SEQ ID NO: 162, a LCDR3 as set forth in SEQ ID NO: 172, a HCDR1 as set forth in SEQ ID NO: 180, a HCDR2 as set forth in SEQ ID NO: 195 and a HCDR3 as set forth in SEQ ID NO: 207;
(8) a LCDR1 as set forth in SEQ ID NO: 153, a LCDR2 as set forth in SEQ ID NO: 163, a LCDR3 as set forth in SEQ ID NO: 173, a HCDR1 as set forth in SEQ ID NO: 184, a HCDR2 as set forth in SEQ ID NO: 196 and a HCDR3 as set forth in SEQ ID NO: 208;
(9) a LCDR1 as set forth in SEQ ID NO: 147, a LCDR2 as set forth in SEQ ID NO: 164, a LCDR3 as set forth in SEQ ID NO: 174, a HCDR1 as set forth in SEQ ID NO: 185, a HCDR2 as set forth in SEQ ID NO: 197 and a HCDR3 as set forth in SEQ ID NO: 209;
(10) a LCDR1 as set forth in SEQ ID NO: 147, a LCDR2 as set forth in SEQ ID NO: 165, a LCDR3 as set forth in SEQ ID NO: 175, a HCDR1 as set forth in SEQ ID NO: 186, a HCDR2 as set forth in SEQ ID NO: 198 and a HCDR3 as set forth in SEQ ID NO: 210;
(11) a LCDR1 as set forth in SEQ ID NO: 154, a LCDR2 as set forth in SEQ ID NO: 166, a LCDR3 as set forth in SEQ ID NO: 176, a HCDR1 as set forth in SEQ ID NO: 187, a HCDR2 as set forth in SEQ ID NO: 199 and a HCDR3 as set forth in SEQ ID NO: 211; and
(12) a LCDR1 as set forth in SEQ ID NO: 155, a LCDR2 as set forth in SEQ ID NO: 159, a LCDR3 as set forth in SEQ ID NO: 177, a HCDR1 as set forth in SEQ ID NO: 188, a HCDR2 as set forth in SEQ ID NO: 200 and a HCDR3 as set forth in SEQ ID NO: 212.

15. The circular RNA of claim 14, wherein the antibody specifically binding to BCMA comprises a light chain variable region and a heavy chain variable region selected from the following group:
(1) a light chain variable region as set forth in SEQ ID NO: 213 and a heavy chain variable region as set forth in SEQ ID NO: 225;
(2) a light chain variable region as set forth in SEQ ID NO: 214 and a heavy chain variable region as set forth in SEQ ID NO: 226;
(3) a light chain variable region as set forth in SEQ ID NO: 215 and a heavy chain variable region as set forth in SEQ ID NO: 227;
(4) a light chain variable region as set forth in SEQ ID NO: 216 and a heavy chain variable region as set forth in SEQ ID NO: 228;
(5) a light chain variable region as set forth in SEQ ID NO: 217 and a heavy chain variable region as set forth in SEQ ID NO: 229;
(6) a light chain variable region as set forth in SEQ ID NO: 218 and a heavy chain variable region as set forth in SEQ ID NO: 230;
(7) a light chain variable region as set forth in SEQ ID NO: 219 and a heavy chain variable region as set forth in SEQ ID NO: 231;
(8) a light chain variable region as set forth in SEQ ID NO: 220 and a heavy chain variable region as set forth in SEQ ID NO: 232;
(9) a light chain variable region as set forth in SEQ ID NO: 221 and a heavy chain variable region as set forth in SEQ ID NO: 233;
(10) a light chain variable region as set forth in SEQ ID NO: 222 and a heavy chain variable region as set forth in SEQ ID NO: 234;
(11) a light chain variable region as set forth in SEQ ID NO: 223 and a heavy chain variable region as set forth in SEQ ID NO: 235; and
(12) a light chain variable region as set forth in SEQ ID NO: 224 and a heavy chain variable region as set forth in SEQ ID NO: 236.

16.-31. (canceled)

32. The circular RNA of claim 7, wherein the CAR is a CAR targeting mesothelin, which comprises an amino acid sequence selected from SEQ ID Nos: 131-145.

33. The circular RNA of claim 1, wherein the protein comprises a fusion protein and the fusion protein comprises a first domain that activates an antigen-presenting cell (APC) and a second domain that activates an immune effector cell, wherein the first domain comprises CD40L or a receptor-binding fragment thereof, an anti-CD40 antibody or an antigen-binding fragment thereof, and the second domain comprises a CD28 cytoplasmic domain or an anti-CD28 antibody or an antigen-binding fragment thereof.

34. (canceled)

35. The circular RNA of claim 33, wherein the first domain is linked to the N-terminus or C-terminus of the second domain.

36. The circular RNA of claim 33, wherein
the first domain and the second domain are linked via a linker.

37. (canceled)

38. The circular RNA of claim 33, wherein the anti-CD40 antibody includes a light chain variable region comprising LCDR1, LCDR2 and LCDR3 and a heavy chain variable region comprising HCDR1, HCDR2, and HCDR3, wherein LCDR1, LCDR2, LCDR3, HCDR1, HCDR2, and HCDR3 are selected from the following group:
(1) a LCDR1 as set forth in SEQ ID NO: 828, a LCDR2 as set forth in SEQ ID NO: 834, a LCDR3 as set forth in SEQ ID NO: 840, a HCDR1 as set forth in SEQ ID NO: 846, a HCDR2 as set forth in SEQ ID NO: 852 and a HCDR3 as set forth in SEQ ID NO: 858;
(2) a LCDR1 as set forth in SEQ ID NO: 829, a LCDR2 as set forth in SEQ ID NO: 835, a LCDR3 as set forth in SEQ ID NO: 841, a HCDR1 as set forth in SEQ ID NO: 847, a HCDR2 as set forth in SEQ ID NO: 853 and a HCDR3 as set forth in SEQ ID NO: 859;
(3) a LCDR1 as set forth in SEQ ID NO: 830, a LCDR2 as set forth in SEQ ID NO: 836, a LCDR3 as set forth in SEQ ID NO: 842, a HCDR1 as set forth in SEQ ID NO: 848, a HCDR2 as set forth in SEQ ID NO: 854 and a HCDR3 as set forth in SEQ ID NO: 860;
(4) a LCDR1 as set forth in SEQ ID NO: 831, a LCDR2 as set forth in SEQ ID NO: 837, a LCDR3 as set forth in SEQ ID NO: 843, a HCDR1 as set forth in SEQ ID NO: 849, a HCDR2 as set forth in SEQ ID NO: 855 and a HCDR3 as set forth in SEQ ID NO: 861;
(5) a LCDR1 as set forth in SEQ ID NO: 832, a LCDR2 as set forth in SEQ ID NO: 838, a LCDR3 as set forth in SEQ ID NO: 844, a HCDR1 as set forth in SEQ ID NO: 850, a HCDR2 as set forth in SEQ ID NO: 856 and a HCDR3 as set forth in SEQ ID NO: 862; and
(6) a LCDR1 as set forth in SEQ ID NO: 833, a LCDR2 as set forth in SEQ ID NO: 839, a LCDR3 as set forth in SEQ ID NO: 845, a HCDR1 as set forth in SEQ ID NO: 851, a HCDR2 as set forth in SEQ ID NO: 857 and a HCDR3 as set forth in SEQ ID NO: 863.

39. The circular RNA of claim 33, wherein the anti-CD40 antibody comprises a light chain variable region and a heavy chain variable region selected from the following group:
(1) a light chain variable region as set forth in SEQ ID NO: 864 and a heavy chain variable region as set forth in SEQ ID NO: 870;
(2) a light chain variable region as set forth in SEQ ID NO: 865 and a heavy chain variable region as set forth in SEQ ID NO: 97-4871;
(3) a light chain variable region as set forth in SEQ ID NO: 866 and a heavy chain variable region as set forth in SEQ ID NO: 872;
(4) a light chain variable region as set forth in SEQ ID NO: 867 and a heavy chain variable region as set forth in SEQ ID NO: 873;
(5) a light chain variable region as set forth in SEQ ID NO: 868 and a heavy chain variable region as set forth in SEQ ID NO: 874; and
(6) a light chain variable region as set forth in SEQ ID NO: 869 and a heavy chain variable region as set forth in SEQ ID NO: 875.

40.-41. (canceled)

42. A precursor RNA for producing the circular RNA of claim 1, the precursor RNA comprising a circularizing element, an internal ribosome entry site (IRES) element, a protein coding sequence and a poly A.

43.-49. (canceled)

50. A method of producing a circular RNA, the method comprising circularizing the precursor RNA of claim 42 to produce the circular RNA, and purifying the circular RNA through oligo dT-based capturing.

51.-54. (canceled)

55. A cell or a cell population comprising the circular RNA of claim 1.

56. A cell or a cell population comprising a first circular RNA and a second circular RNA, wherein the first circular RNA is a circular RNA of claim 5; and the second circular RNA is a circular RNA comprising, in the following order, an internal ribosome entry site (IRES) element, a sequence encoding a fusion protein, and a poly A, wherein the fusion protein comprises a first domain that activates an antigen-presenting cell (APC) and a second domain that activates an immune effector cell, wherein the first domain comprises CD40L or a receptor-binding fragment thereof, an anti-CD40 antibody or an antigen-binding fragment thereof, and the second domain comprises a CD28 cytoplasmic domain or an anti-CD28 antibody or an antigen-binding fragment thereof.

57.-63. (canceled)

64. A method of treating a cancer in a subject in need thereof, comprising administering to the subject a therapeutically effective amount of the cell or cell population of claim 55.

65. (canceled)

66. The method of claim 64, wherein the cancer expresses mesothelin, CD123, BCMA, CD19, HER2, IL13Ra2 or B7H3.

67. The method of claim 64, wherein the cancer is a solid tumor or a hematological cancer.

68. The method of claim 64 wherein the cancer is acute myeloid leukemia (AML), B-acute lymphoid leukemia (B-ALL), T-acute lymphoid leukemia (T-ALL), B cell precursor acute lymphoblastic leukemia (BCP-ALL) or blastic plasmacytoid dendritic cell neoplasm (BPDCN), non-Hodgkin's lymphoma, chronic lymphocytic leukemia, acute lymphocytic leukemia, human B-cell precursor leukemia, multiple myeloma or malignant lymphoma, mesothelioma, pancreatic cancer, ovarian cancer, lung cancer, breast cancer, stomach cancer, cervical cancer, uroepithelial cancer, esophageal cancer, bladder cancer, colorectal cancer, endometrial cancer, kidney cancer, head and neck cancer, sarcoma, glioblastoma, prostate cancer, thyroid cancer or glioma.

69.-75. (canceled)
